(19) United States
(12) Patent Application Publication
(10) Pub. No.: US 2026/0078428 A1
(43) Pub. Date: Mar. 19, 2026

(54) WHOLE BLOOD CRYOPRESERVATION AND PROCESSING METHOD FOR SINGLE-CELL RNA-SEQUENCING

(71) Applicants: THE BROAD INSTITUTE, INC., Cambridge, MA (US); THEGENERAL HOSPITAL CORPORATION, Boston, MA (US)

(72) Inventors: Roby BHATTACHARYYA, Boston, MA (US); Michael FILBIN, Boston, MA (US); Alyssa DUBOIS, Cambridge, MA (US); Peter ANKOMAH, Boston, MA (US)

(73) Assignees: THE BROAD INSTITUTE, INC., Cambridge, MA (US); THE GENERAL HOSPITAL CORPORATION, Boston, MA (US)

(21) Appl. No.: 19/329,771

(22) Filed: Sep. 16, 2025

Related U.S. Application Data
(60) Provisional application No. 63/695,116, filed on Sep. 16, 2024.

(51) Int. Cl. C12Q 1/6806; C12N 5/00; C12Q 1/6869

(52) U.S. Cl. CPC C12Q 1/6806; C12N 5/525; C12Q 1/6869

(57) ABSTRACT

The disclosure relates generally to methods and compositions for cryopreservation and processing of blood for single-cell RNA-sequencing. More particularly, the disclosure relates to methods and compositions for preserving and processing whole blood to enable diagnosing and/or treating sepsis via single-cell RNA sequencing (scRNA-seq). In certain aspects, the methods and compositions disclosed herein may be employed in diagnosis and treatment of subjects having or at risk of having sepsis.

### CROSS REFERENCE TO RELATED APPLICATION

The present application claims priority under 35 U.S.C. § 119(e) to U.S. provisional patent application No. 63/695,116, entitled “WHOLE BLOOD CRYOPRESERVATION AND PROCESSING METHOD FOR SINGLE-CELL RNA-SEQUENCING,” filed Sep. 16, 2024. The entire content of the aforementioned patent application is incorporated herein by this reference.

### STATEMENT AS TO FEDERALLY SPONSORED RESEARCH

This invention was made with government support under GM148826 awarded by the National Institutes of Health. The government has certain rights in the invention.

### FIELD OF THE DISCLOSURE

The disclosure relates generally to methods and compositions for preserving and processing whole blood. More particularly, the disclosure relates to methods and compositions for preserving and processing whole blood to enable diagnosing and/or treating sepsis via single-cell RNA sequencing (scRNA-seq).

### BACKGROUND OF THE DISCLOSURE

Single-cell RNA sequencing (scRNA-seq) is a pivotal methodology with great potential for advancing the understanding of biological systems. It has revealed previously unrecognized cell types and transcriptional substates within complex tissues and demonstrated differences in gene expression that illuminate pathophysiological variation in many aspects of human disease. ScRNA-seq has particular appeal for dissecting the cellular basis for heterogeneity in diseases and opening pathways to precision medicine. Sepsis, for example, is a syndrome with expansive differences in clinical course and outcome between patients that is impacted substantially by heterogeneity in patients' immunological responses. However, there has been limited progress in understanding this variation using existing methods. In particular, transcriptional profiling in patients with sepsis has mostly relied on bulk RNA sequencing (bulk RNA-seq) to generate averaged signatures from all circulating immune cells, obscuring the cellular basis and underlying mechanisms of immune dysfunction.

ScRNA-seq has been used to profile circulating peripheral blood mononuclear cells (PBMCs) in urosepsis, identifying a unique CD14+ monocyte subtype (monocyte substate 1, or MS1), that is expanded in sepsis relative to infection without sepsis. These monocytes have a gene expression profile similar to myeloid-derived immune suppressor cells, which are immune regulatory cells that inhibit T cell activation, proliferation and cytotoxic activity. MS1 cells may therefore play an immunosuppressive role in sepsis and contribute to an important transcriptional subphenotype, or “endotype”, of sepsis. Several other studies have employed scRNA-seq in small sepsis cohorts. However, resolution of sepsis endotypes and the contributory roles of immune cell subtypes requires large cohorts enrolled at multiple, geographically-separated clinical sites. Such large-scale studies are necessary to appropriately represent the heterogeneity of sepsis with its variable pathogen types, anatomic sites of infection, timing of presentation, severity, trajectory, clinical characteristics (e.g., age, sex, race and ethnicity, comorbidities), and complex host immune responses. Leveraging the resolution of scRNA-seq at a scale that allows sufficient sampling of all relevant subphenotypes of sepsis has the potential to enable endotyping assessment with sufficient accuracy to impact sepsis care.

Sepsis is prevalent, costly, and deadly. In the U.S., sepsis accounts for 4% of hospitalizations, 13% of in-hospital healthcare expenditures, and 35% of in-hospital deaths. Case mortality ranges from 15 to 34% depending on the degree of organ dysfunction involved, survivors suffer from impaired quality of life and long term-complications are common.

There exists a lack of precision in sepsis definition, diagnosis and disease characterization. Although early detection and intervention are important for improving outcomes in sepsis, identification is often difficult in the acute setting. Sepsis is defined as a clinical syndrome of “life-threatening organ dysfunction caused by a dysregulated host response to infection”, but neither infection nor organ dysfunction may be apparent upon hospital presentation, as previously shown. Current diagnostics are imprecise, relying either on vital signs-based tools like the quick sequential organ dysfunction assessment (qSOFA) score, or lab values like serum lactate, which are neither sensitive nor specific for bacterial sepsis. As a result, patients classified as “septic” in practice and in clinical studies include those with a broad range of possible infectious etiologies and varying degrees of organ dysfunction, not reflective of underlying disease mechanisms, and include patients not even infected at all. Accordingly, there is an urgent need for a practical means of characterizing the host immune response to infection at sufficient depth to enable development of precise biomarker-based sepsis definitions and associated clinical diagnostics.

### SUMMARY OF THE DISCLOSURE

Disclosed herein, in certain embodiments, are methods for the cryopreservation and processing of whole blood. In some embodiments, the cryopreservation and processing of whole blood is for single-cell RNA sequencing (scRNA-seq).

In one aspect, the disclosure provides for a method of cryopreserving a blood sample, including obtaining a blood sample; mixing the blood sample with dimethyl sulfoxide (DMSO) to create a blood sample-DMSO mixture that does not comprise a serum supplement; and freezing the blood sample-DMSO mixture within four hours of obtaining the blood sample. In certain embodiments, the blood sample-DMSO mixture includes between about 5% and about 15% DMSO v/v. In some embodiments, the blood sample-DMSO mixture includes between about 8% and about 12% DMSO v/v. In some embodiments, the blood sample-DMSO mixture includes about 10% DMSO v/v. In some embodiments, the blood sample-DMSO mixture includes a volume percent (v/v) of DMSO of about 5%, about 6%, about 7%, about 8%, about 9%, about 10%, about 11%, about 12%, about 13%, about 14%, or about 15%. In some embodiments, the serum supplement is fetal bovine serum (FBS), newborn calf serum (NCS), horse serum, human serum, platelet lysate, bovine serum albumin (BSA), serum replacement, tryptose phosphate broth (TPB), insulin-transferrin-selenium (ITS), KnockOut™ Serum Replacement (KSR), CryoStor, or any combination thereof.

In some embodiments, the method does not include a centrifugation step. In some embodiments, freezing includes decreasing the temperature of the blood sample-DMSO mixture by at least about 1 degree per minute. In some embodiments, the method further includes the steps of thawing the sample; fluorescence activated cell sorting the sample to isolate and purify peripheral blood mononuclear cells (PBMCs); optionally using single-cell RNA sequencing to sequence the PBMCs; analyzing the scRNA-seq data, thereby identifying a sepsis-specific disease endotype; and optionally selecting a treatment for sepsis in the subject based on the sepsis-specific disease endotype identified. In some embodiments, the blood sample is from a human subject. In some embodiments, the human subject has, is suspected of having, or is at risk of having, sepsis.

In another aspect, the disclosure provides for a method of cryopreserving a blood sample and isolating peripheral blood mononuclear cells (PBMCs) from the blood sample, including obtaining the blood sample; mixing the blood sample with dimethyl sulfoxide (DMSO) to create a blood sample-DMSO mixture that does not comprise a serum supplement; freezing the blood sample-DMSO mixture within four hours of obtaining the blood sample; thawing the blood sample-DMSO mixture; mixing the thawed blood sample-DMSO mixture with a buffer to create a buffered blood sample-DMSO mixture, wherein the buffer comprises phosphate buffered saline (PBS), ethylenediaminetetraacetic acid (EDTA), and a serum supplement; depleting red blood cells from the buffered blood sample-DMSO mixture using a negative selection; and performing flow cytometry on the depleted and buffered blood sample-DMSO mixture to isolate PBMCs. In some embodiments, the blood sample-DMSO mixture includes between about 5% and about 15% DMSO v/v. In some embodiments, the blood sample-DMSO mixture includes between about 8% and about 12% DMSO v/v. In some embodiments, the blood sample-DMSO mixture includes about 10% DMSO v/v. In some embodiments, the blood sample-DMSO mixture includes a volume percent (v/v) of DMSO of about 5%, about 6%, about 7%, about 8%, about 9%, about 10%, about 11%, about 12%, about 13%, about 14%, or about 15%. In some embodiments, the serum supplement is fetal bovine serum (FBS), newborn calf serum (NCS), horse serum, human serum, platelet lysate, bovine serum albumin (BSA), serum replacement, tryptose phosphate broth (TPB), insulin-transferrin-selenium (ITS), KnockOut™ Serum Replacement (KSR), CryoStor, or any combination thereof. In some embodiments, the method does not comprise a centrifugation step. In some embodiments, the depleting step includes immunomagnetic depletion, optionally wherein the immunomagnetic depletion includes using a red blood cell (RBC) depletion reagent.

In some embodiments, the disclosure provides for methods wherein steps of the method occur at different times and/or different places, or wherein steps may occur at one or more hospital sites. In some embodiments, the EDTA molarity is between about 1 mM and about 5 mM. In some embodiments, the EDTA molarity is between about 1 mM and about 3 mM. In some embodiments, the EDTA molarity is about 2 mM. In some embodiments, the FBS volume percent (v/v) is between about 1% and about 5%. In some embodiments, the FBS volume percent (v/v) is between about 1% and about 3%. In some embodiments, the FBS volume percent (v/v) is about 2.5%. In some embodiments, freezing includes decreasing the temperature of the blood sample-DMSO mixture by at least about 1 degree per minute. In some embodiments, thawing includes incubating the blood sample-DMSO mixture at 37° C. for about 1 minute 15 seconds. In some embodiments, the blood sample is from a human subject. In some embodiments, the human subject has, is suspected of having, or is at risk of having, sepsis.

In another aspect, the disclosure provides for a method of assaying peripheral blood mononuclear cells (PBMCs) from a blood sample, the method including obtaining the blood sample from at least one subject; mixing the blood sample with dimethyl sulfoxide (DMSO) to create a blood sample-DMSO mixture that does not comprise a serum supplement; freezing the blood sample-DMSO mixture within four hours of obtaining the blood sample; thawing the blood sample-DMSO mixture; mixing the thawed blood sample-DMSO mixture with a buffer to create a buffered blood sample-DMSO mixture, wherein the buffer comprises phosphate buffered saline (PBS), ethylenediaminetetraacetic acid (EDTA), and a serum supplement; depleting red blood cells from the buffered blood sample-DMSO mixture using a negative selection; performing flow cytometry on the depleted and buffered blood sample-DMSO mixture to isolate PBMCs; and assaying the isolated PBMCs using single-cell RNA sequencing (scRNA-seq). In some embodiments, the blood sample-DMSO mixture includes between about 5% and about 15% DMSO v/v.

In some embodiments, the blood sample-DMSO mixture includes between about 8% and about 12% DMSO v/v. In some embodiments, the blood sample-DMSO mixture includes about 10% DMSO v/v. In some embodiments, the blood sample-DMSO mixture includes a volume percent (v/v) of DMSO of about 5%, about 6%, about 7%, about 8%, about 9%, about 10%, about 11%, about 12%, about 13%, about 14%, or about 15%. In some embodiments, the serum supplement is fetal bovine serum (FBS), newborn calf serum (NCS), horse serum, human serum, platelet lysate, bovine serum albumin (BSA), serum replacement, tryptose phosphate broth (TPB), insulin-transferrin-selenium (ITS), KnockOut™ Serum Replacement (KSR), CryoStor, or any combination thereof. In some embodiments, the method does not include a centrifugation step. In some embodiments, the depleting step includes immunomagnetic depletion, optionally the immunomagnetic depletion includes using a red blood cell (RBC) depletion reagent. In some embodiments, certain of the steps of the method may occur at different times and/or places then other steps, or certain steps may occur at one or more hospital sites. In some embodiments, the EDTA molarity is between about 1 mM and about 5 mM. In some embodiments, the EDTA molarity is between about 1 mM and about 3 mM. In some aspects, the EDTA molarity is about 2 mM. In some embodiments, the FBS volume percent (v/v) is between about 1% and about 5%. In some embodiments, the FBS volume percent (v/v) is between about 1% and about 3%. In some aspects, the FBS volume percent (v/v) is about 2.5%. In some embodiments, freezing includes decreasing the temperature of the blood sample-DMSO mixture by at least about 1 degree per minute. In some embodiments, thawing includes incubating the blood sample-DMSO mixture at 37° C. for between about 1 and 2 minutes. In some embodiments, thawing includes incubating the blood sample-DMSO mixture at 37° C. for about 1 minute 15 seconds. In some embodiments, the scRNA-seq is droplet based scRNA-seq. In some embodiments, the scRNA-seq is on more than one blood sample. In some embodiments of the disclosure, the more than one blood sample is from at least two subjects. In some embodiments, the more than one blood sample is from the same subject. In some embodiments, the scRNA-seq generates an RNA library. In some embodiments, the blood sample is from a human subject. In some embodiments, the human subject has, or is suspected of having, sepsis.

In another aspect, the disclosure provides a method of selecting a treatment for sepsis in a subject in need thereof, the method including identifying a sepsis-specific disease endotype in the subject including incubating the blood sample from the subject with an aprotic solvent, to create a blood sample-aprotic solvent mixture that does not comprise serum; freezing the blood sample-aprotic solvent mixture within four hours of obtaining the blood sample; thawing the blood sample-aprotic solvent mixture; mixing the thawed blood sample-aprotic solvent mixture with a buffer to create a buffered blood sample-aprotic solvent mixture, wherein the buffer comprises phosphate buffered saline (PBS), ethylenediaminetetraacetic acid (EDTA), and a serum supplement; depleting red blood cells from the buffered blood sample-aprotic solvent mixture using a negative selection; performing flow cytometry on the depleted and buffered blood sample-aprotic solvent mixture to isolate PBMCs; assaying the isolated PBMCs using single-cell RNA sequencing; analyzing the scRNA-seq data, thereby identifying a sepsis-specific disease endotype; and selecting a treatment for sepsis in the subject based on the sepsis-specific disease endotype identified. In some embodiments, the blood sample-aprotic solvent mixture includes between about 5% and about 15% of the aprotic solvent v/v. In some embodiments, the blood sample-aprotic solvent mixture includes between about 8% and about 12% of the aprotic solvent v/v. In some embodiments, the blood sample-aprotic solvent mixture includes about 10% of the aprotic solvent v/v. In some embodiments, the blood sample-aprotic solvent mixture includes a volume percent (v/v) of aprotic solvent of about 5%, about 6%, about 7%, about 8%, about 9%, about 10%, about 11%, about 12%, about 13%, about 14%, or about 15%. In some embodiments, the serum supplement is fetal bovine serum (FBS), newborn calf serum (NCS), horse serum, human serum, platelet lysate, bovine serum albumin (BSA), serum replacement, tryptose phosphate broth (TPB), insulin-transferrin-selenium (ITS), KnockOut™ Serum Replacement (KSR), CryoStor, or any combination thereof. In some aspects, the method does not include a centrifugation step. In some embodiments, the depleting step includes immunomagnetic depletion, optionally the immunomagnetic depletion includes using a red blood cell (RBC) depletion reagent. In some aspects, method steps of the disclosure may occur at different times and/or different places. In some embodiments, method steps of the disclosure may occur at one or more hospital sites. In some embodiments, the EDTA molarity is between about 1 mM and about 5 mM. In some embodiments, the EDTA molarity is between about 1 mM and about 3 mM. In some embodiments, the EDTA molarity is about 2 mM. In some embodiments, the FBS volume percent (v/v) is between about 1% and about 5%. In some embodiments, the FBS volume percent (v/v) is between about 1% and about 3%. In some embodiments, the FBS volume percent (v/v) is about 2.5%. In some embodiments, freezing includes decreasing the temperature of the blood sample-aprotic solvent mixture by at least about 1 degree per minute. In some embodiments, thawing includes incubating the blood sample-aprotic solvent mixture at 37° C. for between about 1 and about 2 minutes. In some embodiments, thawing includes incubating the blood sample-aprotic solvent mixture at 37° C. for about 1 minute 15 seconds. In some embodiments, the scRNA-seq is droplet based scRNA-seq. In some embodiments, the scRNA-seq is on more than one blood sample. In some embodiments, the more than one blood sample is from at least two subjects. In some embodiments, the more than one blood sample is from the same subject. In some embodiments, the scRNA-seq generates an RNA library. In some embodiments, the blood sample is from a human subject. In some embodiments, the human subject has, or is suspected of having, sepsis. In some embodiments, the sepsis-specific disease endotype is selected from the group consisting of Molecular Diagnosis and Risk Stratification of Sepsis (MARS) endotypes 1-4; or Sepsis Response Signature (SRS) endotypes 1-2; or among the set of Neutrophilic-Suppressive (NPS), Inflammatory (INF), Innate Host Defence (IHD), Interferon (IFN), and Adaptive (ADA) endotypes, where the MARS endotype includes at least the set of MARS 1, MARS 2, MARS 3, MARS 4, and the SRS endotype includes at least the set of SRS 1 and SRS 2. (Scicluna, Brendon P., et al. “Classification of patients with sepsis according to blood genomic endotype: a prospective cohort study.” The Lancet Respiratory Medicine 5.10 (2017): 816-826; Stanski, Natalja L., and Hector R. Wong. “Prognostic and predictive enrichment in sepsis.” Nature Reviews Nephrology 16.1 (2020): 20-31; Baghela, Arjun, et al. “Predicting sepsis severity at first clinical presentation: The role of endotypes and mechanistic signatures.” EBioMedicine 75 (2022); Davenport, Emma E., et al. “Genomic landscape of the individual host response and outcomes in sepsis: a prospective cohort study.” The Lancet Respiratory Medicine 4.4 (2016): 259-271) In some embodiments of the disclosure, the sepsis disease endotype is associated with neutrophil activation and immune suppression; associated with an increased pro-inflammatory response; associated with an increased NF-κB expression; associated with interleukin signaling; associated with increased IFN-α,β,γ; or associated with a variety of pathways including increased adaptive immunity. In some embodiments, selecting the treatment based on the sepsis-specific disease endotype includes selecting an antibiotic and/or source control. In some embodiments, the selection of treatment is based on identification of the sepsis-specific disease endotype, thereby avoiding unnecessary or potentially harmful treatment protocols. In some embodiments, treatment is administered parenterally, intravenously, orally, topically, subcutaneously, peritoneally, intra-arterially, through inhalation, vaginally, rectally, nasally, into the cerebrospinal fluid, or into a body compartment. In some embodiments, the aprotic solvent is dimethyl sulfoxide (DMSO). In some embodiments, the blood sample-aprotic solvent mixture comprises a volume percent (v/v) of DMSO of between about 5% and about 15%. In some embodiments, the blood sample-aprotic solvent mixture comprises a volume percent (v/v) of DMSO of between about 8% and about 12%. In some embodiments, the blood sample-aprotic solvent mixture comprises a volume percent (v/v) of DMSO of about 10%. In some embodiments, the blood sample-aprotic solvent mixture comprises a volume percent (v/v) of DMSO of about 5%, about 6%, about 7%, about 8%, about 9%, about 10%, about 11%, about 12%, about 13%, about 14%, or about 15%.

In an aspect, the disclosure provides kits for cryopreserving and processing whole blood for single-cell RNA sequencing. Such kits may comprise dimethyl sulfoxide (DMSO), a buffer comprising phosphate buffered saline (PBS), ethylenediaminetetraacetic acid (EDTA), and a serum supplement, a red blood cell depletion reagent, and instructions for use. In some embodiments, the serum supplement may be fetal bovine serum (FBS), newborn calf serum (NCS), horse serum, human serum, platelet lysate, bovine serum albumin (BSA), serum replacement, tryptose phosphate broth (TPB), insulin-transferrin-selenium (ITS), KnockOut™ Serum Replacement (KSR), or CryoStor. In some embodiments, the red blood cell depletion reagent may comprise immunomagnetic beads.

In some embodiments, the EDTA may be present at a concentration of between about 1 mM and about 5 mM in the buffer. In some embodiments, the serum supplement may be present at a volume percent (v/v) of between about 1% and about 5% in the buffer. The kits may further comprise one or more cryovials for storing the blood sample-DMSO mixture. In some embodiments, the kits may additionally include flow cytometry reagents for isolating peripheral blood mononuclear cells (PBMCs). Such flow cytometry reagents may comprise fluorescently labeled antibodies against CD45, CD235a, and CD15. In some embodiments, the kits may further comprise single-cell RNA sequencing reagents.

In some embodiments, the disclosure provides kits for diagnosing sepsis. Such diagnostic kits may comprise dimethyl sulfoxide (DMSO), a buffer comprising phosphate buffered saline (PBS), ethylenediaminetetraacetic acid (EDTA), and a serum supplement, a red blood cell depletion reagent, single-cell RNA sequencing reagents, and instructions for identifying a sepsis-specific disease endotype. The instructions may comprise guidance for identifying a sepsis-specific disease endotype selected from the group consisting of: Molecular Diagnosis and Risk Stratification of Sepsis (MARS) 1, MARS 2, MARS 3, MARS 4, Sepsis Response Signature (SRS) SRS 1, SRS 2, Neutrophilic-Suppressive (NPS), Inflammatory (INF), Innate Host Defence (IHD), Interferon (IFN), and Adaptive (ADA). In some embodiments, the kits may further comprise treatment selection guidance based on identified sepsis-specific disease endotypes.

In some embodiments, the disclosure provides compositions comprising dimethyl sulfoxide (DMSO) and whole blood, wherein the composition does not comprise a serum supplement, and wherein the DMSO is present at a volume percent (v/v) of between about 5% and about 15%. In certain embodiments, the DMSO may be present at a volume percent (v/v) of about 10%. In some embodiments, the whole blood may be from a human subject having, suspected of having, or at risk of having sepsis.

### Definitions

The use of the terms “a” and “an” and “the” and similar referents in the context of describing the disclosure (especially in the context of the following claims) are to be construed to cover both the singular and the plural, unless otherwise indicated herein or clearly contradicted by context.

Unless specifically stated or obvious from context, as used herein, the term “about” is understood as within a range of normal tolerance in the art, for example within 2 standard deviations of the mean. “About” can be understood as within 10%, 9%, 8%, 7%, 6%, 5%, 4%, 3%, 2%, 1%, 0.5%, 0.1%, 0.05%, or 0.01% of the stated value.

In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Unless otherwise clear from context, all numerical values provided herein are modified by the term “about.”

The term “administration” refers to introducing a substance into a subject. In general, any route of administration may be utilized including, for example, parenteral (e.g., intravenous), oral, topical, subcutaneous, peritoneal, intra-arterial, inhalation, vaginal, rectal, nasal, introduction into the cerebrospinal fluid, or instillation into body compartments. In some embodiments, administration is oral. Additionally or alternatively, in some embodiments, administration is parenteral. In some embodiments, administration is intravenous.

By “agent” is meant any small compound (e.g., small molecule), antibody, nucleic acid molecule, or polypeptide, or fragments thereof.

By “aprotic solvent” is meant a solvent that does not have an acidic hydrogen and cannot donate protons. Aprotic solvents may be polar or nonpolar and are characterized by their ability to dissolve ionic compounds while not participating in hydrogen bonding as proton donors. In the context of this disclosure, aprotic solvents may include dimethyl sulfoxide (DMSO), acetone, acetonitrile, dimethylformamide (DMF), tetrahydrofuran (THF), and similar compounds. In some embodiments, the aprotic solvent is dimethyl sulfoxide (DMSO), which may be used as a cryoprotectant for preserving cellular integrity during freezing and thawing processes.

By “control” or “reference” is meant a standard of comparison. In one aspect, as used herein, “changed as compared to a control” sample or subject is understood as having a level that is statistically different than a sample from a normal, untreated, or control sample. Control samples include, for example, cells in culture, one or more laboratory test animals, or one or more human subjects. Methods to select and test control samples are within the ability of those in the art. Determination of statistical significance is within the ability of those skilled in the art, e.g., the number of standard deviations from the mean that constitute a positive result.

By “disease endotype” is meant as a classification of a subtype of a disease condition (e.g., sepsis) and may refer to a subset of disease conditions with shared clinical or biological properties that may differ in prognosis, disease course, or therapeutic response. The disease endotype may be a sepsis-specific disease endotype. The sepsis-specific disease endotype may be a Molecular Diagnosis and Risk Stratification of Sepsis (MARS) endotype; or a Sepsis Response Signature (SRS) endotype; or selected from among the Neutrophilic-Suppressive (NPS), Inflammatory (INF), Innate Host Defence (IHD), Interferon (IFN), and Adaptive (ADA) endotypes, where the MARS endotype includes at least the set of MARS 1, MARS 2, MARS 3, MARS 4, and the SRS endotype includes at least the set of SRS 1 and SRS 2. In some embodiments, the sepsis-specific disease endotype is associated with neutrophil activation and immune suppression; associated with an increased pro-inflammatory response, e.g., increased NF-κB expression; associated with interleukin signaling; associated with increased IFN-α,β,γ; or associated with a variety of pathways including increased adaptive immunity.

By “marker” is meant any protein or polynucleotide having an alteration in expression level or activity that is associated with a disease or disorder.

As used herein, the term “subject” includes humans and mammals (e.g., mice, rats, pigs, cats, dogs, and horses). In many embodiments, subjects are mammals, particularly primates, especially humans. In some embodiments, subjects are livestock such as cattle, sheep, goats, cows, swine, and the like; poultry such as chickens, ducks, geese, turkeys, and the like; and domesticated animals particularly pets such as dogs and cats. In some embodiments, (e.g., particularly in research contexts) subject mammals may be, for example, rodents (e.g., mice, rats, hamsters), rabbits, primates, or swine such as inbred pigs and the like.

As used herein, the terms “treatment,” “treating,” “treat” and the like, refer to obtaining a desired pharmacologic and/or physiologic effect. The effect can be prophylactic in terms of completely or partially preventing a disease or symptom thereof and/or can be therapeutic in terms of a partial or complete cure for a disease and/or adverse effect attributable to the disease. “Treatment,” as used herein, covers any treatment of a disease or condition in a mammal, particularly in a human, and includes: (a) preventing the disease from occurring in a subject which can be predisposed to the disease but has not yet been diagnosed as having it; (b) inhibiting the disease, i.e., arresting its development; and (c) relieving the disease, i.e., causing regression of the disease. In some embodiments of the disclosure, the selection of treatment is determined based on a disease endotype. In some embodiments, the selection of a treatment is intended to avoid unnecessary and/or harmful treatment.

As used herein, “serum supplement” refers to a biological substance that is used as a supplement in cell cultures to help cells grow and maintain themselves. In some embodiments, the serum supplement is fetal bovine serum (FBS), newborn calf serum (NCS), horse serum, human serum, platelet lysate, bovine serum albumin (BSA), serum replacement, tryptose phosphate broth (TPB), insulin-transferrin-selenium (ITS), KnockOut™ Serum Replacement (KSR), CryoStor, or the like.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it is understood that the particular value forms another aspect. It is further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. It is also understood that throughout the application, data are provided in a number of different formats and that this data represent endpoints and starting points and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 as well as all intervening decimal values between the aforementioned integers such as, for example, 1.1, 1.2, 1.3, 1.4, 1.5, 1.6, 1.7, 1.8, and 1.9. With respect to sub-ranges, “nested sub-ranges” that extend from either end point of the range are specifically contemplated. For example, a nested sub-range of an exemplary range of 1 to 50 may comprise 1 to 10, 1 to 20, 1 to 30, and 1 to 40 in one direction, or 50 to 40, 50 to 30, 50 to 20, and 50 to 10 in the other direction.

The transitional term “comprising,” which is synonymous with “including,” “containing,” or “characterized by,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the present disclosure. Embodiments of this disclosure are described herein, including the best mode known to the inventors for carrying out the present disclosure. Variations of those embodiments may become apparent to those of ordinary skill in the art upon reading the foregoing description.

The disclosure illustratively described herein suitably can be practiced in the absence of any element or elements, limitation or limitations that are not specifically disclosed herein. Thus, for example, in each instance herein any of the terms “comprising”, “consisting essentially of”, and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the present disclosure. Thus, it should be understood that although the present disclosure provides preferred embodiments, optional features, modification and variation of the concepts herein disclosed may be resorted to by those skilled in the art, and that such modifications and variations are considered to be within the scope of this disclosure as defined by the description and the appended claims.

Other features and advantages of the present disclosure will be apparent from the following description of the preferred embodiments thereof, and from the claims. Unless otherwise defined, all technical and scientific terms used herein have the same meaning as commonly understood by one of ordinary skill in the art to which this present disclosure belongs. Although methods and materials similar or equivalent to those described herein can be used in the practice or testing of the present disclosure, suitable methods and materials are described below. All published foreign patents and patent applications cited herein are incorporated herein by reference. Genbank and NCBI submissions indicated by accession number cited herein are incorporated herein by reference. All other published references, documents, manuscripts and scientific literature cited herein are incorporated herein by reference. In the case of conflict, the present specification, including definitions, will control. In addition, the materials, methods, and examples are illustrative only and not intended to be limiting.

### BRIEF DESCRIPTION OF THE DRAWINGS

A better understanding of the features and advantages of the present disclosure may be obtained by reference to the following detailed description that sets forth illustrative embodiments, in which the principles of the disclosure are utilized, and the accompanying drawings below. The patent application file contains at least one drawing executed in color. Copies of this patent application publication with color drawing(s) will be provided by the Office upon request and payment of the necessary fee.

FIG. 1 shows an overview of sample processing methods: Ficoll and Cryo-PRO. Cryo-PRO is designed to expedite sample processing at the site of collection by incorporating a whole blood cryopreservation step (and subsequent red cell depletion step) to replace standard Ficoll processing, underlying an embodiment of the disclosure.

FIGS. 2A-2J show processing time and quality metrics by cryopreservation method. FIGS. 2A and 2B showing bar graphs, FIGS. 2C and 2D showing violin plots, FIG. 2E showing a plot, and FIGS. 2F, 2G, 2H, 2I, and 2J showing violin plots. For FIGS. 2F-2J, batches represent samples that were thawed, processed and sequenced together. Ficoll and Cryo-PRO samples from the same patient are next to each other. For patients where parallel processing occurred at both clinical sites (bottom row), the samples processed at the opposite site are shown in lighter shades. FIG. 2A shows “hands-on” time spent by operators at clinical sites to process patient samples from the start of processing after a blood draw to placing the sample in the freezer for storage. FIG. 2B shows the percent of CD45+ CD235a− CD15− cells staining DAPI negative on flow cytometry by method as an indicator of cell membrane integrity and cell viability. FIG. 2C shows violin plots of sequencing quality by method (left to right): unique genes per cell, unique molecular identifiers (UMIs) of RNA transcripts per cell, and percent of transcripts represented by mitochondrial genes per cell. FIG. 2D shows violin plots of CITE-seq quality metrics by method: unique surface protein features (left panel) and UMIs (right panel) for surface protein detection per cell (detected via CITE-seq). FIG. 2E shows number of singlet cells sequenced per method. Starting blood sample volume was variable in Ficoll samples and was 1 mL in Cryo-PRO samples. FIG. 2F shows per-sample violin plots of UMIs of RNA transcripts detected per cell. FIG. 2G shows per-sample violin plots of unique genes detected per cell. FIG. 2H shows per-sample violin plots of the percentage of mitochondrial transcripts per cell. FIG. 2I shows per-sample violin plots of unique surface protein features detected via CITE-seq per cell. A total of 137 different surface proteins were queried. FIG. 2J shows per-sample violin plots of UMIs of surface protein features detected via CITE-seq per cell.

FIGS. 3A-3F show a comparison of gene and protein profiling by method. FIG. 3A shows a projection, FIGS. 3B and 3C show dot plots, FIG. 3D shows a volcano plot, FIG. 3E shows a dot plot, and FIG. 3F shows a series of volcano plots. FIG. 3A shows a two-dimensional uniform manifold approximation and projection (UMAP) of cells by processing method; Ficoll (left) and Cryo-PRO (right). Dotted outlines represent major PBMC lineages. Cell substates were identified by clustering cells of each method independently; substate identities were then projected onto a shared set of UMAP axes (see Methods). FIG. 3B shows dot plots of representative marker genes and percent mitochondrial reads (percent.mt) for cell substates identified in scRNA-seq analysis by method (top: Ficoll, bottom: Cryo-PRO). Color represents scaled relative expression (blue=higher expression). Size represents the proportion of cells in each substate where the feature was detected. FIG. 3C shows dot plots of representative surface marker proteins (detected using CITE-seq) for each cell substate identified in scRNA-seq analysis by method (top: Ficoll, bottom: Cryo-PRO). Color represents scaled relative expression (blue=higher expression). Size represents the proportion of cells in each substate where the feature was detected. FIG. 3D shows a volcano plot of genes differentially up-regulated (positive Log 2FC) or down-regulated (negative Log 2FC) in Ficoll compared to Cryo-PRO after pseudobulking. Adjusted p-values of less than 0.05 are shown in red. Genes with p<0.05 and abs (Log 2FC)>1 are labeled. Plots are shown for differential gene expression among all cells. FIG. 3E shows dot plots of marker gene expression by each monocyte substate. FIG. 3F shows volcano plots of genes differentially up-regulated (positive Log 2FC) or down-regulated (negative Log 2FC) in Ficoll compared to Cryo-PRO after pseudobulking. Adjusted p-values of less than 0.05 are shown in red. Genes with p<0.05 and abs (Log 2FC)>1 are labeled. Plots are shown for differential gene expression among all cells (top left) and for each major cell type (subsequent plots).

FIGS. 4A-4I show trends in cell type and substate proportion by patient between method and processing center. FIGS. 4A, 4B, 4C, and 4D show scatter plots, FIGS. 4E and 4F show stacked bar charts. FIG. 4A shows scatter plots of cell type proportion from Ficoll and Cryo-PRO. Each cell type is represented by a different color and trendline. Proportion is the number of cells of one cell type divided by the total number of PBMCs from that patient sample. The patient-paired Ficoll: Cryo-PRO samples are plotted to assess correlation in method for each patient. Pearson's correlations (R) are shown for all correlations (*p<0.05, **p<0.01, ***p<0.001). FIG. 4B shows scatter plots of cell substate proportion from Ficoll and Cryo-PRO. Each cell substate is represented by a different color and trendline. Proportion is the number of cells of one cell substate divided by the total number of cells from its cell type from that patient sample. The patient-paired Ficoll: Cryo-PRO samples are plotted to assess correlation in method for each patient. FIG. 4C shows scatter plots of cell type proportion for samples processed at both locations, using Ficoll (left panel) or Cryo-PRO (right panel). Each point represents the proportion of one cell type from one patient sample, processed at each site. Each cell type is represented by a different color and trendline. Proportion is the number of cells of one cell type divided by the total number of PBMCs from that patient sample. The patient-paired Ficoll: Ficoll samples and Cryo-PRO: Cryo-PRO samples from the two different enrollment sites are plotted to assess variation in technical duplicates for each patient. Pearson's correlations (R) are shown for all correlations (*p<0.05, **p<0.01, ***p<0.001). FIG. 4D shows scatter plots of cell substate proportion from different processing sites. Each cell substate is represented by a different color and trendline. Proportion is the number of cells of one cell substate divided by the total number of cells from its cell type from that patient sample. The patient-paired Ficoll: Ficoll samples and Cryo-PRO: Cryo-PRO samples from the two different enrollment sites are plotted to assess correlation in method for each patient. Pearson's correlations (R, *p<0.05, **p<0.01, ***p<0.001) are shown for all correlations. For cell substates, correlations were significant for nearly all substates of monocytes, T cells, and B cells, and dendritic cells for each method between sites though for some substates including MS1, cross-site correlations were slightly lower for Cryo-PRO (right column) than Ficoll (left column). FIG. 4E shows cell substate proportions for technical duplicate samples processed at single centers. Samples from the same patient processed using different methods are shown next to each other. FIG. 4F shows cell substate proportions for technical duplicate samples processed at both centers. Samples from the same patient processed using different methods are shown next to each other; the corresponding pair of technical duplicates are shown subsequently. FIG. 4G shows a scatter plot of dendritic cell substate proportion from Ficoll and Cryo-PRO. FIG. 4H and FIG. 4I are graphs showing clonal expansion proportions for samples processed at single centers (A) and technical duplicate samples processed at both centers (B). Samples from the same patient processed using different methods are shown next to each other. In (B), the corresponding pair of technical duplicates processed at the non-origin site are shown subsequently, with the labeled site indicating where each sample was processed. PRO denotes Cryo-PRO.

FIGS. 5A-5F show that application of the Ficoll process after freezing and thawing whole blood samples is not effective due to red blood cell lysis leading to sample-to-sample variability. FIG. 5A illustrates the results of a direct-to-FACS method in which whole blood was mixed with DMSO, frozen, thawed, and then directly applied to FACS analysis to sort PBMCs. The top panel shows two heatmap images of PBMCs isolated after Ficoll treatment (left, top panel) and after the whole blood direct-to-FACS method described herein (right, top panel). The Ficoll plot highlights a population of PBMCs, with 98.20% of the sample falling within the designated gate. The whole blood direct-to-FACS plot shows the distribution of RBCs, with 17.29% of the sample within the selected gate. The lower graph shows uniform manifold approximation and projection (UMAP) representation of scRNA-seq data from PBMCs isolated by Ficoll and whole blood direct-to-FACS methods. FIG. 5B shows a bar graph of a comparative analysis of peripheral blood mononuclear cells (PBMC) recovery and viability across two experiments (Exp #1 and Exp #2) using either the whole blood direct-to-FACS method (dark bars) or the Ficoll method (light bars). Experiments that resulted in the flow cytometer being clogged are noted with a gray cloud. FIG. 5C outlines a process for isolating Peripheral Blood Mononuclear Cells (PBMCs) from patient blood samples for sequencing without addition of Ficoll (e.g., the whole blood direct-to-FACS method). The procedure begins with the collection of whole blood. Post-separation, the PBMC layer is frozen with an anticoagulant and DMSO at −140° C. After thawing, the cells undergo washing to remove debris and are stained for flow cytometry, ensuring only the desired cells are collected. The final step involves sorting the cells through flow cytometry, resulting in purified PBMCs ready for sequencing analysis. Prior art methods involving (1) density-gradient centrifugation with Ficoll pre-freeze, or (2) an experimental variation applying the Ficoll method post-thaw, are shown immediately above the shown procedure without Ficoll. FIG. 5D illustrates the impact of different sample preparation methods on the recovery and quality of Peripheral Blood Mononuclear Cells (PBMCs). Bar Graph (Top Left): Shows the number of viable PBMCs recovered using three different sample preparation methods: Pre-freeze Ficoll, whole blood direct-to-FACS, and Post-thaw Ficoll. Bar Graph (Top Right): Depicts the yield (% of cells sorted) and sorting time (minutes) for the same sample preparation methods. Post thaw Ficoll data is noted in dark bars, whole blood direct-to-FACS data is noted in medium gray bars, and experiments that resulted in the flow cytometer being clogged are noted with a gray cloud. The term “WB” listed under the medium gray bars refers to whole blood direct-to-FACS data. Ficoll data is shown in the light gray bars. Scatter Plots: Flow cytometry data showing cell populations Post-thaw Ficoll, whole blood direct-to-FACS, and Pre-freeze Ficoll treatments, labeled with CD235a vs. CD45 markers for patient 120-DO. Post-thaw Ficoll treatment improves PBMC yield compared to no Ficoll treatment. Post-thaw Ficoll treatment increases sorting time compared to no Ficoll treatment. FIG. 5E illustrates the impact of Post-thaw Ficoll treatment on the proportion of erythrocytes (CD235a+ cells) in peripheral blood samples. Flow Cytometry Scatter Plots: Left Plot: “Post Thaw Ficoll” shows the distribution of CD235a vs. CD45 markers in all cells from patient 120-DO. Center Plot: “Whole Blood (direct-to-FACS)” displays the same markers and cell populations without Ficoll treatment. Right Plot: “Post Thaw Ficoll (pre-Ficoll)” shows the distribution of CD235a vs. CD45 markers before Ficoll treatment. Bar Graph: Percentages of CD235a+ cells across three sample preparation methods: Post Thaw Ficoll, whole blood direct-to-FACS, and Ficoll. The proportion of cells staining positive for CD235a is lowered during the post-thaw Ficoll step compared to whole blood direct-to-FACS across all samples. FIG. 5F shows flow cytometry scatter plots comparing the expression of CD235a and CD45 on live cells (DAPI−). The data is categorized into three groups: “WB+Ficoll (post-thaw Ficoll),” “Whole Blood (direct-to-FACS),” and “Pre-freeze Ficoll (standard).”

FIGS. 6A-6C show that direct-to-flow-cytometer of blood samples frozen and subsequently thawing results in clogging of the flow cytometer. FIG. 6A presents data on the quantity and quality of Peripheral Blood Mononuclear Cells (PBMCs) in patient blood samples from the ARAMIS study, across different patients. Left Bar Graph: shows Essential FACS QC Metrics with number of cells sorted for patient numbers from 44 to 122 along with PBMC viability. Right Bar Graph: shows Additional FACS QC Metrics with % viable PBMCs after sorting for patient numbers consistent with the left graph along with sort time (min). Poor essential FACS metrics and cytometer clogging issues were encountered with this method. FIG. 6B presents the post-thaw hemocytometer cell counts for whole blood ARAMIS samples, comparing control and patient samples at two different time points: Baseline and 24 hours. The number of cells counted after thaw and first wash is on the lower range compared to MGH samples, which may explain some (but not all) of the poor FACS data. FIG. 6C shows flow cytometry scatter plots analyzing the presence of red blood cells (RBCs) and peripheral blood mononuclear cells (PBMCs) in different blood samples. P45—Baseline: Shows the distribution of CD235a (RBC marker) vs. CD45 (PBMC marker) in a baseline sample. P44—24 hrs: Displays the same markers in a sample taken 24 hours later. C3-86-D0 (Ficoll): Represents a sample processed with Ficoll. FIG. 6C indicates that clogging correlated with RBC overabundance in what was loaded onto the cytometer.

FIGS. 7A-7E show a comparison of four protocols (standard Ficoll; magnetic red blood cell (RBC) depletion also known as MACS—later incorporated into Cryo-PRO; freezing and thawing blood samples prior to Ficoll processing; and whole blood direct-to-flow-cytometer). FIG. 7A shows a schematic representation detailing the laboratory procedure for isolating and purifying peripheral blood mononuclear cells from whole blood samples using density gradient centrifugation, magnetic activated cell sorting with CD25a antibody, and fluorescence-activated cell sorting, followed by sequencing analysis. FIG. 7B shows a comparative analysis of four different methods for purifying Peripheral Blood Mononuclear Cells (PBMCs) from blood samples. The methods evaluated are Traditional Ficoll, Post-Thaw Ficoll, MACS (Magnetic Activated Cell Sorting)—later incorporated into Cryo-PRO, and Whole Blood. Data from four healthy controls are used to assess each method. The plots visually demonstrate the effectiveness of each method in terms of yield, viability, sort time, and purity. FIG. 7C presents a table showing a comparison of the efficiency of different PBMC purification techniques by showing the percentage yield of PBMCs and CD3a+5+ cells, the time taken for sorting these cells, and the number of cells sorted for each donor and method. The methods evaluated are Ficoll; MACS (Magnetic Activated Cell Sorting)—later incorporated into Cryo-PRO; Post-Thaw Ficoll; and WB (referring to whole blood direct-to-FACS). Data from four donors are used to assess each method. FIG. 7D shows a comparison of two methods of Peripheral Blood Mononuclear Cell (PBMC) purification: Traditional Ficoll and MACS (Magnetic-Activated Cell Sorting), later incorporated into Cryo-PRO. The analysis includes data from one healthy control and seven patients, with each patient's sample processed twice. Yield, viability, sort time, and purity of PBMCs is shown. FIG. 7E shows a table providing detailed experimental data on the efficiency and outcomes of different cell sorting methods (Traditional Ficoll and MACS (Magnetic-Activated Cell Sorting), later incorporated into Cryo-PRO) used on samples from various donors, highlighting the percentage yield, sort time, and number of cells sorted.

FIGS. 8A-8D show a comparison of the effectiveness of Ficoll and MACS (later incorporated into Cryo-PRO) methods for different cell populations. FIG. 8A shows two scatter plots comparing the effectiveness of Ficoll and MACS (Magnetic-Activated Cell Sorting, later incorporated into Cryo-PRO) methods for separating T cells marked by CD3 gene expression. The plots use Uniform Manifold Approximation and Projection (UMAP). FIG. 8B shows two scatter plots comparing the effectiveness of Ficoll and MACS (Magnetic-Activated Cell Sorting, later incorporated into Cryo-PRO) methods for separating B cells marked by the CD79A gene expression. The plots use Uniform Manifold Approximation and Projection (UMAP). FIG. 8C shows two scatter plots comparing the effectiveness of Ficoll and MACS (Magnetic-Activated Cell Sorting, later incorporated into Cryo-PRO) methods for separating NK cells marked by the GNLY gene expression. The plots use Uniform Manifold Approximation and Projection (UMAP). FIG. 8D shows two scatter plots comparing the effectiveness of Ficoll and MACS (Magnetic-Activated Cell Sorting, later incorporated into Cryo-PRO) methods for separating monocytes marked by the CD14 gene expression. The plots use Uniform Manifold Approximation and Projection (UMAP).

FIGS. 9A-9H show distribution of gene expression data specific to different cell populations. FIG. 9A shows a scatter plot of proportions of transcriptional substates of B- and T-lymphocytes for technical replicates of samples processed at each of two different clinical sites, showing comparably high correlations with Cryo-PRO (right panels) compared with standard Ficoll (left panels). FIG. 9B shows a volcano plot illustrating the distribution of gene expression data specific to MS1 cells. FIG. 9C shows a volcano plot illustrating the distribution of gene expression data specific to T cells. FIG. 9D shows a volcano plot illustrating the distribution of gene expression data specific to B cells. FIG. 9E shows a volcano plot illustrating the distribution of gene expression data specific to Natural killer cells. FIG. 9F shows a volcano plot illustrating the distribution of gene expression data specific to dendritic cells. FIG. 9G shows a volcano plot illustrating the distribution of gene expression data specific to monocytes. FIG. 9H shows a volcano plot illustrating the distribution of gene expression data for all cells.

FIG. 10A-10B show UMAP projections of Ficoll and Cryo-PRO T cells profiled and split by method. Cells are color coded for T cell substates (FIG. 10A) or by the number of identical TCR sequences that represent their clone size (FIG. 10B), respectively, where darker color equals greater clonal expansion.

FIG. 11A-11B show bar graphs depicting phagocytic activity as the fold-change in mean fluorescence intensity (MFI) of the pHrodo dye within live CD45+ CD15− cells, stratified by CD14 expression. FIG. 11A shows that on average, CD14+ cells exhibited a ˜4.5 fold (Ficoll), and ˜3.5 fold (Cryo-PRO) higher MFI than CD14-cells in the presence of the bioparticles. FIG. 11B shows the MFI of CD14+ cells from Ficoll was generally higher than CD14+ cells from the corresponding Cryo-PRO sample.

FIG. 12A-12D show a series of graphs providing a comparative analysis of identical TCR receptor clones detected by Cryo-PRO versus Ficoll methods from individual patients. FIG. 12A and FIG. 12B directly compare between Cryo-PRO (y-axis) and Ficoll (x-axis), while FIG. 12C and FIG. 12D compare across recruitment sites (BIDMC, y-axis; vs MGH, x-axis) for one method or the other (Ficoll or Cryo-PRO, indicated in each panel's title).

### DETAILED DESCRIPTION OF THE DISCLOSURE

The present disclosure is based, at least in part, on the unexpected discovery that direct cryopreservation of whole blood, followed by thawing and peripheral blood mononuclear cell (PMBC) isolation significantly reduces the time and technical expertise needed to obtain clinical samples, while still preserving single-cell transcriptomes and surface proteomes in patients. Accordingly, the present disclosure, in part, provides for the application of single-cell RNA sequencing (scRNA-seq) to complex clinical conditions across multiple collection sites, enabling better capture of the true heterogeneity of diseases. The present disclosure, in part, provides for a simplified whole blood cryopreservation method with PBMC recovery offsite. In certain aspects, methods of the present disclosure greatly reduce the time necessary to prepare samples. In certain aspects, methods of the present disclosure do not rely upon centrifugation. In some aspects, methods of the present disclosure include freezing whole blood samples with dimethyl sulfoxide (DMSO) in the absence of fetal bovine serum. In some aspects, methods of the present disclosure do not include positive selection of cells (e.g., use of a CD15+ or other cell marker). In some aspects, advantages of methods of the disclosure include, but are not limited to, reducing sample error and variability.

Single-cell RNA sequencing (scRNA-seq) of peripheral blood mononuclear cells (PBMCs) has enhanced understanding of host immune mechanisms in small cohorts, particularly in diseases with a complex and heterogeneous immune response to infection, such as sepsis. However, PBMC isolation from blood requires two hours of onsite processing using Ficoll density gradient separation (“Ficoll”) for scRNA-seq compatibility, precluding large-scale sample collection at most clinical sites. To eliminate complex onsite processing, the present disclosure provides for a Cryo-PRO (Cryopreservation with PBMC Recovery Offsite), a method of PBMC isolation from cryopreserved whole blood that allows immediate onsite sample cryopreservation and subsequent PBMC isolation in a central lab prior to sequencing. As shown in the following Examples, results from samples processed using Cryo-PRO versus standard onsite Ficoll separation in 23 sepsis patients and 1 healthy control were compared. Important scRNAseq outputs including cell substate fractions and representative marker genes were similar across multiple cell types and substates, including an important monocyte substate enriched in patients with sepsis (Pearson correlation 0.83, p<0.001; 87% of top marker genes shared). Cryo-PRO reduced onsite sample processing time from >2 hours to <15 minutes and was reproducible across two enrollment sites, thus demonstrating potential for expanding scRNA-seq in multicenter studies of sepsis and other diseases.

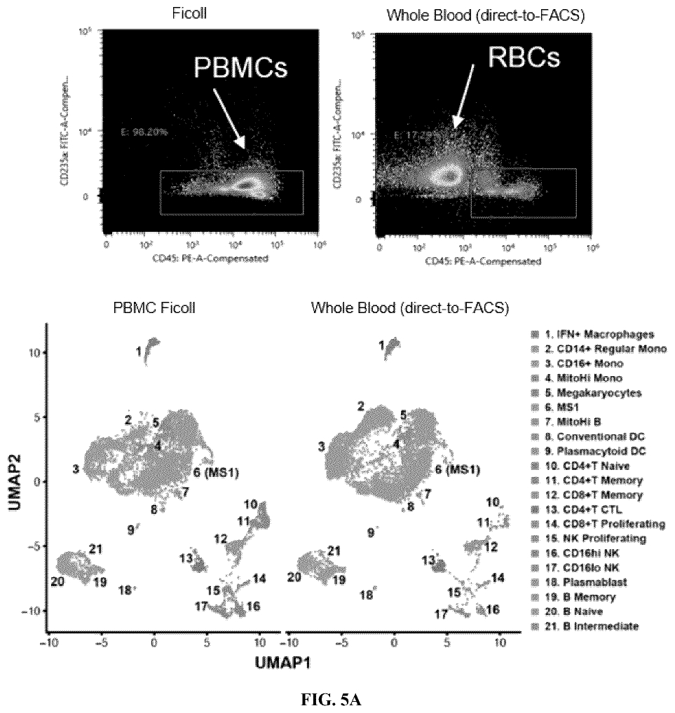

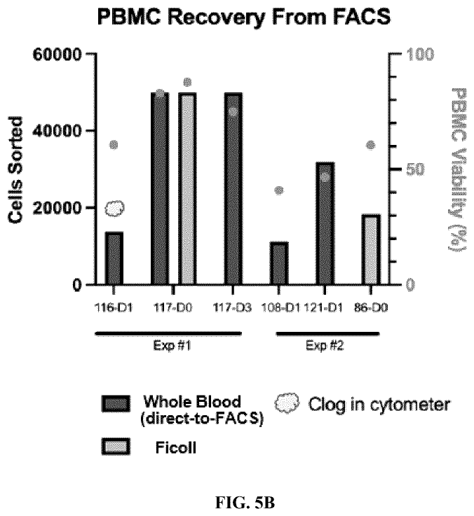

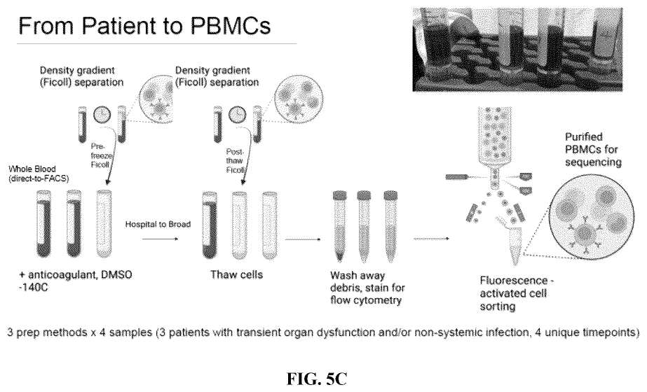

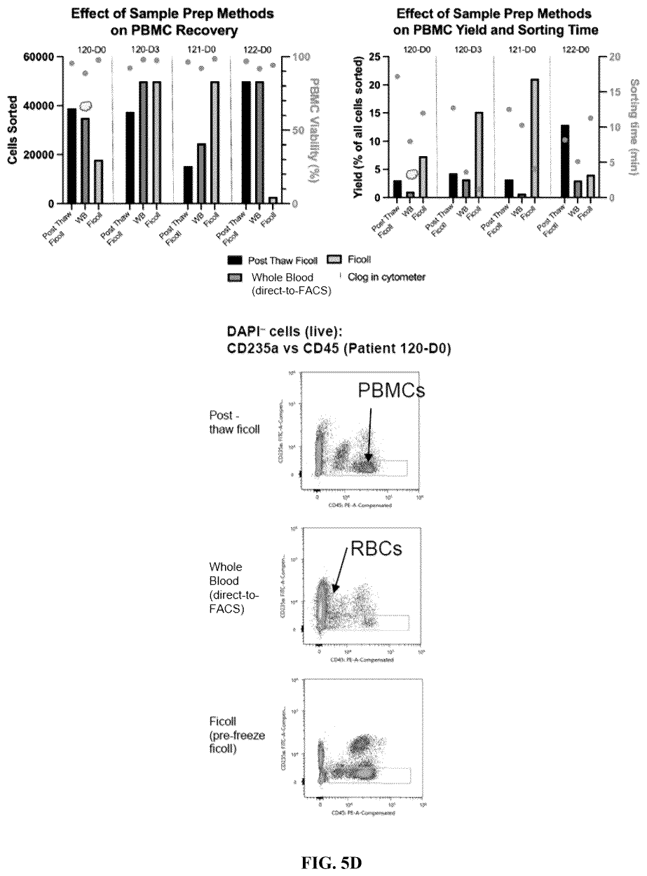

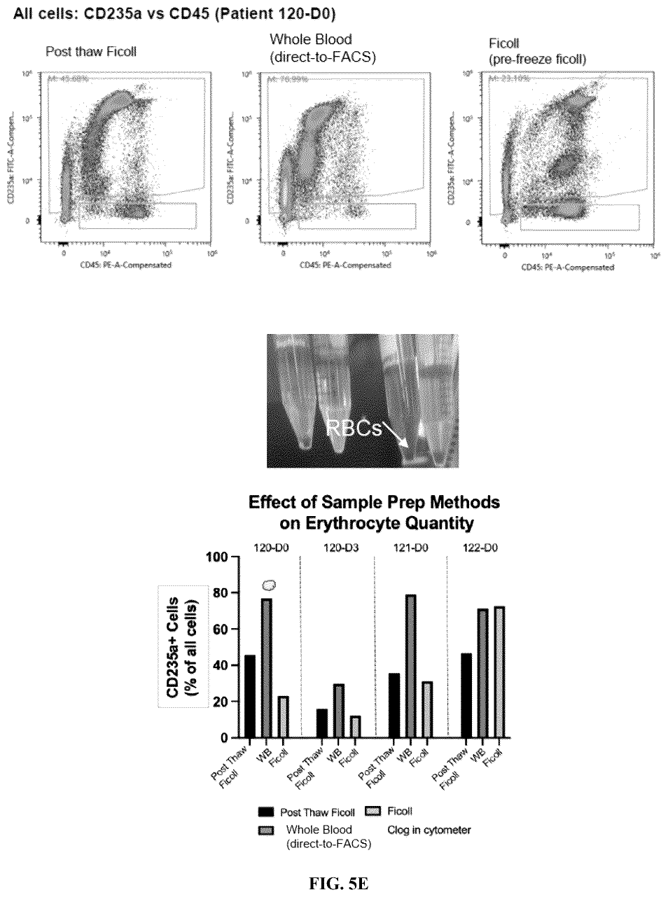

Sepsis is a life-threatening condition characterized by organ dysfunction resulting from a dysregulated host response to infection, with mortality rates ranging from 15% to 35%. Diagnosing sepsis remains challenging due to the non-specific nature of current diagnostic methods, which often fail to distinguish it from other inflammatory conditions. A novel approach proposed involves using MSI technology to effectively diagnose and treat sepsis by assessing immunosuppressive functions within immune cell populations. This method aims to improve the understanding of how these functions impact sepsis outcomes. By employing single-cell RNA sequencing of peripheral blood mononuclear cells, distinct molecular subtypes of sepsis, namely MSI1, MSI2, MSI3, and MSI4, can be identified. FIG. 5C includes a graphical abstract that visually represents these subtypes through an illustrated flow cytometry plot, highlighting the potential for more precise diagnostics and personalized treatment strategies. This information is crucial for developing new diagnostic methods or treatments targeting the specific molecular mechanisms involved in sepsis, offering significant advancements in medical research and patient care.

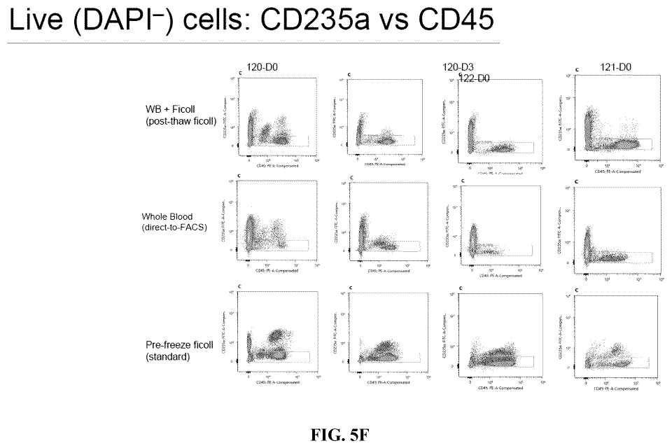

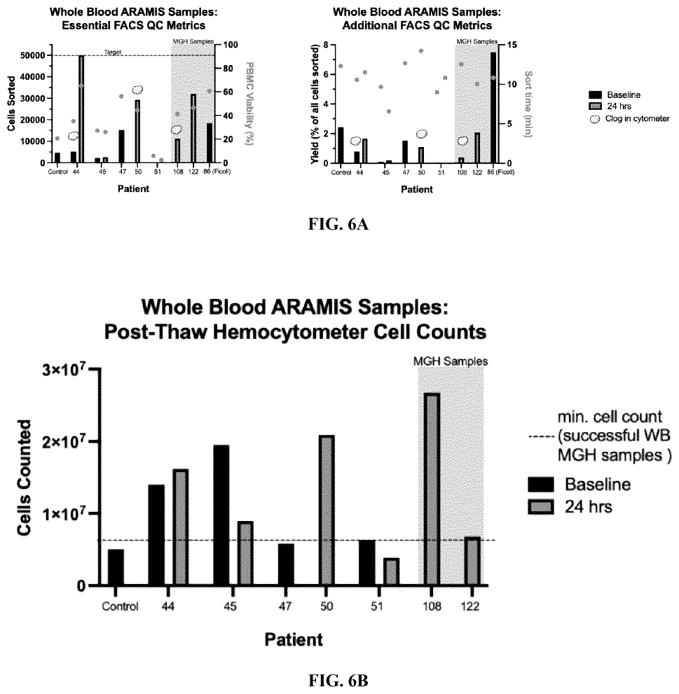

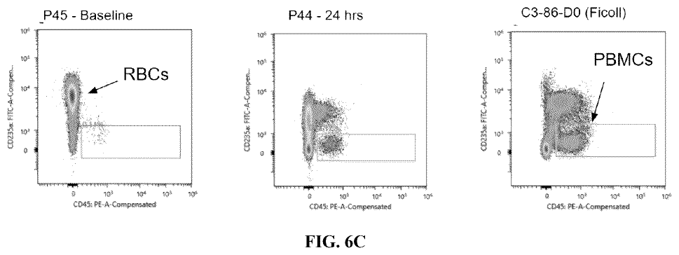

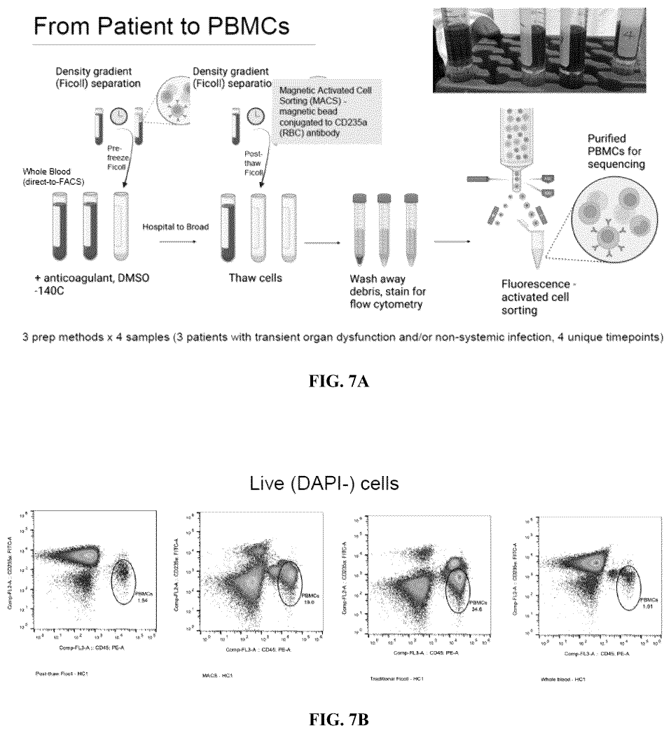

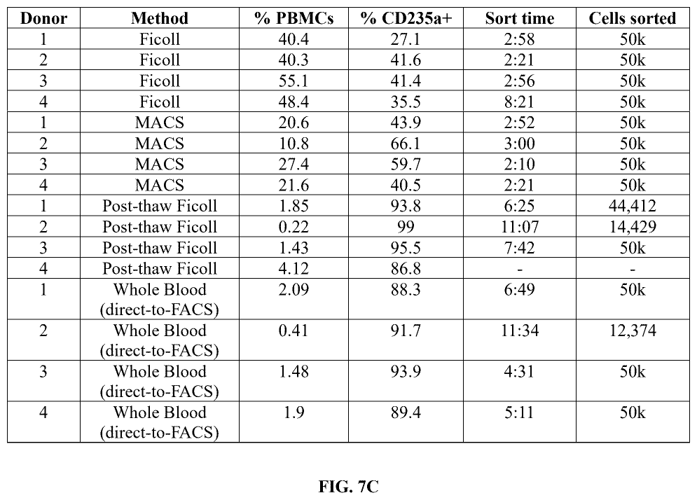

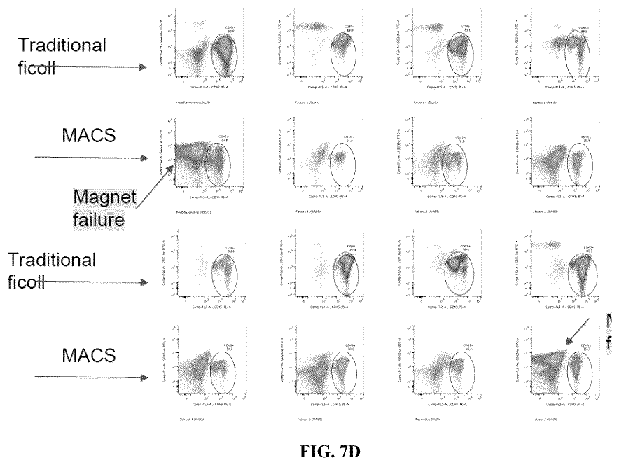

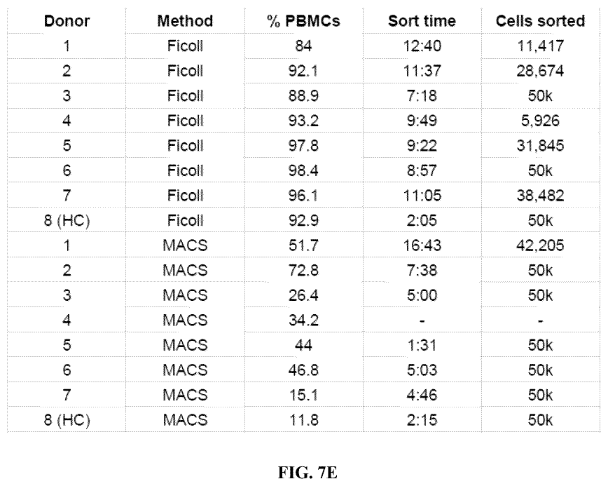

Implementing scRNA-seq studies in clinical settings is challenged by several logistical difficulties. Blood, which offers a diverse and dynamic snapshot of the systemic response to infection, serves as a key resource for investigating immune responses in sepsis and other conditions. However, since live blood cells are highly sensitive to environmental perturbations, it is necessary to either process samples rapidly before sequencing or employ cryostorage for later analysis. These steps help minimize any transcriptional changes in cells caused by stimuli after blood collection. Therefore, processing the blood sample to a point where transcription is halted (e.g., by freezing live cells or fixing them unless sequencing is performed immediately) often falls to operators at the sample collection site. Currently, scRNA-seq studies of PBMCs require a density gradient centrifugation step immediately following blood draw (Ficoll-paque processing, or “Ficoll”) to isolate and store immune cells. This process is resource-intensive, time-consuming, and sensitive to protocol variations. Additionally, the techniques herein showed that neither Ficoll processing nor flow cytometry may be adequately performed on cryopreserved samples, adding an additional barrier to the application of scRNA-seq on cryopreserved samples. Specifically, post-thaw Ficoll processing led to considerable sample-to-sample variability in cell recovery and purity (shown visually in the photo in FIG. 5E, and tabulated in FIG. 7C), and direct-to-FACS processing led to clogs in the FACS machine that significantly delayed sample processing (shown in FIG. 6A and by low recovery in 1 of 4 samples in FIG. 7C). Accordingly, the complexity of real-time processing of whole blood samples has limited the widespread use of scRNA-seq in clinical investigations. Moreover, the lack of standardization in processing and analysis can lead to batch effects, hindering comparisons across sites and between studies.

There are certain limitations to current methods for isolating peripheral blood mononuclear cells (PBMCs) using density gradient centrifugation (Ficoll). While Ficoll separation is a well-characterized process compatible with sequencing, it has significant drawbacks, including a 2-hour processing time, potential loss of 10% of blood samples due to deferred consent, operator variability, and batch effects that can affect transcriptional signatures. There is also need for technical expertise and specialized equipment. An improvement can be made in centralizing the processing of sepsis samples to expand single-cell RNA sequencing capabilities while maintaining high-quality standards. There is a need for developing a method to isolate PBMCs from whole blood after thawing to overcome these challenges and further improving PBMC isolation techniques for scalable and efficient single-cell RNA sequencing.

To overcome the practical limitations of scRNA-seq, the present disclosure provides for a Cryo-PRO (Cryopreservation with PBMC Recovery Offsite) method, a method for isolating PBMCs from cryopreserved whole blood samples. The approach utilizes magnetic depletion of red blood cells followed by fluorescence-activated cell sorting to recover immune cells for scRNA-seq. Cryo-PRO enables the immediate cryopreservation of whole blood samples at clinical sites, with onsite freezing and storage, allowing for their transfer at a later time to a centralized laboratory for PBMC isolation and scRNA-seq. As disclosed herein, the scRNA-seq output from sepsis patient samples processed using Cryo-PRO is compared with those processed by the standard onsite Ficoll-gradient separation method. These findings demonstrate technical equivalence and reproducibility between the two methods. Cryo-PRO can enable broad application of scRNA-seq to multicenter studies and clinical trials by simplifying sample collection and centralizing cell isolation to improve cost efficiency, minimize batch effects, and increase sample sizes. It has the potential to help improve the understanding of the complexity of sepsis and other heterogeneous diseases, enabling development of precision diagnostics and targeted therapeutic strategies.

In certain aspects, the present disclosure relates to heterogeneity of disease and the search for sepsis subtypes that relate to therapeutic response. Sepsis is a heterogeneous clinical syndrome with complex and variable underlying biological processes and cellular and metabolic derangements that lead to end-organ dysfunction. Heterogeneity manifests clinically as unexplained patient-level variability in disease trajectory, outcomes, and response to therapies, and it has been identified as a major reason for failed therapeutic trials in sepsis. Identification of relevant sepsis subtypes (also termed subphenotypes or endotypes) has thus been an emphasis of investigation. Clinical subphenotypes of sepsis have shown association with differential outcomes and treatment response; however, subjective and/or dynamic clinical attributes are challenging to translate into a real-time clinical tool to assign subphenotypes. Unbiased sepsis endotypes (based on host immune cell transcriptional signatures without clinical data) have been derived from bulk RNA-sequencing (RNA-seq) data that also demonstrate differential mortality and treatment effects. However, bulk RNA-seq signatures may largely reflect the predominant circulating cell type (e.g., neutrophils), and important discriminators of underlying biological mechanisms (cell type- and subtype-specific gene programs) can be masked.

There exists a need for broad availability of scRNA-seq to clarify disease heterogeneity and define targets for precision diagnostics and therapeutics. Single-cell transcriptional profiling (scRNA-seq) has transformed the appreciation for heterogeneity in circulating immune cells. Developed over the past decade, scRNA-seq has been employed for translational research primarily in oncology and inflammatory diseases. A unique monocyte subtype (MS1, characterized by immunosuppressive gene program) that is expanded in sepsis vs infection without sepsis and that likely plays a role in the development and progression of sepsis has been identified. Sentinel papers highlight single-cell profiling in elucidating immune cell gene programs, including MS1, in severe COVID-19 that provide insight into mechanisms of disease. It is expected that much of the clinical heterogeneity observed in disease trajectory, outcomes, and treatment response in sepsis may be reflected in cell-type specific transcriptional heterogeneity in circulating immune cells. Harnessing scRNA-seq, the method of choice to characterize cellular heterogeneity, may uniquely improve the understanding of the complexity of sepsis, enabling development of precision diagnostics and targeted therapeutic strategies.

Yet to date, cohorts are small and scRNA-seq has not been widely employed in clinical investigation, in part due to the complexity of real-time processing of whole blood samples needed to isolate immune cells. Further, the lack of standardization in processing and analysis creates batch effects, hindering comparisons across sites and between studies. The heterogeneity of sepsis demands large-scale multicenter clinical investigations utilizing scRNA-seq, both for mechanistic characterization of the immune response and for evaluating therapeutic interventions. The field would benefit from standardization and simplification of methods for generating and analyzing such data in order to meaningfully harness the power of single-cell immune profiling to better understand sepsis. To address this important need, the present disclosure provides a simplified process for collection and preservation of samples from clinical settings while centralizing single-cell processing, sequencing, and analysis of scRNA-seq data, and rigorously validates these methods. It is expected that in scaling up to five clinical sites, methods of the present disclosure may provide a streamlined approach to scRNA-seq to 170 patients with sepsis, to derive and analyze scRNA-seq-based endotypes.

Although cryopreservation has been used after the laborious process of isolating tissue or immune cells, such as Ficoll gradient centrifugation for peripheral blood mononuclear cells (PBMCs), direct cryopreservation of whole blood for later thawing and deep immune profiling with scRNA-seq and CITE-seq has not been previously demonstrated. Unlike the current gold standard of PBMC Ficoll separation, which takes 2 hours and considerable technical expertise, cryopreservation of whole blood requires only minutes and no specialized equipment. Thus, whole blood cryopreservation allows for broad application to multicenter collection efforts and clinical trials where resources and expertise are not available for detailed cell separation procedures and analytical methods, which could be centralized to specialized facilities. It also minimizes variation in processing that can lead to batch effects, particularly across different clinical sites. Aspects of the present disclosure optimize this approach; enabling deep immune profiling of diverse patient cohorts at multiple clinical sites, greatly facilitating deep study of the immune response to disease and therapies.

Optimized whole blood cryopreservation methods and techniques disclosed herein may be expanded across 5 academic medical centers that actively collaborate in large, multicenter clinical trials for sepsis. This may allow a platform to demonstrate the application of immediate whole blood cryopreservation of collected samples with centralized immune cell separation and scRNA-seq at a center of expertise. This may in turn enable the following novel investigations: 1) derive de novo scRNA-seq-based endotypes on a large, diverse cohort of sepsis patients of varied geographic and demographic makeup; 2) directly compare scRNA-seq-derived endotypes with bulk RNA sequencing data obtained from the same sample, enabling precise characterization of immune cell signatures and cell-specific gene programs that underpin bulk RNA-seq-derived endotypes; and 3) apply scRNA-seq-derived endotypes to publicly available bulk RNA sequencing data from published clinical trials to elucidate endotype-specific treatment effects and cell-specific gene programs that might influence these effects.

Application of single-cell host immune profiling to multicenter observational studies and randomized-controlled trials (RCTs) is needed to better understand immune mechanisms involved in sepsis and explain variability in treatment effects and outcomes amongst patients with sepsis. Deployed at scale, scRNA-seq and associated technologies such as CITE-seq are ready to fill the need for deep immune profiling to reveal the cellular heterogeneity that is hypothesized to provide new insights into patient-level heterogeneity in sepsis. Equally important are functional and mechanistic studies on paired samples to test hypothesized function of discovered immune cell subtypes and sepsis-specific scRNA-seq-derived endotypes. However, the current gold standard for onsite sample processing for scRNA-seq is complex, time consuming, and highly sensitive to variations in protocol, and thus not ideal for deployment across multiple hospitals to support large-scale clinical studies that have personnel with varying levels of technical expertise. There is a crucial opportunity to integrate single-cell profiling into large observational studies and RCTs and integrate single-cell profiling with biomarker, proteomic, metabolomic, and bulk transcriptomic analyses, as well as facilitate associated mechanistic studies.

To meet this need, the present disclosure provides for the development, optimization, and validation of a new sample processing method that greatly simplifies on-site protocols for scRNA-seq, transferring the technically demanding steps to a centralized location to enable scale-up for multi-hospital sepsis investigations. The core tenet of the presently disclosed method involves rapid cryopreservation of fresh whole blood samples to replace the time-intensive and technically-involved standard procedures for clinical site immune cell isolation. It has been demonstrated that dimethyl sulfoxide (DMSO) cryopreservation of whole blood samples preserves lymphocyte viability. Another study demonstrated preserved viability, compared with fresh samples, of lymphocytes and myelocytes separated, immunolabeled, and cryopreserved for varying amounts of time, with subsequent analysis by flow cytometry. Intracellular cytokines were also detectable post-cryopreservation via intracellular immunolabeling in cells that were LPS-stimulated prior to cryopreservation. DMSO has been used to cryopreserve PBMCs separated from whole blood for later scRNA-seq, and 10% DMSO has been employed in standard PBMC Ficoll separation protocols with success. Most recently, different studies of cryopreservation in tissue cells found DMSO to be superior to alternative methods in producing high-quality scRNA-seq results. However, each of these studies still involved a technically demanding step prior to cryopreservation that would be difficult to standardize across multiple study sites. The techniques herein provide the ability to optimize a simple, streamlined approach to onsite sample processing that is compatible with key downstream analyses, including scRNA-seq and functional immune profiling, thereby enabling large scale, multicenter sepsis studies.

Single-cell transcriptional profiling facilitates high-resolution characterization of the heterogeneity among circulating immune cells, thereby revealing important insights into diseases such as sepsis where the immune response plays a pivotal role. Performing these investigations with clinical samples is important for translational goals, as it establishes a direct link between cellular transcriptomics and patient-derived data. However, the current state-of-the-art process for scRNA-seq faces a number of major roadblocks to application on clinical samples: intensive sample collection strategies that require more time, equipment, and molecular techniques than are typically available to clinical study teams; and cost. ScRNA-seq is becoming more economical with emerging technologies and the ability to pool samples, but performing scRNA-seq from patient blood still requires PBMC isolation via the time- and resource-intensive process of Ficoll density gradient centrifugation. This limitation has constrained the application of scRNA-seq in clinical investigations resulting in smaller clinical cohorts that may not fully capture the heterogeneity of diseases under study.

Here, the present disclosure demonstrates that direct cryopreservation of a small volume (˜1 mL) of whole blood at the point of care, followed by thawing and PBMC isolation at a centralized research facility, is a viable alternative to on-site Ficoll processing for scRNA-seq and CITE-seq. This simple and streamlined approach significantly reduced the time and technical expertise needed to obtain clinical samples, while still preserving single-cell transcriptomes and surface proteomes in patients with sepsis. As described herein, the same immune cell types and substates in the datasets of Cryo-PRO and Ficoll are identified, including the sepsis-enriched monocyte substate MS1, which is considered to be important in sepsis immunopathophysiology. These data show a high correlation between methods (Cryo-PRO and Ficoll) for the abundances of all major cell types. Although cell substates may be less distinctly defined by their transcriptional profile than cell types and are therefore more susceptible to misidentification due to stochasticity in clustering, high correlations between most substate proportions derived from the two methods after independent clustering and substate assignment were still observed. Moreover, similar patterns of gene and surface protein expression across cell types and substates with very minimal differential gene expression between methods were observed. TCR capture was successful from T cells processed with Cryo-PRO, with sequences and proportions of expanded clonotypes similar to those of Ficoll. Together, this substantial equivalence between the gold-standard method of Ficoll processing and Cryo-PRO demonstrates that Cryo-PRO does not introduce major artifacts from processing and generates results with biological significance in patients. When deployed across two different enrolling emergency departments, cell type and substate abundances from Cryo-PRO showed strong correlations across sites. This finding shows that Cryo-PRO is robust to variations in collection site and operator, further validating it as a reliable strategy for expanding scRNA-seq studies.

An exemplary protocol for isolating Peripheral Blood Mononuclear Cells (PBMCs) from patient blood samples for sequencing is disclosed herein. The process begins with the collection of whole blood, which is then mixed with an anticoagulant and DMSO at −140° C. The sample undergoes density gradient separation using Ficoll to isolate the PBMC layer, while plasma and platelets are removed. The PBMCs are then frozen and transported to a facility. Upon arrival, the cells are thawed, washed to remove debris, and stained for flow cytometry. Finally, fluorescence-activated cell sorting (FACS) is used to purify the PBMCs for sequencing. This method is referred to as direct-to-FACS sorting. As noted above, this method has a tendency to clog FACS machinery.

A whole blood (WB) cryopreservation technique that simplifies the isolation of peripheral blood mononuclear cells (PBMCs) uses DMSO as a cryoprotectant, eliminating the need for Ficoll. This method leverages flow cytometry to analyze various cell types, including CD45+ leukocytes, CD3+ T cells, CD19+ B cells, and CD235a− erythrocytes, while excluding dead cells. Key findings include over 90% PBMC viability and comparable sequencing quality to traditional methods, with around 50,000 PBMCs sorted using FACS. These results demonstrate the method's efficiency and potential for high-quality cell preservation and analysis, making it a valuable innovation for medical research and diagnostics.

Assessing sample quality prior to fluorescence-activated cell sorting (FACS) of peripheral blood mononuclear cells (PBMCs) is crucial. The variability between experiments and patients can be influenced by differences in sample preparation. For accurate comparisons, it is crucial to match samples by patient and timepoint between whole blood (WB) and Ficoll-prepared samples. Additionally, WB samples may require erythrocyte depletion before sorting to prevent cell aggregation and clogging, ensuring the reliability and validity of FACS results. This information is important for standardizing procedures in patient applications related to cell sorting technologies, as it addresses important pre-sorting quality checks that impact performance.

Another exemplary protocol for isolating Peripheral Blood Mononuclear Cells (PBMCs) from patient blood samples for sequencing is disclosed herein. The process begins with the collection of whole blood, which is then subjected to density gradient separation using Ficoll. The PBMCs are then frozen with an anticoagulant and DMSO at −140° C. and transported to a facility labeled ‘Broad.’ Upon arrival, the cells are thawed, washed to remove debris, and stained for flow cytometry. Finally, fluorescence-activated cell sorting (FACS) is employed to purify the PBMCs for sequencing. The process can further comprise a pre-freeze Ficoll step, or a post-thaw Ficoll step.

Essential FACS metrics during fluorescence-activated cell sorting (FACS) of peripheral blood mononuclear cells (PBMCs), specifically viability and total PBMCs sorted, were poor. These issues may be patient-specific, influenced by factors such as sample preparation and sit times. Additionally, cytometer clogging was observed in MGH and ARAMIS whole blood samples. The number of cells counted after thawing and the first wash is lower compared to MGH samples, which may partially explain the poor Fluorescence-Activated Cell Sorting (FACS) data. Unsuccessful samples have a high proportion of red blood cells compared to peripheral blood mononuclear cells (PBMCs).

By greatly simplifying on-site protocols for scRNA-seq and transferring the technically demanding steps to a centralized location, the Cryo-PRO method has transformative potential for multicenter sample collection and clinical trial enrollment efforts. The resource demands of onsite processing for scRNA-seq particularly impacts studies of highly heterogeneous diseases with acute onset where study collection strategies are ideally deployable at any time a patient may present. Sepsis is an archetype of such a condition, and sample sizes for scRNA-seq studies of sepsis have, as a consequence, been too small to bring the full power of the method to bear on investigating biological reasons underlying the clinical heterogeneity of the condition. The substantial time reduction in sample processing and preservation (i.e., mean time of 13 minutes for Cryo-PRO vs 143 minutes for Ficoll) has crucial operational implications in the clinical research setting. Simplifying sample collection also offers an opportunity for improving cost efficiency by enabling the rapid enrollment of many potentially suitable patients for clinical studies, followed by retrospective adjudication to inform the selection of appropriate patients for sequencing. Widening the net of subjects enrolled in this manner better reflects the true patient heterogeneity in conditions under study. For sepsis, this strategy facilitates the derivation of scRNA-seq-based endotypes on a large, diverse cohort of sepsis patients with varied clinical presentations and demographic backgrounds, including those from health centers in underserved communities without dedicated research teams and resources to typically participate in clinical research.

Other forms of rapid whole blood cryopreservation have recently been demonstrated with scRNA-seq. In one of these studies, a substantial loss in the fractional abundance of myeloid cells was observed when compared with samples obtained using Ficoll. The present method produces better equivalence with the standard Ficoll method across immune cell types. Another method is based on the use of fixed cells, which provides more flexibility in the cryopreservation process compared to Ficoll. However, because fixation impairs polymerases involved in cDNA library preparation, fixed cell RNA profiling requires hybridization to a predefined set of probes, rather than sequencing, to detect transcripts, introducing a number of limitations. In particular, hybridization-based approaches require a priori knowledge of the cell's potential transcriptional signature, and thus fail to capture regions of high allelic diversity such as TCR (and B cell receptor) clonotypes. Other options for rapid sample processing and preservation with fixed cell profiling are generally kit-specific, requiring users to commit to an approach prior to the start of sample collection and use costly reagents for all collected samples, and sacrifice the potential for diverse allele region capture before experimentation even begins. The approach disclosed herein enables kit-agnostic preservation to simplify and expedite sample collection, preserving the option to later profile diverse allele regions such as TCR clonotypes. Lastly, a disadvantage of prior art fixed cell profiling is that it necessitates killing the cells. Cryo-PRO was found to leave cells alive and capable of performing phagocytosis, suggesting that cells retain their phenotype and are still responsive to environmental stimuli. Many sequencing studies require such active cellular functions, for example, measuring cells' transcriptional responses to stimuli or Perturb-seq. These prior approaches, in part due to smaller sample size, relied on co-clustering of scRNA-seq data with the traditional Ficoll method to assign cell states. In order to be useful at the point of care, any streamlined collection method must stand alone; the techniques herein independently clustered and analyzed patient-matched data from Cryo-PRO alone, versus Ficoll alone, and found substantial technical and biological equivalence.

The present disclosure (n=24 subjects and 32 paired samples) is the largest to date evaluating the feasibility of whole blood cryopreservation for scRNA-seq and CITE-seq in any context, and demonstrates substantial equivalence with conventional methods. The techniques herein provide for use of Cryo-PRO as a sample processing approach in a larger cohort of subjects. Second, although all major cell types and substates had substantial equivalence in patient-level abundance, some cell substate abundances deviated between Cryo-PRO and Ficoll methods. Some differences in substate assignment within cell types (e.g., MS1 versus classical CD14+ monocytes or memory versus naive B cells) are less well-defined than differences in cell types, and may reflect more of a continuum than a dichotomy, so more stochastic differences in assignments are expected. Other cell substates like gamma delta T cells and plasmablasts were present at very low abundances and therefore were more susceptible to outlier effects. While some differences between methods may reflect differences in either gene expression or survival by cell type and substate, each method introduces processing steps that may perturb transcription, i.e., centrifugation for 2 hours through a density gradient followed by freezing, thawing, and flow cytometry for Ficoll; exposure to DMSO, freezing, thawing, magnetic cell separation, and flow cytometry for Cryo-PRO. The overall agreement between methods suggests that major transcriptional signals that reflect biology are likely preserved. The techniques disclosed herein provide for assessment of function of PBMCs isolated by Cryo-PRO, whereas Ficoll preparation is known to yield functional PBMCs, enabling correlation of transcriptional states with cellular activity. As described herein, the techniques provide for assessment of the functional capacity of PBMCs isolated using the Cryo-PRO method.

By greatly simplifying sample collection at the point of care, Cryo-PRO unlocks the potential of scRNA-seq to study the biology of complex clinical conditions across multiple collection sites, including lower-resource settings, thus enabling better capture of the true heterogeneity of diseases. This method greatly lowers the barrier to embedding scRNA-seq-compatible collection strategies in randomized clinical trials, which enables post-hoc analyses to identify biological subsets of patients (i.e., endotypes) who may selectively respond to therapeutic interventions. In addition, Cryo-PRO could enhance the cost-efficiency of scRNA-seq by enabling “overcollection” at the point of care, reserving PBMC isolation and scRNA-seq only for samples from patients who display clinical phenotypes or disease trajectories of interest on subsequent adjudication. Thus, Cryo-PRO substantially expands the application of scRNA-seq towards personalized medicine in complex and heterogeneous conditions like sepsis, and this work represents an important step towards that goal.

Sepsis, identified by the World Health Organization (WHO) as a global health priority, has no proven pharmacologic treatment other than appropriate antibiotic agents, fluids, vasopressors as needed, and possibly corticosteroids (Venkatesh, B., Finfer, S., Cohen, J., Rajbhandari, D., Arabi, Y., Bellomo, R., Billot, L., Correa, M., Glass, P., Harward, M., et al. (2018). Adjunctive Glucocorticoid Therapy in Patients with Septic Shock. N. Engl. J. Med. 378, 797-808). Thus, current treatment for sepsis includes: (i) the administration of antibiotics and, where indicated, surgical or interventional radiological approaches for eliminating or at least controlling the source of infection; (ii) the administration of intravenous fluids (Lactated Ringer's solution; crystalloid solutions such as 0.9% sodium chloride solution, or colloid solutions such as 5% albumin solution) to restore and maintain adequate intravascular volume; (iii) the infusion of titratable vasoconstricting and/or inotropic drugs, such as vasopressin or noradrenaline, as needed, to change the strength of a heart's contractions; and/or, when indicated, (iv) mechanical ventilation, various forms of renal replacement therapy and, in rare cases, venovenous or venoarterial extracorporeal membrane oxygenation.

### Kits

It is contemplated within the scope of the disclosure that the techniques herein may be provided in the form of kits for cryopreserving and processing whole blood for single-cell RNA sequencing. Such kits may comprise dimethyl sulfoxide (DMSO), a buffer comprising phosphate buffered saline (PBS), ethylenediaminetetraacetic acid (EDTA), and a serum supplement, a red blood cell depletion reagent, and instructions for use. The kits may be configured as single-use or multi-sample formats, with component volumes ranging from about 1 mL to about 100 mL depending on the intended sample volume and throughput.

Kit components may be stored at temperatures ranging from about −20° C. to about 25° C., with shelf lives of up to about 24 months. The DMSO may be provided in concentrations suitable for creating final mixtures of between about 5% and about 15% v/v when combined with whole blood samples. Buffer components may be provided as separate reagents or as pre-mixed solutions, with EDTA concentrations of between about 1 mM and about 5 mM and serum supplement concentrations of between about 1% and about 5% v/v.

The kits may further include quality control components including positive and negative control samples, viability assessment reagents, and reference standards for validating scRNA-seq performance. Flow cytometry reagents may include fluorescently labeled antibodies against CD45, CD235a, CD15, and additional markers for cell identification and sorting. The kits may also include cryovials, storage containers, and specialized packaging materials designed to maintain component stability during shipping and storage.

Instructions provided with the kits may include detailed protocols with timing specifications, troubleshooting guides, equipment requirements, safety precautions, and data analysis workflows. The instructions may specify compatibility with various blood collection tubes, automated processing equipment, and different scRNA-seq platforms. The kits may be configured for research-grade or clinical-grade applications, with appropriate quality control measures and regulatory compliance features specific for the desired application.

For diagnostic applications, the kits may additionally comprise reagents and instructions for identifying sepsis-specific disease endotypes, including guidance for recognizing MARS, SRS, NPS, INF, IHD, IFN, and ADA endotypes. Treatment selection guidance based on identified endotypes may also be included. The kits may be designed to process sample volumes ranging from about 0.5 mL to about 20 mL of whole blood, with expected PBMC recovery rates of at least about 70% and viability rates of at least about 90%.

### EXAMPLES

### Example 1: Methods

### Sex as a Biological Variable.

Eight female patients and fifteen male patients were included in the study, in addition to one male healthy control subject. To account for patient heterogeneity, including potential effects of sex as a biological variable, all comparisons were made by comparing samples processed with different methods but collected from the same subject.

### Patient Enrollment and Clinical Adjudication

This study was conducted at Massachusetts General Hospital and Beth Israel Deaconess Medical Center. Inclusion criteria were adult patients arriving at the Emergency Department with evidence of organ dysfunction for whom bacterial infection was possible or suspected. Eligible patients had a blood sample collected under an IRB-approved alteration of informed consent, which allowed a research sample to be drawn simultaneously with the initial clinical blood draw. Informed consent was obtained from the patient or a surrogate at a later time after initial resuscitation.

Samples were collected for 100 patients during a 12-month period from April 2023 to March 2024. Of those 100, consent to analyze sample for research was obtained in 84 patients, thus considered enrolled. Sample was discarded for those who did not provide consent. Clinical data were collected on all enrolled subjects and entered into REDCap by clinical research coordinators. Physician adjudication (MRF) was later performed via retrospective chart review with access to all available clinical data and notes during the subject's hospitalization. Subjects were adjudicated as meeting Sepsis-3 criteria for sepsis or septic shock during the first 48 hours of hospitalization, or whether infection without sepsis versus other non-infectious cause for presenting illness was present. For the current analysis, sequencing in those subjects adjudicated as sepsis and septic shock was prioritized. 23 subjects were selected to be sequenced and included in the analysis.

### Sample Collection

Research blood samples were collected in 10 mL EDTA tubes. Up to 20 mL was collected if patient samples were being parallel-processed at both clinical sites; up to 10 mL was collected if patient samples were being processed at only a single site. For samples parallel-processed at both clinical sites, one of the two 10 mL EDTA tubes collected at the enrolling site was couriered to the second site. This resulted in a delay in processing of about 2 hours on average; samples from one subject were delayed >3 hours. Samples obtained for single-site processing were taken directly to the onsite lab for immediate processing. Processing of all 10 mL EDTA samples involved cryopreservation of whole blood (2 mL) and onsite density gradient centrifugation with Ficoll (˜3 to 6 mL) as described below. Up to 3 mL of the collected whole blood sample was used for other research purposes.

### Cryo-PRO Whole Blood Cryostorage

For immediate whole blood cryopreservation, 2.0 mL of blood from the 10 mL EDTA tube were mixed with 200 uL DMSO. Two 1-mL aliquots in cryovials were then prepared per sample and were slowly cooled using a Mr Frosty (Sigma-Aldrich) in a −80° C. freezer. Aliquots were stored onsite at −80° C. for less than 1 month before being transported to the Broad Institute (Cambridge, MA) on dry ice and immediately stored at −140° C. until the time of sequencing. Two 1 mL aliquots were cryopreserved in order to have a backup sample if needed.

### Ficoll Cryostorage

Density gradient centrifugation was performed on the remaining blood in the EDTA tubes (˜3 to 6 mL). Blood with EDTA was diluted 1:1 with room temperature PBS and layered over Ficoll-Paque PLUS density gradient media (Cytivia) in a SepMate tube (STEMCELL) before centrifuging at 1,200 rcf for 20 minutes at 20° C. with slow acceleration and the brake off. The buffy coat layer was carefully collected and washed twice with cold RPMI (Gibco) before cells were counted, resuspended in CryoStor CS10 (STEMCELL), and aliquoted into cryotubes targeting 1 million cells per vial. Samples were cooled, stored, and transported in the same manner as the Cryo-PRO samples.

### Processing Center Comparison

For a subset of samples, two tubes of blood (up to 20 mL) were collected from a patient at one Emergency Department. One tube remained onsite, while the other tube was immediately couriered to the other participating medical center. Upon receipt of the sample at the other medical center, both centers simultaneously began independent processing and cryostorage of the patient samples as described above, by both Cryo-PRO and Ficoll methods at each site. As before, processing and cryopreservation began within 4 hours of sample collection.

### Healthy Donor Blood Cryostorage

Fresh healthy donor blood in EDTA tubes was ordered from Research Blood Components (Watertown, MA) and processed within two hours of receipt. Whole blood and PBMC cryostorage steps took place as described, though all processing steps occurred at the Broad Institute.

### Pre-Sequencing Processing

On the day of flow cytometry sorting and Chromium 10× processing, a sample of cryopreserved whole blood (for Cryo-PRO) and a Ficoll sample were thawed for each patient. Sequencing batches were designed to contain four Ficoll samples and four patient-matched Cryo-PRO samples to minimize the effect of sequencing batch variation on the method comparison; therefore, 8 samples total were processed in parallel.

For each of the four Cryo-PRO samples, 1 mL of cryopreserved whole blood was thawed in a 37° C. water bath for 1 min 15 seconds and transferred into a 5-mL polystyrene round-bottom tube using 1 mL of PBS containing 2 mM EDTA and 2.5% FBS. Samples were immediately depleted of red blood cells using the STEMCELL EasySep RBC depletion kit. Briefly, the diluted blood was mixed with 50 μL of the RBC depletion reagent before immediately being placed on a magnet for 5 minutes at room temperature. The supernatant was pipetted off and mixed with an additional 50 μL of RBC depletion reagent in a new tube before another immediate 5 minute magnet incubation. At the end of the second incubation, the supernatant was transferred into 8.5 mL of FBS-RPMI (RPMI+10% FBS+1× penicillin/streptomycin) on ice. These steps were performed in parallel for the four Cryo-PRO samples.

For each of the four Ficoll samples, one vial per patient was thawed in a 37° C. water bath for 1 min 15 seconds before transfer with 1 mL of FBS-RPMI into 8.5 mL of FBS-RPMI on ice. For patients with three or more Ficoll vials, two vials were thawed and combined to improve cell recovery. These steps were performed in parallel for the four Ficoll samples.

For the subsequent steps, Cryo-PRO and Ficoll samples received the same treatment and steps were performed in parallel. All samples were centrifuged to pellet the cells (300×g, 5 minutes, 4° C.), then resuspended with FACS-PBS (PBS+2 mM EDTA+2.5% FBS) and centrifuged again. Each sample was then resuspended in 50 uL FACS-PBS and incubated on ice with a hashtag oligo for pooled sequencing (TotalSeq™ anti-human Hashtags, BioLegend), an Fc receptor blocking solution (Human TruStain FcX™, BioLegend), and flow cytometry stains (DAPI solution, Thermo Scientific; Alexa Fluor® 700 anti-human CD15 [Clone: H198], BioLegend; FITC anti-human CD235a [Clone: HI264], BioLegend; and PE anti-human CD45 [Clone: HI30], BioLegend). Samples were then washed in cold FACS-PBS and sorted on a SONY MA800 cell sorter to select for DAPI− CD15− CD235a− CD45+ cells, with a sorting target of 50,000 cells per sample.

After sorting, the hashed and sorted cells from all eight samples were pooled, pelleted (300×g, 5 minutes, 4° C. in FACS-PBS), and resuspended in a CITE-Seq cocktail for surface proteome measurement for a final incubation on ice. After 20 minutes, the cells were washed twice more (centrifugation at 300×g, 5 minutes, 4° C. followed by resuspension in PBS+2.5% FBS), counted, and resuspended in PBS+2.5% FBS for a target concentration of 1,000 cells/uL.

Library Construction and scRNA Sequencing

Droplet-based single-cell RNA capture and RNA library construction was performed with the Chromium single-cell 5′ kit v2 (10× Genomics, Inc). Forty uL of cells were loaded onto the Chromium Chip K, and Gel Bead-in Emulsion creation and library construction followed according to the manufacturer's protocol.

Eight batches of libraries were prepared (including gene expression libraries and cell surface protein libraries), with each batch barcoded using the 10× Dual Index Kit and sequenced altogether. Gene expression libraries were sequenced at a low depth (˜200 reads/cell) using the MiniSeq 150 Cycle Hi-Output Kit (Illumina) for a quality check and cell count estimate to inform library balancing. Rebalanced libraries targeting 50,000 reads/cell for gene expression and 10,000 reads/cell for surface proteins were then sequenced on an Illumina NovaSeq S4.

### Data Preprocessing

FASTQ files were aligned to a reference genome (GRCh38) using the Cell Ranger v6 pipeline by 10× Genomics. Demultiplexing and multiplet detection with patient hashtag oligos was performed using the Cumulus pipeline. Filtered gene expression matrices and CITE-Seq matrices were then analyzed using the Seurat V5 package in R. Multiplets and cell barcodes without corresponding gene expression, CITE-seq, and demultiplexing data were removed. Genes present in less than 10 cells were removed. Sequencing data from each method was split into two datasets and analyzed independently. For each set, RNA expression data was normalized, scaled, and integrated between sequencing batches using the top 2,000 most variable genes. Scaled CITE-seq data was integrated by finding multimodal neighbors using the first 50 principal components of RNA and CITE-seq data.

### Clustering and Substate Identification

Clustering was performed using the resulting weighted-nearest-neighbors graph, and the Clustree package was used to determine clustering resolution. Cell types were assigned to clusters using top marker genes for each cluster (determined by Wilcoxon rank-sum test, Bonferroni-corrected p-value<0.05, ranked by fold-change), and cell substates were assigned using top marker genes obtained by subsetting and re-clustering cells from each cell type at a higher resolution. Classification of cell types and substates was cross-referenced using the annotated Azimuth reference dataset. Clusters were defined as low quality if over 20% of cells in the cluster were cells with mitochondrial genes representing 10% or more of total genes detected in that cell. Low quality clusters were removed from further analysis as part of an extended quality control. After method-independent cell substate assignment, the Ficoll and Cryo-PRO datasets were combined and a UMAP was generated using the weighted-nearest-neighbors graph for the purpose of data visualization.

### Differential Gene Expression

To assess differential gene expression between methods, scRNA-seq data was first pseudo-bulked by sample (generating 32 “bulk” RNA-seq samples from each method) to minimize p-value inflation, and FindMarkers with the DESeq2 package was used to detect differentially expressed genes. The same process occurred for differential gene expression at the cell type level, although cells were first pseudo-bulked by cell type in addition to sample.

Top marker genes for each cell substate were calculated in Seurat using the FindMarkers function, and genes with an expression log fold-change>0.25, genes expressed in over 25% of cells in the cluster; and a Bonferroni-corrected p-value<0.05 were included.

### Cell Substate Abundance

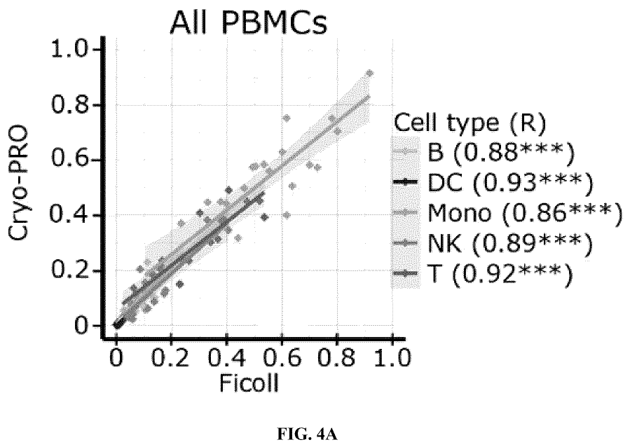

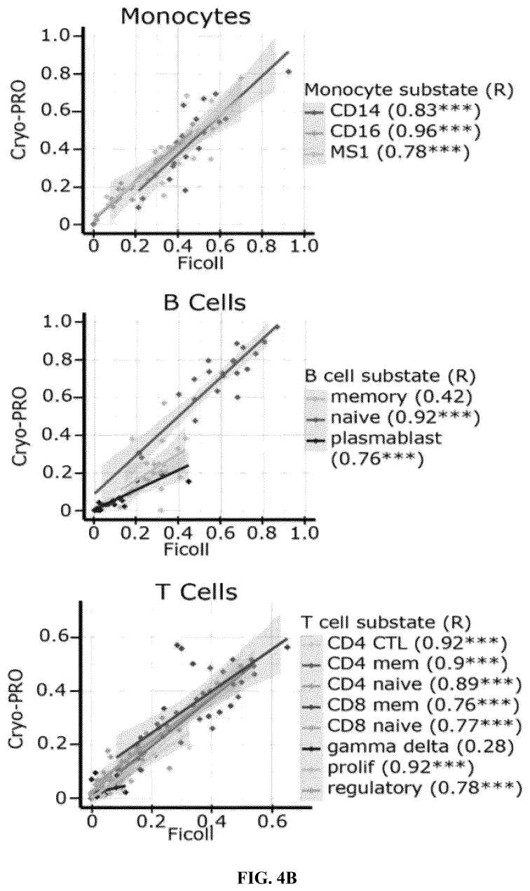

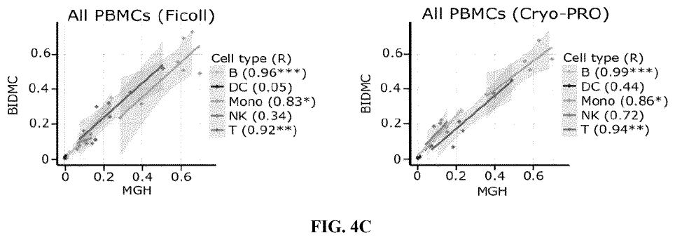

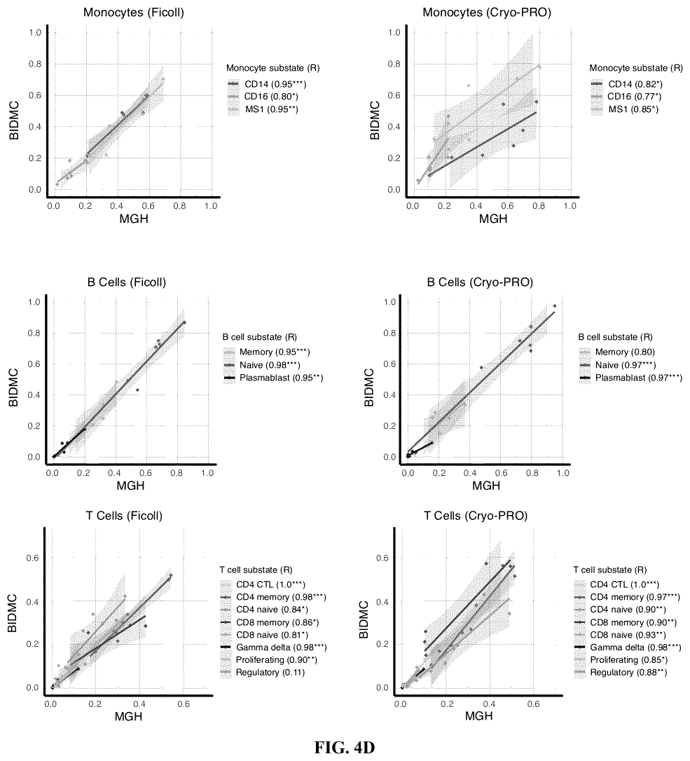

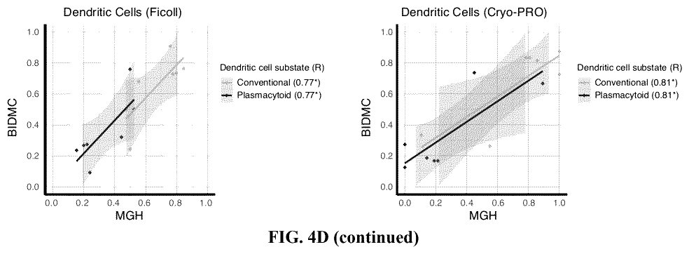

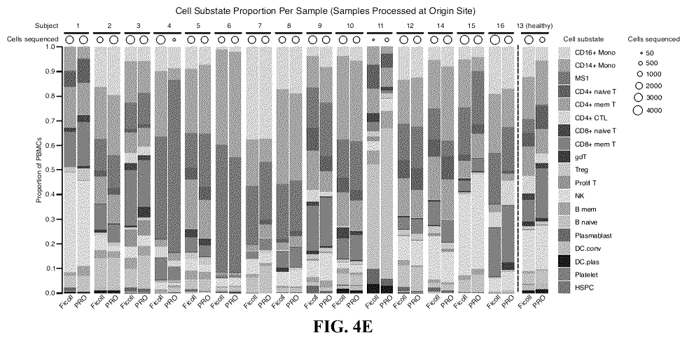

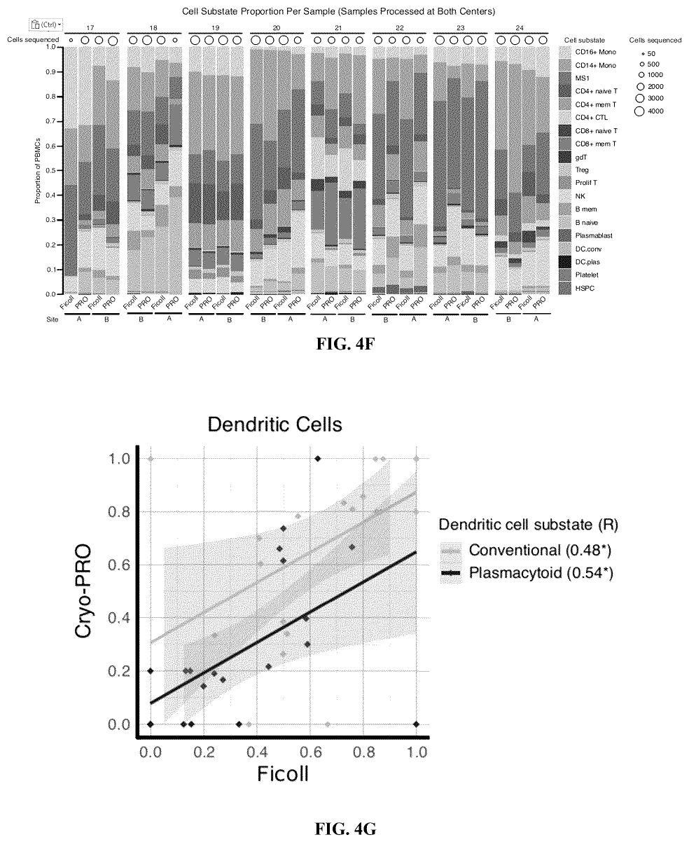

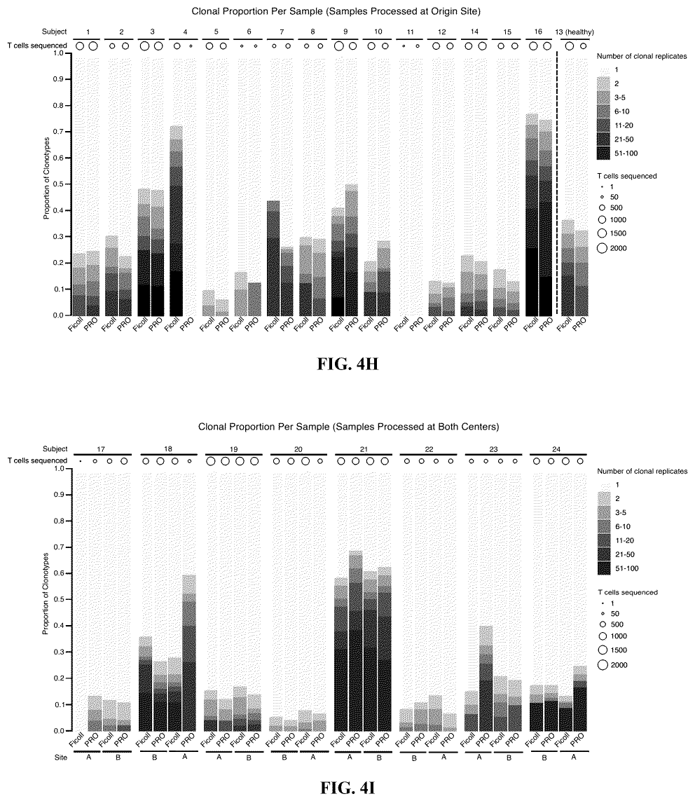

PBMC cell type proportions were calculated as a fraction of all major cell types identified (monocytes, B cells, T cells, NK cells, and dendritic cells). Cell substate proportions were calculated as a fraction of the cell type in question. Cell clusters defined as low quality, or belonging to a class of cells other than PBMCs, were not included in proportion calculations. Samples with fewer than 1,000 cells were not included in correlation calculations or in FIG. 4E, FIG. 4F due to effects of low sample sizes. When possible, the Ficoll-Cryo-PRO comparisons were made using samples processed at the same site they were collected at. In the case of BI-04, one of these samples had below 1000 cells, so the samples processed at the alternative site were used in calculations instead. R values were calculated for the scatterplots of cell types and substates shown using a Pearson correlation. Slopes and 90% confidence intervals for all trendlines were calculated by fitting a linear regression model to each cell type and substate in R.

### Data Visualization

Figures were generated using the ggplot2 package, the ScCustomize package, and the Seurat package in R.

### TOR Sequencing and Repertoire Analysis.

Paired α/β TCR sequences were obtained using the 10× Genomics 5′ V(D)J Immune Profiling workflow. Following single-cell capture and cDNA amplification, TCR libraries were constructed in parallel with gene expression libraries from the same droplets, according to the manufacturer's protocol. Libraries were sequenced to sufficient depth to recover full-length V(D)J transcripts. TCR reads were processed using Cell Ranger pipelines to assemble CDR3 sequences for both TCRα and TCRβ chains. Cells lacking a productive TCR sequence were excluded. Productive paired TCRαβ chains were extracted using the combineTCR( ) function in the scRepertoire R package. Clonotypes were defined by identical CDR3 amino acid sequences for both α and β chains, and clonal expansion was visualized using scRepertoire functions.

### Phagocytosis Assays

Samples were collected and stored as described above. Sample thaw followed steps in pre-sequencing processing through the first wash in FACS-PBS.

### Figure Generation

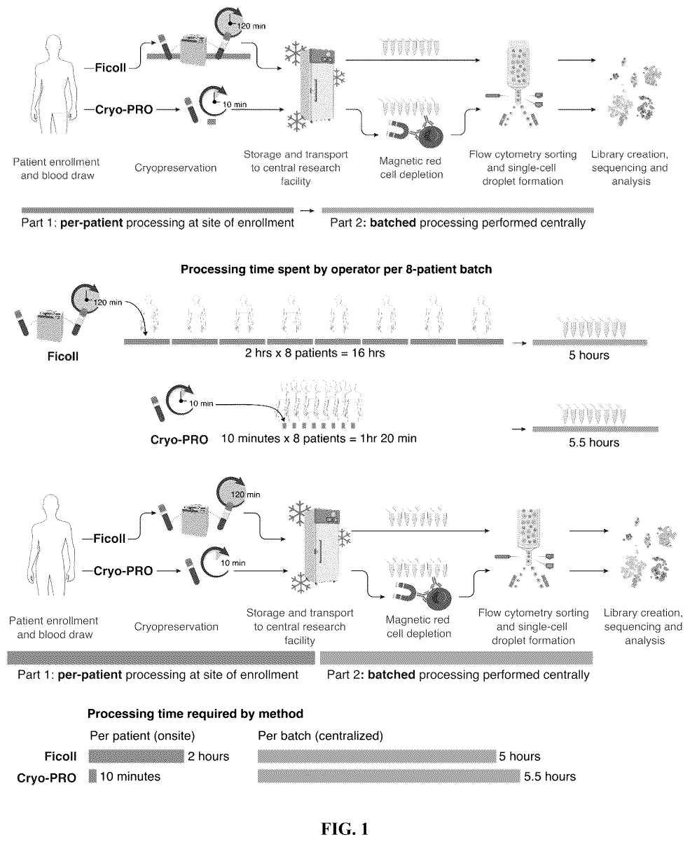

FIG. 1 was created in BioRender. Subsequent figures were generated in R using the ggplot2 package, the ScCustomize package, the Seurat package, and the scRepertoire package.

Study approval.

This study was approved by the Massachusetts General Brigham IRB (2022P002833). Eligible patients had a blood sample collected under an IRB-approved alteration of informed consent, which allowed a research sample to be drawn simultaneously with the initial clinical blood draw. Informed consent was obtained from the patient or a surrogate at a later time after initial resuscitation.

### Comparison of Techniques

In some aspects, the presently disclosed methods were developed based on the finding that application of the Ficoll process after freezing and thawing whole blood samples is not effective due to red blood cell lysis leading to sample-to-sample variability (FIG. 5A-F). For example, FIG. 5A illustrates the results of a direct-to-FACS method in which whole blood was mixed with DMSO, frozen, thawed, and then directly applied to FACS analysis to sort PBMCs. The top panel shows two heatmap images of PBMCs isolated after Ficoll treatment (left, top panel) and after direct-to-FACS method described herein (right, top panel). The Ficoll plot highlights a population of PBMCs, with 98.20% of the sample falling within the designated gate. The whole blood direct-to-FACS plot shows the distribution of RBCs, with 17.29% of the sample within the selected gate. The lower graph shows uniform manifold approximation and projection (UMAP) representation of scRNA-seq data from PBMCs isolated by Ficoll and whole blood direct-to-FACS methods. FIG. 5B shows a bar graph of a comparative analysis of peripheral blood mononuclear cells (PBMC) recovery and viability across two experiments (Exp #1 and Exp #2) using either the whole blood direct-to-FACS method (dark bars) or the Ficoll method (light bars). FIG. 5C outlines a process for isolating Peripheral Blood Mononuclear Cells (PBMCs) from patient blood samples for sequencing without addition of Ficoll. The procedure begins with the collection of whole blood. Post-separation, the PBMC layer is frozen with an anticoagulant and DMSO at −140° C. After thawing, the cells undergo washing to remove debris and are stained for flow cytometry, ensuring only the desired cells are collected. The final step involves sorting the cells through flow cytometry, resulting in purified PBMCs ready for sequencing analysis. Prior methods involving (1) density-gradient centrifugation with Ficoll pre-freeze, or (2) and experimental variation applying the Ficoll method post-thaw, are shown immediately above the shown procedure without Ficoll. FIG. 5D illustrates the impact of different sample preparation methods on the recovery and quality of Peripheral Blood Mononuclear Cells (PBMCs). Bar Graph (Top Left): Shows the number of viable PBMCs recovered using three different sample preparation methods: Pre-freeze Ficoll, whole blood direct-to-FACS, and Post-Thaw Ficoll. Bar Graph (Top Right): Depicts the yield (% of cells sorted) and sorting time (minutes) for the same sample preparation methods. Post-thaw Ficoll data is noted in dark bars, whole blood direct-to-FACS data is noted in light bars, and experiments that resulted in the flow cytometer being clogged are noted with a gray cloud. The term “WB” listed under the medium gray bars refers to whole blood direct-to-FACS data. Ficoll data is shown in the light gray bars. Scatter Plots: Flow cytometry data showing cell populations Post-thaw Ficoll, whole blood direct-to-FACS, and Pre-freeze Ficoll treatments, labeled with CD235a vs. CD45 markers for patient 120-DO. Post-thaw Ficoll treatment improves PBMC yield compared to no Ficoll treatment. Post-thaw Ficoll treatment increases sorting time compared to no Ficoll treatment. FIG. 5E illustrates the impact of Post-thaw Ficoll treatment on the proportion of erythrocytes (CD235a+ cells) in peripheral blood samples. Flow Cytometry Scatter Plots: Left Plot: “Post Thaw Ficoll” shows the distribution of CD235a vs. CD45 markers in all cells from patient 120-DO. Center Plot: “Whole Blood (direct-to-FACS)” displays the same markers and cell populations without Ficoll treatment. Right Plot: “Post Thaw Ficoll (pre-Ficoll)” shows the distribution of CD235a vs. CD45 markers before Ficoll treatment. Bar Graph: Percentages of CD235a+ cells across three sample preparation methods: Post Thaw Ficoll, whole blood direct-to-FACS, and Ficoll. The proportion of cells staining positive for CD235a is lowered during the post-thaw Ficoll step compared to whole blood direct-to-FACS across all samples. FIG. 5F shows flow cytometry scatter plots comparing the expression of CD235a and CD45 on live cells (DAPI−). The data is categorized into three groups: “WB+Ficoll (post-thaw Ficoll),” “Whole Blood (direct-to-FACS),” and “Pre-freeze Ficoll (standard)”.

It was further found that direct-to-flow-cytometer of blood samples frozen and subsequently thawing results in clogging of the flow cytometer (FIG. 6A-C). FIG. 6A presents data on the quantity and quality of Peripheral Blood Mononuclear Cells (PBMCs) in patient blood samples from the ARAMIS study, across different patients. Left Bar Graph: shows Essential FACS QC Metrics with number of cells sorted for patient numbers from 44 to 122 along with PBMC viability. Right Bar Graph: shows Additional FACS QC Metrics with % viable PBMCs after sorting for patient numbers consistent with the left graph along with sort time (min). Poor essential FACS metrics and cytometer clogging issues were encountered with this method. FIG. 6B presents the post-thaw hemocytometer cell counts for whole blood ARAMIS samples, comparing control and patient samples at two different time points: Baseline and 24 hours. The number of cells counted after thaw and first wash is on the lower range compared to MGH samples, which may explain some (but not all) of the poor FACS data. FIG. 6C shows flow cytometry scatter plots analyzing the presence of red blood cells (RBCs) and peripheral blood mononuclear cells (PBMCs) in different blood samples. P45-Baseline: Shows the distribution of CD235a (RBC marker) vs. CD45 (PBMC marker) in a baseline sample. P44-24 hrs: Displays the same markers in a sample taken 24 hours later. C3-86-D0 (Ficoll): Represents a sample processed with Ficoll. FIG. 6C indicates that clogging correlated with RBC overabundance in what was loaded onto the cytometer.

Through comparison with a variety of techniques (standard Ficoll; magnetic red blood cell (RBC) depletion also known as MACS, later incorporated into Cryo-PRO; freezing and thawing blood samples prior to Ficoll processing; and whole blood direct-to-flow-cytometer) it was determined that magnetic RBC depletion was suitable (FIG. 7A-E). FIG. 7A shows a schematic representation detailing the laboratory procedure for isolating and purifying peripheral blood mononuclear cells from whole blood samples using density gradient centrifugation, magnetic activated cell sorting with CD25a antibody, and fluorescence-activated cell sorting, followed by sequencing analysis. FIG. 7B shows a comparative analysis of four different methods for purifying Peripheral Blood Mononuclear Cells (PBMCs) from blood samples. The methods evaluated are Traditional Ficoll, Post-Thaw Ficoll, MACS (Magnetic Activated Cell Sorting)—later incorporated into Cryo-PRO, and Whole Blood. Data from four healthy controls are used to assess each method. The plots visually demonstrate the effectiveness of each method in terms of yield, viability, sort time, and purity. FIG. 7C presents a table showing a comparison of the efficiency of different PBMC purification techniques by showing the percentage yield of PBMCs and CD3a+5+ cells, the time taken for sorting these cells, and the number of cells sorted for each donor and method. The methods evaluated are Ficoll, MACS (Magnetic Activated Cell Sorting)—later incorporated into Cryo-PRO, Post-Thaw Ficoll, and Whole Blood (WB). Data from four donors are used to assess each method. FIG. 7D shows a comparison of two methods of Peripheral Blood Mononuclear Cell (PBMC) purification: Traditional Ficoll and MACS (Magnetic-Activated Cell Sorting), later incorporated into Cryo-PRO. The analysis includes data from one healthy control and seven patients, with each patient's sample processed twice. Yield, viability, sort time, and purity of PBMCs is shown. FIG. 7E shows a table providing detailed experimental data on the efficiency and outcomes of different cell sorting methods (Traditional Ficoll and MACS (Magnetic-Activated Cell Sorting), later incorporated into Cryo-PRO) used on samples from various donors, highlighting the percentage yield, sort time, and number of cells sorted.

It should be appreciated that a person of ordinary skill in the art could adapt methods of the present disclosure to use with a high-throughput magnet to allow for the processing of 8-16 samples at once. In some aspects, the present methods allow for isolation of PBMCs and depletion of red blood cells expressing CD235. Prior to any purification step, RBCs start as >95% of cells in whole blood, whereas PBMCs start as <1%. Thus, in certain aspects flow cytometry improves the isolation of PMBCs, but that RBC depletion is important to avoid clogging of the flow cytometer.

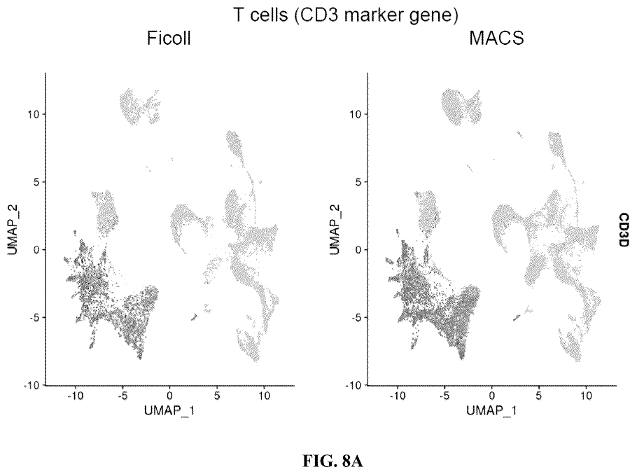

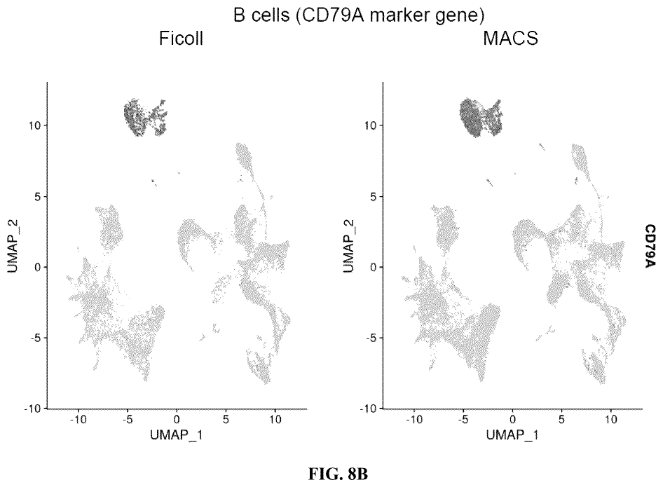

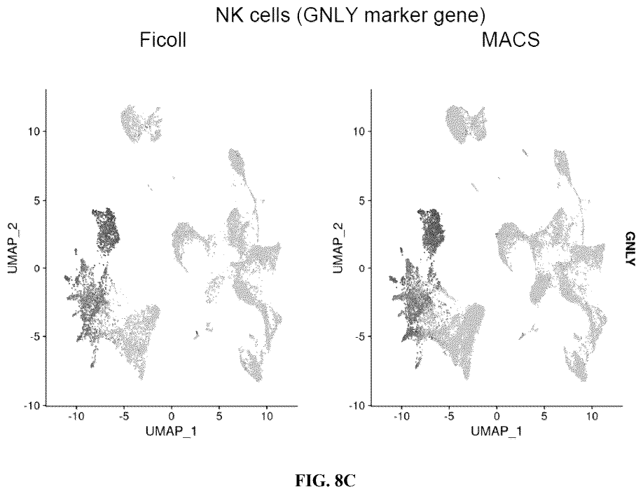

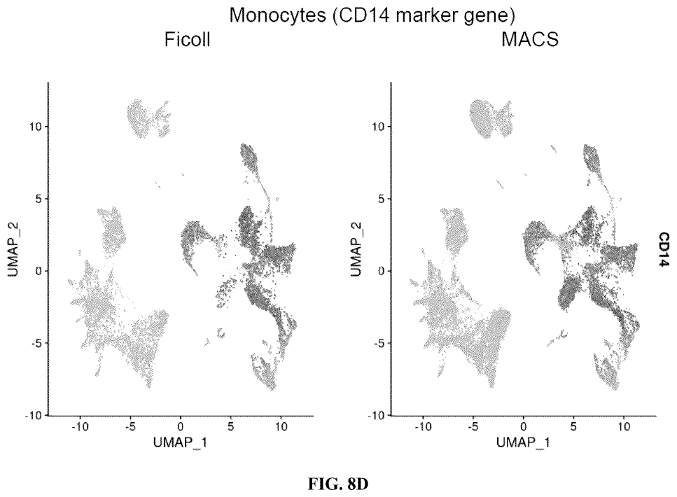

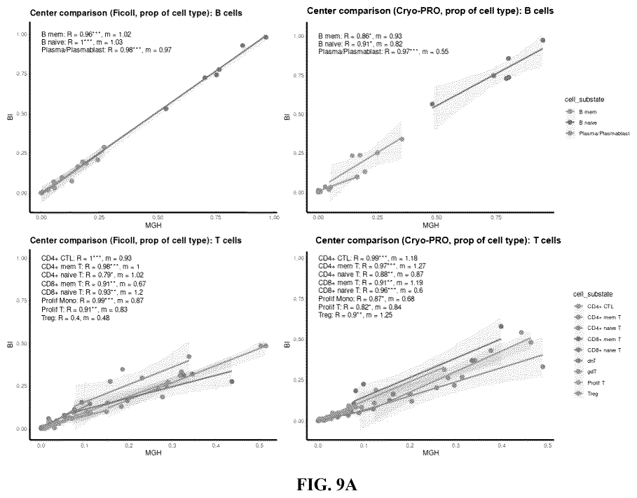

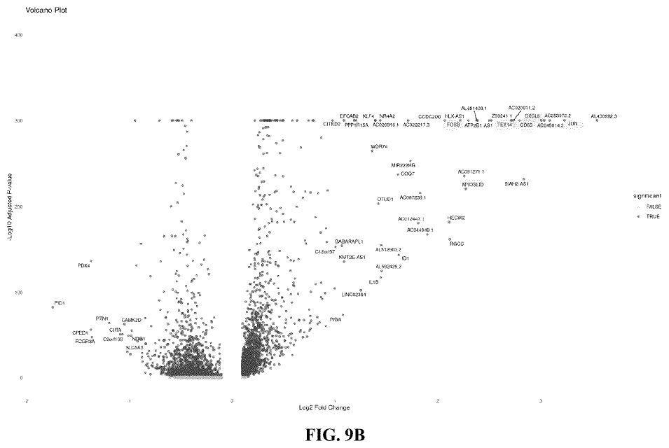

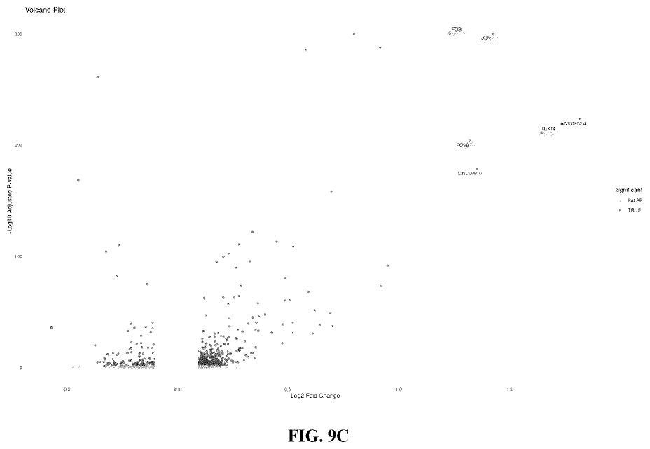

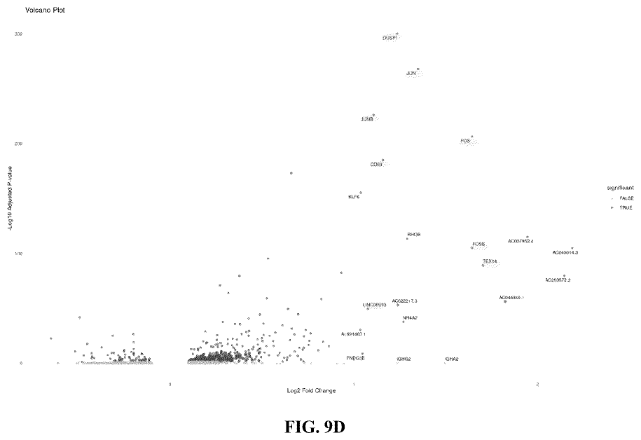

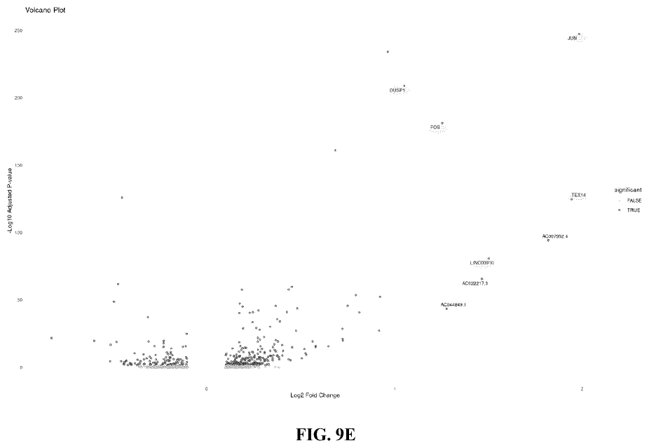

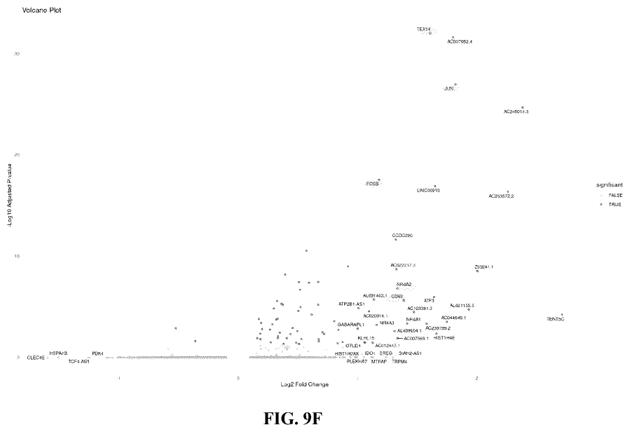

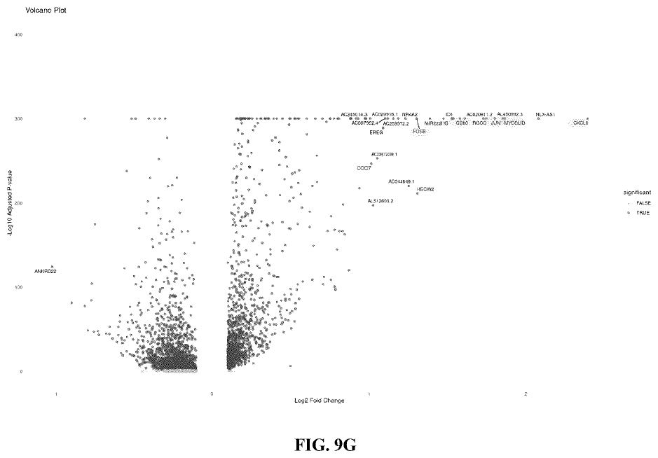

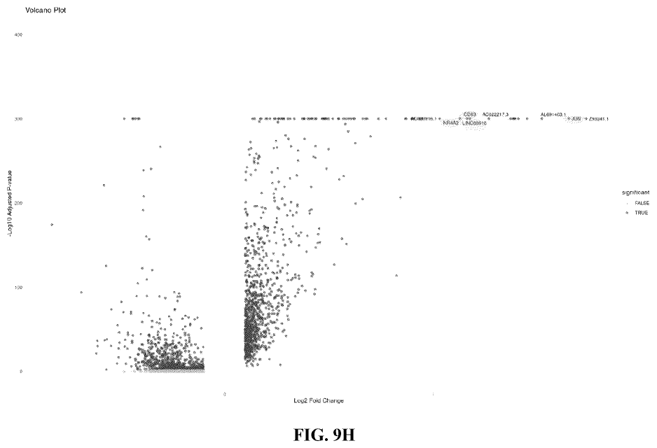

Embodiments of the present disclosure are shown in FIGS. 8A-D and FIGS. 9A-H and in the following examples. FIG. 8A shows two scatter plots comparing the effectiveness of Ficoll and MACS (Magnetic-Activated Cell Sorting; later incorporated into Cryo-PRO) methods for separating T cells marked by CD3 gene expression. The plots use Uniform Manifold Approximation and Projection (UMAP). FIG. 8B shows two scatter plots comparing the effectiveness of Ficoll and MACS (Magnetic-Activated Cell Sorting; later incorporated into Cryo-PRO) methods for separating B cells marked by the CD79A gene expression. The plots use Uniform Manifold Approximation and Projection (UMAP). FIG. 8C shows two scatter plots comparing the effectiveness of Ficoll and MACS (Magnetic-Activated Cell Sorting; later incorporated into Cryo-PRO) methods for separating NK cells marked by the GNLY gene expression. The plots use Uniform Manifold Approximation and Projection (UMAP). FIG. 8D shows two scatter plots comparing the effectiveness of Ficoll and MACS (Magnetic-Activated Cell Sorting; later incorporated into Cryo-PRO) methods for separating monocytes marked by the CD14 gene expression. The plots use Uniform Manifold Approximation and Projection (UMAP). FIG. 9A shows a scatter plot of proportions of transcriptional substates of B- and T-lymphocytes for technical replicates of samples processed at each of two different clinical sites, showing comparably high correlations with Cryo-PRO (right panels) compared with standard Ficoll (left panels). FIG. 9B shows presents a volcano plot illustrating the distribution of gene expression data specific to MS1 cells. FIG. 9C shows a volcano plot illustrating the distribution of gene expression data specific to T cells. FIG. 9D shows a volcano plot illustrating the distribution of gene expression data specific to B cells. FIG. 9E shows a volcano plot illustrating the distribution of gene expression data specific to Natural killer cells. FIG. 9F shows a volcano plot illustrating the distribution of gene expression data specific to dendritic cells. FIG. 9G shows a volcano plot illustrating the distribution of gene expression data specific to monocytes. FIG. 9H shows a volcano plot illustrating the distribution of gene expression data for all cells.

Those skilled in the art will recognize, or be able to ascertain using no more than routine experimentation, many equivalents to the specific embodiments of the disclosure described herein. Such equivalents are intended to be encompassed by the following claims.

### Example 2: Sample Collection, Storage, and Processing Strategies

Patients greater than 18 years of age who presented to the Emergency Departments (EDs) with clinical concern for sepsis or septic shock with associated organ dysfunction were enrolled in the study. Up to 10 mL of blood was obtained from patients and processing was initiated onsite using two methods: 1) standard Ficoll gradient separation from whole blood by following standard procedures for isolating and freezing PBMCs, followed by −80° C. freezing; and 2) Cryo-PRO, by adding 10% dimethyl sulfoxide (DMSO) to a final volume of 10% in 1 mL aliquots of fresh whole blood and immediately freezing at −80° C. To enable comparison of processing outcomes by site, for a subset of patients, up to 20 mL of blood (separated in two 10-mL tubes) was obtained; one tube was immediately couriered to the other clinical site while one tube remained at the enrolling site. Processing using both Cryo-PRO and Ficoll began at the same time upon sample receipt at the receiving site. Blood from one healthy donor was obtained and processed using both Cryo-PRO and Ficoll methods. All samples were sent for long term storage at −140° C. and sequencing.

23 subjects with varying degrees of sepsis severity were selected for additional sample processing and sequencing. Septic shock requiring vasopressors was present in 15 subjects, sepsis without shock in 6 subjects, and bacterial infection not meeting Sepsis-3 criteria in 2 subjects. Bacteremia was present in 7 of the 23 subjects. The median patient age was 66 years (IQR 62.5-76.5), with 35% women. The healthy donor was a 63 year old man.

Patient-paired frozen Cryo-PRO and Ficoll samples were processed for scRNA-seq. Processing included a magnetic red blood cell depletion step (Cryo-PRO samples only), fluorescence-activated cell sorting to recover DAPI− CD45+ CD235a− CD15− cells, and a standard workflow for droplet-based single-cell RNA capture with surface proteome measurement (10× Genomics Chromium Next GEM 5′ V2 Kit with cellular indexing of transcriptional epitope sequencing (CITE-seq)) [see Example 1: Methods]. Sample hashing was used to enable pooling of eight samples per processing batch, and to facilitate post-sequencing demultiplexing and multiplet detection. An overview of the sample collection, storage, and processing strategies is summarized in FIG. 1.

### Example 3: Cryo-PRO Yields High Quality scRNA-Seq Data with Minimal On-Site Processing Time

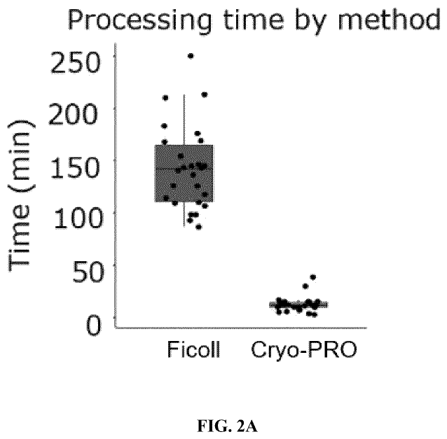

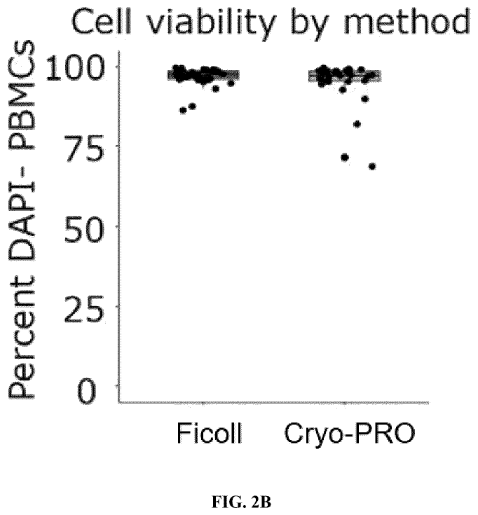

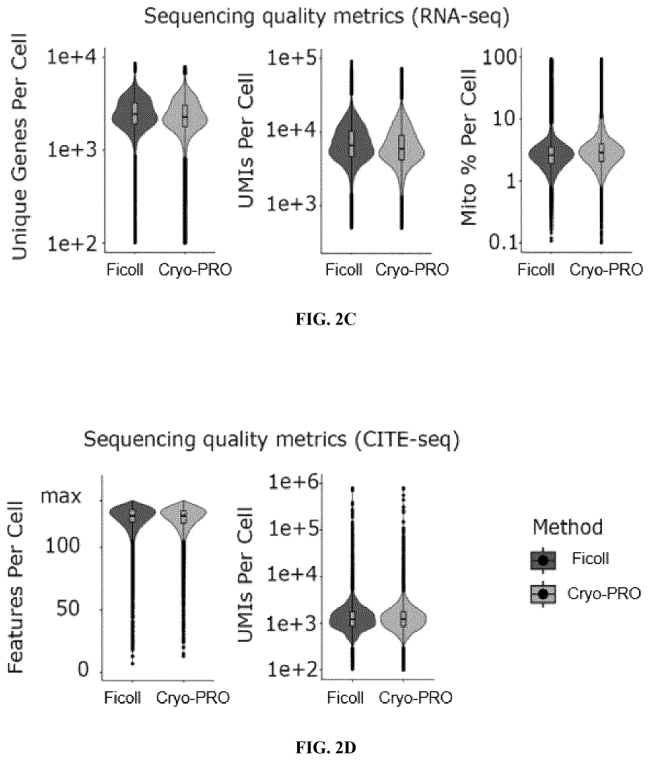

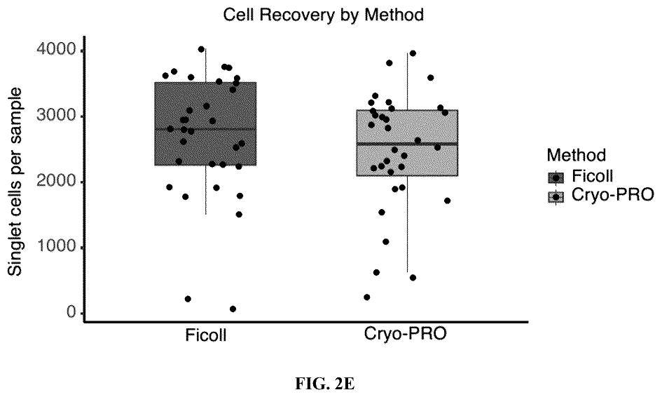

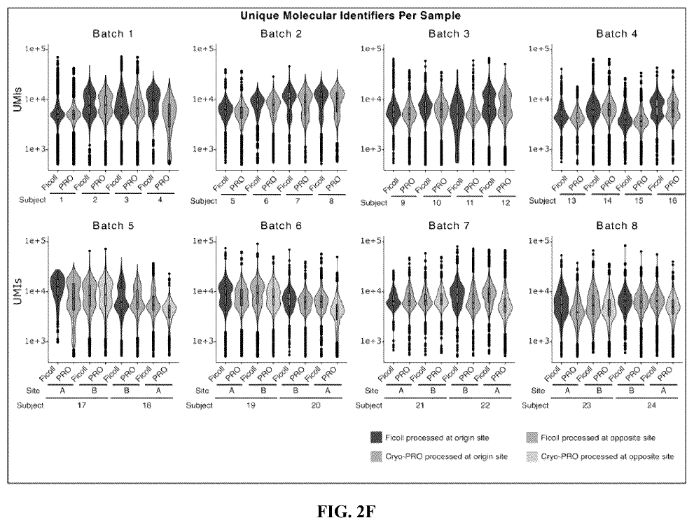

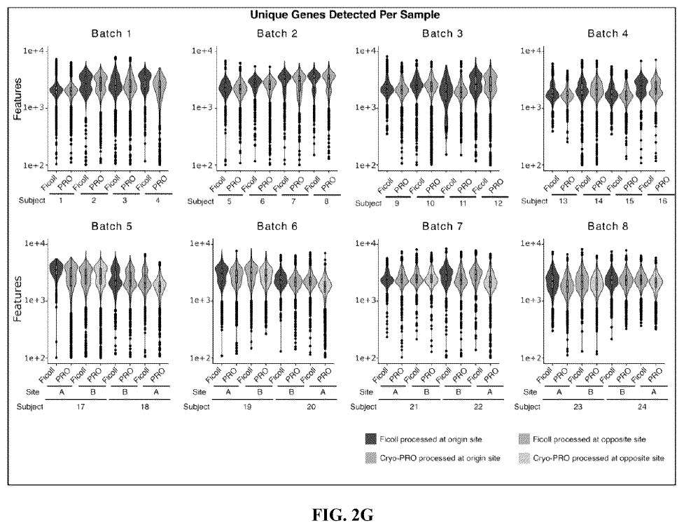

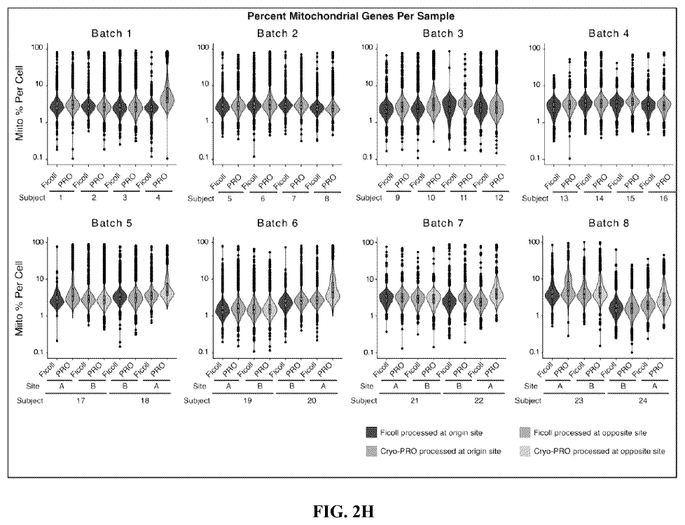

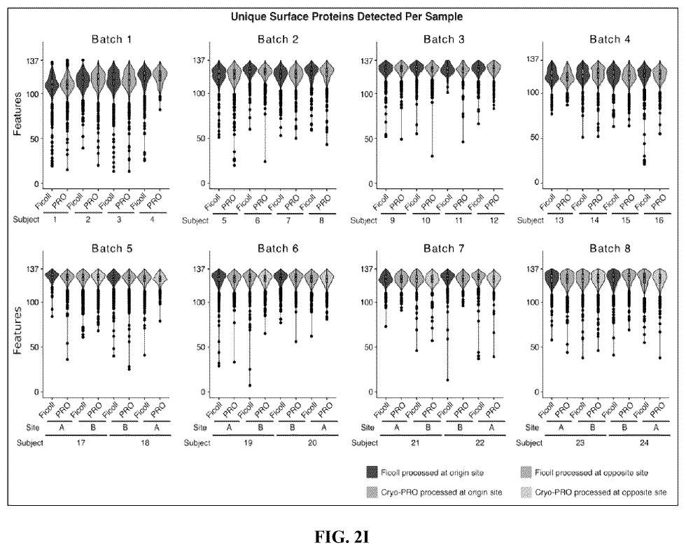

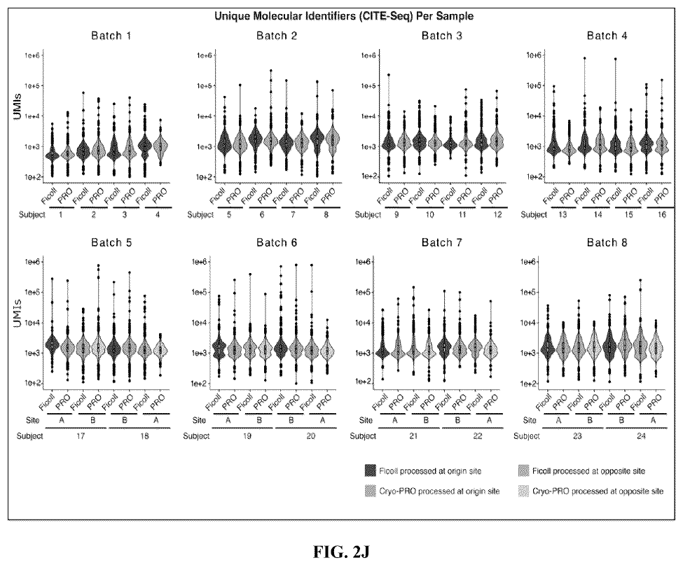

The mean time required for complete on-site processing (from blood draw to storage at −80° C.) for Ficoll samples was 2 hours and 23 minutes (SD: 40 minutes), while Cryo-PRO samples required an average of 13 minutes (SD: 7 minutes) (FIG. 2A). The proportion of viable PBMCs recovered with either Cryo-PRO or onsite Ficoll separation was estimated using live/dead staining during flow cytometry sorting. The mean proportion of live (DAPI−) CD235a− CD15− CD45+ cells for Ficoll samples was 96.7% (SD 3.0%) and 94.1% (SD 8.4%) for Cryo-PRO samples (FIG. 2B).

The Cellranger pipeline (10× Genomics) was used to process the raw sequencing data, and the Seurat V5 package in R was used for subsequent analysis of single-cell sequencing data (Example 1: Methods). Multiplets (cells associated with more than one patient hashtag) were removed from analysis. An average of 2,690 (SD 950) and 2,472 (SD 918) singlet cells per sample were recovered for the Ficoll and Cryo-PRO methods respectively (FIG. 2D). Sequencing quality was assessed using standard metrics: 1) number of genes sequenced per cell, 2) number of unique molecular identifiers (UMIs) per cell, and 3) percent of mitochondrial genes sequenced per cell (FIG. 2C). Higher numbers of genes per cell and UMIs per cell indicate greater per-cell transcript recovery, while a greater percentage of mitochondrial genes suggests cell damage. Quality metrics showed similar distributions between methods (FIG. 2D). The majority of cells (97.6% from Ficoll processing and 94.4% from Cryo-PRO) passed commonly-used quality thresholds (i.e., >250 genes per cell, >1,000 UMIs, and <10% mitochondrial genes). Detection of antibody-derived tags (ADT) used for CITE-seq surface proteome measurement was similar between the two methods (FIG. 2E). Quality metrics were similar between methods at an individual patient level (FIG. 2F, FIG. 2G, FIG. 2H, FIG. 2I, and FIG. 2J).

### Example 4: Cryo-PRO Enables Identification of Immune Cell Transcriptional Substates and Gene Expression Patterns

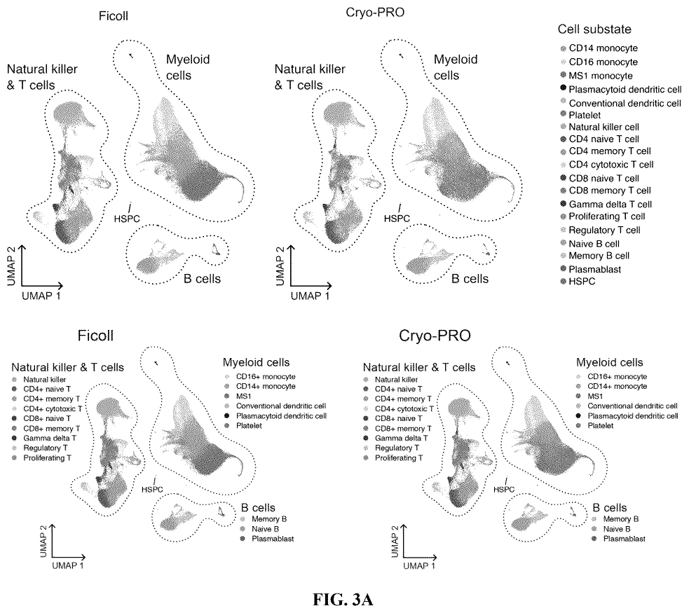

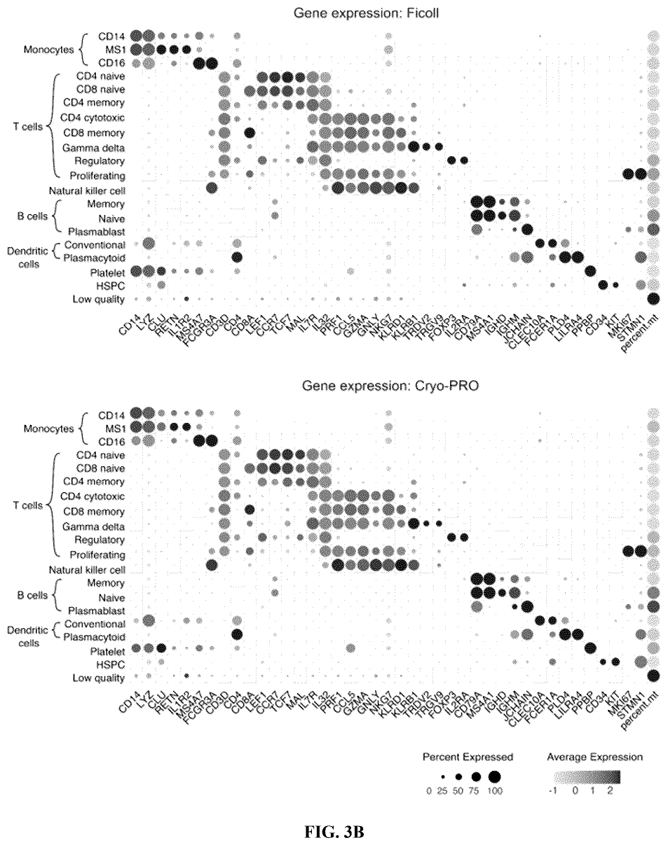

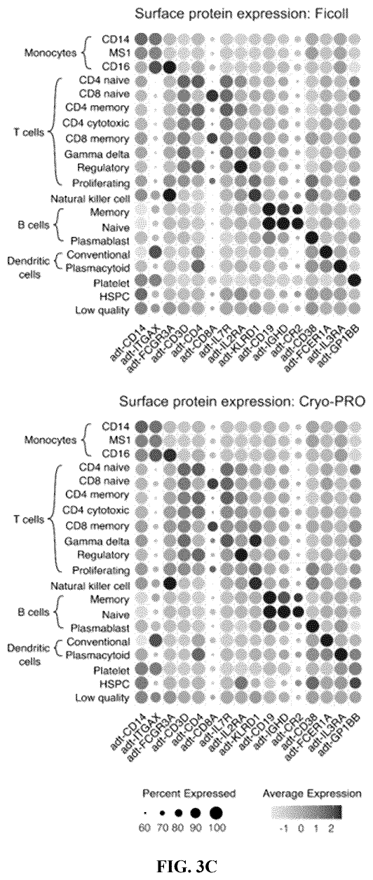

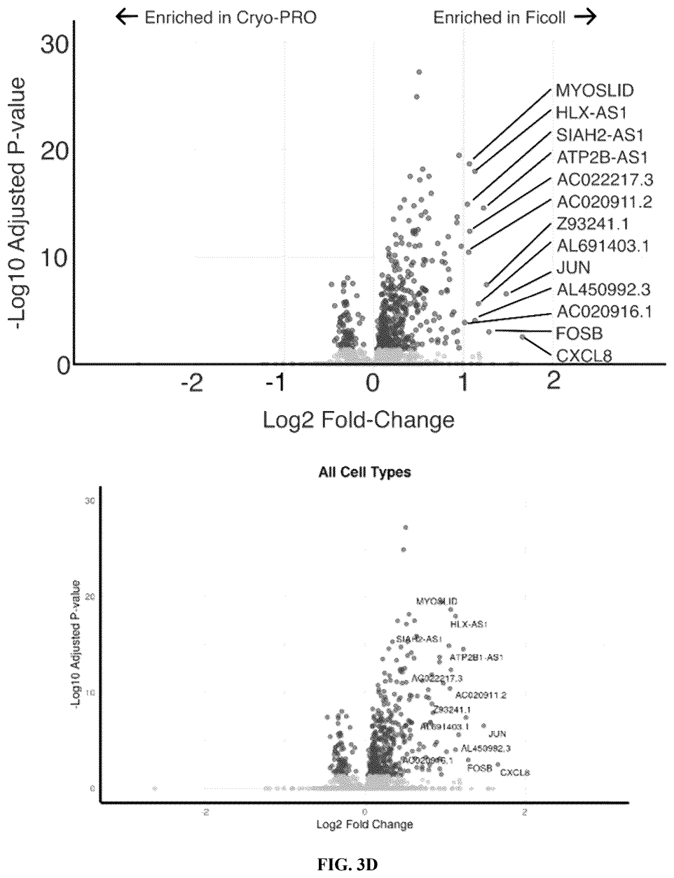

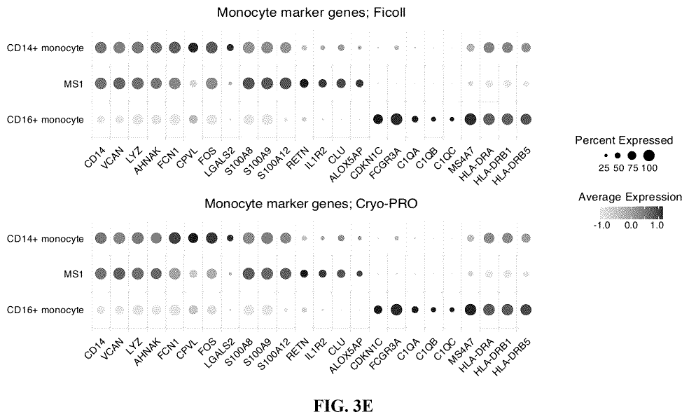

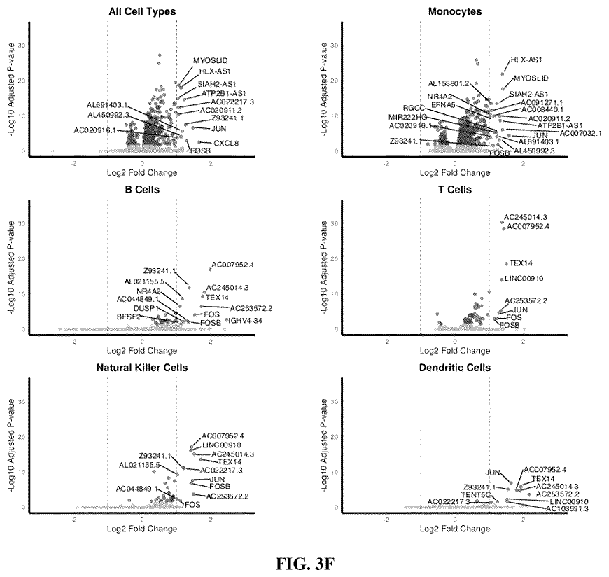

Next whether Cryo-PRO generates scRNA-seq datasets of sufficient quality to reproduce biologically relevant results compared to Ficoll was assessed. ScRNA-seq analysis was performed separately for cells obtained from each processing method (86,083 cells for Ficoll and 79,089 cells for Cryo-PRO) to ensure independent identification of cell identity and gene expression patterns (see Example 1: Methods). Clusters of dead and dying cells, indicated by the dominance of mitochondrial genes, were removed from further analysis as an extended quality control measure. Transcriptionally similar cells that expressed canonical marker genes for the major mononuclear immune cell lineages (i.e., T cells, B cells, natural killer cells, monocytes, and dendritic cells) were identified. Subclustering within each cell type identified higher-resolution clusters of cells with additional transcriptional similarity (i.e., cell substates, e.g., CD4+ memory T cells, naive B cells, etc.), which were classified by comparison with reference datasets. All the major mononuclear immune cell lineages, divided into a total of 17 cell substates, were identified from cells isolated using either Ficoll or Cryo-PRO (FIG. 3A). The analysis demonstrated enrichment of the novel MS1 monocyte state as previously found in cohorts of patients with sepsis. The average expression of key identifying genes (FIG. 3B, color scale) and cell surface proteins (FIG. 3C, color scale) were similar between Cryo-PRO and Ficoll methods for each cell type and substate, as were the proportion of cells for which these features could be detected (FIG. 3B and FIG. 3C, dot size).

Top marker genes to distinguish each cell substate were identified using the FindMarkers function in Seurat; rank was determined by fold-change of the gene expression within cells of each cluster compared to the cells outside of the cluster. Of the top 30 marker genes for each cell type, shared genes between processing methods ranged from 24 to 28, and shared genes between processing methods for cell substates from ranged from 14 to 29 (see e.g., Tables 1-31). Table 1 shows the top 30 marker genes that were identified by cell type. Table 2 shows identified marker genes within the top 30 marker genes identified in Table 1 that are shared between the Ficoll and Cryo-PRO methods. Notably, a high degree of overlap of top MS1 marker genes between processing methods was observed, with 21 of the top 30 marker genes in common (Table 1) and similar expression patterns of key MS1 marker genes (FIG. 3E). Table 3 shows the top 30 identified marker genes by cell substate.

TABLE 1
Top 30 identified marker genes by cell type

| | | Ficoll | | Cryo-PRO | |
|---|---|---|---|---|---|
| | cluster | gene | avg_log2FC | gene | avg_log2FC |
| 1 | B.cell | VPREB3 | 9.05654425 | VPREB3 | 9.00964513 |
| 2 | B.cell | IGHV5-78 | 8.85628772 | IGHD | 8.85341976 |
| 3 | B.cell | SLC38A11 | 8.74915974 | IGHV5-78 | 8.70697223 |
| 4 | B.cell | IGHD | 8.70441175 | LINC02397 | 8.70395467 |
| 5 | B.cell | CD24 | 8.66810585 | CD19 | 8.69623257 |
| 6 | B.cell | CD79A | 8.58116794 | CD79A | 8.68613453 |
| 7 | B.cell | LINC02397 | 8.57834137 | COL19A1 | 8.63351581 |
| 8 | B.cell | PAX5 | 8.48762063 | MS4A1 | 8.54325801 |
| 9 | B.cell | CD19 | 8.46212958 | PAX5 | 8.48782372 |
| 10 | B.cell | FCRL1 | 8.42970205 | CD24 | 8.48592383 |
| 11 | B.cell | COL19A1 | 8.37409263 | FCRL1 | 8.46851804 |
| 12 | B.cell | MS4A1 | 8.37359897 | SLC38A11 | 8.44221058 |
| 13 | B.cell | FCRL2 | 8.24135978 | FCRLA | 8.39563982 |
| 14 | B.cell | FCRLA | 8.21631342 | TCL1A | 8.31380068 |
| 15 | B.cell | LINC00926 | 8.19984811 | LINC00926 | 8.30186566 |
| 16 | B.cell | FCRL5 | 8.14496277 | LINC01857 | 8.20236731 |
| 17 | B.cell | TCL1A | 8.11898086 | FCRL5 | 8.14813136 |
| 18 | B.cell | LINC01857 | 8.07780442 | FCRL2 | 8.10199888 |
| 19 | B.cell | IGHM | 7.8386025 | IGHM | 7.97699951 |
| 20 | B.cell | EBF1 | 7.58601912 | CD22 | 7.94518551 |
| 21 | B.cell | BLK | 7.48723438 | FAM30A | 7.83810519 |
| 22 | B.cell | BACE2 | 7.47501695 | EBF1 | 7.6196058 |
| 23 | B.cell | BANK1 | 7.45013253 | BLK | 7.46739234 |
| 24 | B.cell | FCER2 | 7.43449738 | POU2AF1 | 7.44726626 |
| 25 | B.cell | FAM30A | 7.4025994 | BANK1 | 7.38141164 |
| 26 | B.cell | CD200 | 7.30834434 | CD200 | 7.38093586 |
| 27 | B.cell | POU2AF1 | 7.20872843 | FCER2 | 7.21919652 |
| 28 | B.cell | TNFRSF13C | 7.15483429 | NIBAN3 | 7.19883504 |
| 29 | B.cell | NIBAN3 | 7.00356347 | PCDH9 | 7.16525615 |
| 30 | B.cell | IGKC | 6.92101543 | TNFRSF13C | 7.16108313 |
| 31 | DC | LRRC26 | 12.3573505 | LRRC26 | 12.6790817 |
| 32 | DC | SCT | 10.9849677 | SCT | 12.1504625 |
| 33 | DC | SHD | 10.712115 | SHD | 11.1362381 |
| 34 | DC | CLEC4C | 9.81413619 | LINC01478 | 9.94773338 |
| 35 | DC | LINC01478 | 9.31248565 | CLEC4C | 9.50847721 |
| 36 | DC | FCER1A | 8.73991641 | FCER1A | 9.07782291 |
| 37 | DC | P3H2 | 8.53954266 | P3H2 | 8.42403025 |
| 38 | DC | PTCRA | 8.05734449 | CUX2 | 8.29691209 |
| 39 | DC | LILRA4 | 7.64871193 | PTPRS | 7.78784334 |
| 40 | DC | CUX2 | 7.5919236 | MAP1A | 7.69241105 |
| 41 | DC | DNASE1L3 | 7.55564341 | DNASE1L3 | 7.61870905 |
| 42 | DC | PLD4 | 7.38993825 | PLD4 | 7.41151485 |
| 43 | DC | PTPRS | 7.35765555 | LILRA4 | 7.39900321 |
| 44 | DC | TIFAB | 7.20221789 | FAM160A1 | 7.35988251 |
| 45 | DC | MAP1A | 7.10345061 | LAMP5 | 7.35322167 |
| 46 | DC | PPM1J | 7.10115918 | SERPINF1 | 7.21748748 |
| 47 | DC | LAMP5 | 7.09089284 | PPM1J | 7.16970443 |
| 48 | DC | TPM2 | 7.06531685 | TIFAB | 7.09999124 |
| 49 | DC | SERPINF1 | 7.02560305 | LINC01374 | 7.02704914 |
| 50 | DC | AC023590.1 | 6.94410848 | TPM2 | 6.98440841 |
| 51 | DC | FAM160A1 | 6.91434392 | AC023590.1 | 6.87804988 |
| 52 | DC | LINC01374 | 6.75635925 | PACSIN1 | 6.87484632 |
| 53 | DC | SMPD3 | 6.67415707 | SCAMP5 | 6.82618616 |
| 54 | DC | ENHO | 6.56142689 | RASD1 | 6.75918046 |
| 55 | DC | PACSIN1 | 6.16668572 | SMPD3 | 6.72765008 |
| 56 | DC | TLR9 | 6.13747381 | ENHO | 6.45251274 |
| 57 | DC | SLC35F3 | 5.94286597 | SMIM5 | 6.45204454 |
| 58 | DC | CD1C | 5.94203078 | PTCRA | 6.35458208 |
| 59 | DC | AC007381.1 | 5.92193273 | SLC35F3 | 6.21232424 |
| 60 | DC | EPHB1 | 5.91206623 | TNFRSF21 | 6.126032 |
| 61 | Monocyte | S100A12 | 5.81428751 | S100A12 | 5.83765626 |
| 62 | Monocyte | S100A9 | 5.68564651 | S100A9 | 5.80467518 |
| 63 | Monocyte | RBP7 | 5.58714209 | S100A8 | 5.68131747 |
| 64 | Monocyte | S100A8 | 5.57969433 | RNASE2 | 5.45003641 |
| 65 | Monocyte | FOLR3 | 5.42963683 | RBP7 | 5.40090325 |
| 66 | Monocyte | CSTA | 5.26158312 | CSTA | 5.28056281 |
| 67 | Monocyte | RNASE2 | 5.23305758 | AC020656.1 | 5.19519057 |
| 68 | Monocyte | SMIM25 | 5.20303778 | TMEM176A | 5.13169304 |
| 69 | Monocyte | SERPINA1 | 5.16333322 | MCEMP1 | 5.11358004 |
| 70 | Monocyte | RETN | 5.09818486 | CFD | 4.94937256 |
| 71 | Monocyte | TMEM176A | 5.09616284 | SERPINA1 | 4.91655407 |
| 72 | Monocyte | MCEMP1 | 5.06447929 | AIF1 | 4.91146549 |
| 73 | Monocyte | LILRA5 | 5.01231132 | LYZ | 4.90957598 |
| 74 | Monocyte | TMEM176B | 4.93188899 | GPBAR1 | 4.88907903 |
| 75 | Monocyte | CDA | 4.91330093 | RETN | 4.88642768 |
| 76 | Monocyte | CFD | 4.88206139 | SMIM25 | 4.75785396 |
| 77 | Monocyte | GPBAR1 | 4.86857742 | CD14 | 4.73951421 |
| 78 | Monocyte | AC020656.1 | 4.86531225 | TMEM176B | 4.73273516 |
| 79 | Monocyte | AIF1 | 4.83276911 | LILRA5 | 4.68033776 |
| 80 | Monocyte | CD14 | 4.8066708 | IGSF6 | 4.58291176 |
| 81 | Monocyte | LYZ | 4.76599325 | FPR1 | 4.53480202 |
| 82 | Monocyte | LILRA2 | 4.72519469 | LILRA2 | 4.5140799 |
| 83 | Monocyte | APOBEC3A | 4.70901885 | CD68 | 4.50845957 |
| 84 | Monocyte | CD68 | 4.67227123 | ASGR1 | 4.47591299 |
| 85 | Monocyte | MGST1 | 4.67150546 | CLEC4E | 4.44356254 |
| 86 | Monocyte | LST1 | 4.67083031 | FCGR1A | 4.41947813 |
| 87 | Monocyte | ASGR1 | 4.65109397 | KCNE3 | 4.39745921 |
| 88 | Monocyte | FCN1 | 4.64377159 | IFI30 | 4.39434506 |
| 89 | Monocyte | KCNE3 | 4.63474122 | MNDA | 4.38400076 |
| 90 | Monocyte | CYP1B1 | 4.60588925 | LST1 | 4.3724768 |
| 91 | Natural.killer | KIR2DL4 | 5.95045844 | KIR2DL1 | 6.29050743 |
| 92 | Natural.killer | SH2D1B | 5.67551286 | KIR2DL4 | 6.24109988 |
| 93 | Natural.killer | KIR2DL1 | 5.63919211 | SH2D1B | 5.95696661 |
| 94 | Natural.killer | PTGDS | 5.2349928 | AKR1C3 | 5.64347734 |
| 95 | Natural.killer | KLRC1 | 5.21664688 | PTGDS | 5.33307574 |
| 96 | Natural.killer | AKR1C3 | 5.14600675 | KLRC1 | 5.26767884 |
| 97 | Natural.killer | KLRF1 | 5.00847038 | LAIR2 | 5.20204687 |
| 98 | Natural.killer | LAIR2 | 4.90746731 | KLRF1 | 5.19826043 |
| 99 | Natural.killer | MYOM2 | 4.83414037 | KIR3DL1 | 5.17029081 |
| 100 | Natural.killer | SPON2 | 4.7803021 | MYOM2 | 4.99828076 |
| 101 | Natural.killer | TRDC | 4.76825586 | GNLY | 4.9401634 |
| 102 | Natural.killer | KIR3DL1 | 4.71870504 | SPON2 | 4.93070832 |
| 103 | Natural.killer | GNLY | 4.64830409 | TRDC | 4.86200192 |
| 104 | Natural.killer | CLIC3 | 4.49445519 | NMUR1 | 4.78192069 |
| 105 | Natural.killer | NMUR1 | 4.49135873 | CLIC3 | 4.69673565 |
| 106 | Natural.killer | NCR1 | 4.48611671 | CCL3 | 4.69400749 |
| 107 | Natural.killer | CD160 | 4.42751622 | NCR1 | 4.68317354 |
| 108 | Natural.killer | TMIGD2 | 4.39229504 | GZMB | 4.64404799 |
| 109 | Natural.killer | GZMB | 4.36211652 | TMIGD2 | 4.56837804 |
| 110 | Natural.killer | XCL2 | 4.30112352 | PRF1 | 4.48865534 |
| 111 | Natural.killer | PRF1 | 4.24671485 | FGFBP2 | 4.34885225 |
| 112 | Natural.killer | LINC00299 | 4.17639965 | AREG | 4.31862975 |
| 113 | Natural.killer | TNFRSF18 | 4.09745433 | XCL2 | 4.29718709 |
| 114 | Natural.killer | CCL3 | 4.08670153 | S1PR5 | 4.19308789 |
| 115 | Natural.killer | IL2RB | 4.07641295 | LINC00299 | 4.16317948 |
| 116 | Natural.killer | FGFBP2 | 4.05329347 | PRSS23 | 4.14968818 |
| 117 | Natural.killer | LINGO2 | 4.05131947 | CCL4 | 4.11068715 |
| 118 | Natural.killer | S1PR5 | 4.02306966 | IL2RB | 4.08860473 |
| 119 | Natural.killer | PRSS23 | 3.9638524 | KLRD1 | 4.06790364 |
| 120 | Natural.killer | IL18RAP | 3.93398762 | TRGC1 | 4.05272461 |
| 121 | T.cell | CD8B | 5.65827148 | CD8B | 5.79194249 |
| 122 | T.cell | CD3D | 5.56722998 | CD3D | 5.69742735 |
| 123 | T.cell | MAL | 5.16477386 | MAL | 5.53060284 |
| 124 | T.cell | CD3G | 5.06541796 | CD3G | 5.2355697 |
| 125 | T.cell | CD5 | 5.02590208 | SIRPG | 4.98267868 |
| 126 | T.cell | UBASH3A | 4.78433306 | CD5 | 4.95455008 |
| 127 | T.cell | IL7R | 4.65561787 | IL7R | 4.85556823 |
| 128 | T.cell | TRAT1 | 4.65100504 | AQP3 | 4.80489188 |
| 129 | T.cell | SIRPG | 4.64906272 | TRAT1 | 4.66676555 |
| 130 | T.cell | CD3E | 4.46882673 | CD27 | 4.54856709 |
| 131 | T.cell | AQP3 | 4.41247555 | CD3E | 4.50561819 |
| 132 | T.cell | TCF7 | 4.3934397 | TCF7 | 4.43269841 |
| 133 | T.cell | ICOS | 4.28267737 | ICOS | 4.38387486 |
| 134 | T.cell | CD27 | 4.25747529 | CD28 | 4.20273198 |
| 135 | T.cell | CD8A | 4.11076666 | CD8A | 4.19909707 |
| 136 | T.cell | SIT1 | 4.08042786 | LINC01550 | 4.16790537 |
| 137 | T.cell | CD28 | 4.04972558 | TRAC | 4.07246467 |
| 138 | T.cell | TRAC | 4.04040225 | SIT1 | 3.92270139 |
| 139 | T.cell | IL32 | 3.85369356 | IL32 | 3.87939882 |
| 140 | T.cell | GPR171 | 3.80450097 | GPR171 | 3.78230173 |
| 141 | T.cell | CD6 | 3.61797906 | LEF1 | 3.7266249 |
| 142 | T.cell | CISH | 3.56614048 | CAMK4 | 3.47560256 |
| 143 | T.cell | LEF1 | 3.53693647 | CD6 | 3.45175722 |
| 144 | T.cell | NPDC1 | 3.45838287 | NPDC1 | 3.38776184 |
| 145 | T.cell | THEMIS | 3.41339452 | PRKCQ-AS1 | 3.37733581 |
| 146 | T.cell | TRABD2A | 3.40397924 | CISH | 3.33494335 |
| 147 | T.cell | PRKCQ-AS1 | 3.39988695 | THEMIS | 3.31185319 |
| 148 | T.cell | CAMK4 | 3.38842141 | INPP4B | 3.22588981 |
| 149 | T.cell | INPP4B | 3.22323971 | RGCC | 3.18866049 |
| 150 | T.cell | KCNA3 | 3.16119683 | KCNA3 | 3.17569623 |

TABLE 2
Marker genes identified in the top 30 that are
shared between the Ficoll and Cryo-PRO methods

| | | | Ficoll | Cryo-PRO |
|---|---|---|---|---|
| | cluster | gene | avg_log2FC | avg_log2FC |
| 1 | B.cell | BANK1 | 7.45013253 | 7.38141164 |
| 2 | B.cell | BLK | 7.48723438 | 7.46739234 |
| 3 | B.cell | CD19 | 8.46212958 | 8.69623257 |
| 4 | B.cell | CD200 | 7.30834434 | 7.38093586 |
| 5 | B.cell | CD24 | 8.66810585 | 8.48592383 |
| 6 | B.cell | CD79A | 8.58116794 | 8.68613453 |
| 7 | B.cell | COL19A1 | 8.37409263 | 8.63351581 |
| 8 | B.cell | EBF1 | 7.58601912 | 7.6196058 |
| 9 | B.cell | FAM30A | 7.4025994 | 7.83810519 |
| 10 | B.cell | FCER2 | 7.43449738 | 7.21919652 |
| 11 | B.cell | FCRL1 | 8.42970205 | 8.46851804 |
| 12 | B.cell | FCRL2 | 8.24135978 | 8.10199888 |
| 13 | B.cell | FCRL5 | 8.14496277 | 8.14813136 |
| 14 | B.cell | FCRLA | 8.21631342 | 8.39563982 |
| 15 | B.cell | IGHD | 8.70441175 | 8.85341976 |
| 16 | B.cell | IGHM | 7.8386025 | 7.97699951 |
| 17 | B.cell | IGHV5-78 | 8.85628772 | 8.70697223 |
| 18 | B.cell | LINC00926 | 8.19984811 | 8.30186566 |
| 19 | B.cell | LINC01857 | 8.07780442 | 8.20236731 |
| 20 | B.cell | LINC02397 | 8.57834137 | 8.70395467 |
| 21 | B.cell | MS4A1 | 8.37359897 | 8.54325801 |
| 22 | B.cell | NIBAN3 | 7.00356347 | 7.19883504 |
| 23 | B.cell | PAX5 | 8.48762063 | 8.48782372 |
| 24 | B.cell | POU2AF1 | 7.20872843 | 7.44726626 |
| 25 | B.cell | SLC38A11 | 8.74915974 | 8.44221058 |
| 26 | B.cell | TCL1A | 8.11898086 | 8.31380068 |
| 27 | B.cell | TNFRSF13C | 7.15483429 | 7.16108313 |
| 28 | B.cell | VPREB3 | 9.05654425 | 9.00964513 |
| 29 | DC | AC023590.1 | 6.94410848 | 6.87804988 |
| 30 | DC | CLEC4C | 9.81413619 | 9.50847721 |
| 31 | DC | CUX2 | 7.5919236 | 8.29691209 |
| 32 | DC | DNASE1L3 | 7.55564341 | 7.61870905 |
| 33 | DC | ENHO | 6.56142689 | 6.45251274 |
| 34 | DC | FAM160A1 | 6.91434392 | 7.35988251 |
| 35 | DC | FCER1A | 8.73991641 | 9.07782291 |
| 36 | DC | LAMP5 | 7.09089284 | 7.35322167 |
| 37 | DC | LILRA4 | 7.64871193 | 7.39900321 |
| 38 | DC | LINC01374 | 6.75635925 | 7.02704914 |
| 39 | DC | LINC01478 | 9.31248565 | 9.94773338 |
| 40 | DC | LRRC26 | 12.3573505 | 12.6790817 |
| 41 | DC | MAP1A | 7.10345061 | 7.69241105 |
| 42 | DC | P3H2 | 8.53954266 | 8.42403025 |
| 43 | DC | PACSIN1 | 6.16668572 | 6.87484632 |
| 44 | DC | PLD4 | 7.38993825 | 7.41151485 |
| 45 | DC | PPM1J | 7.10115918 | 7.16970443 |
| 46 | DC | PTCRA | 8.05734449 | 6.35458208 |
| 47 | DC | PTPRS | 7.35765555 | 7.78784334 |
| 48 | DC | SCT | 10.9849677 | 12.1504625 |
| 49 | DC | SERPINF1 | 7.02560305 | 7.21748748 |
| 50 | DC | SHD | 10.712115 | 11.1362381 |
| 51 | DC | SLC35F3 | 5.94286597 | 6.21232424 |
| 52 | DC | SMPD3 | 6.67415707 | 6.72765008 |
| 53 | DC | TIFAB | 7.20221789 | 7.09999124 |
| 54 | DC | TPM2 | 7.06531685 | 6.98440841 |
| 55 | Monocyte | AC020656.1 | 4.86531225 | 5.19519057 |
| 56 | Monocyte | AIF1 | 4.83276911 | 4.91146549 |
| 57 | Monocyte | ASGR1 | 4.65109397 | 4.47591299 |
| 58 | Monocyte | CD14 | 4.8066708 | 4.73951421 |
| 59 | Monocyte | CD68 | 4.67227123 | 4.50845957 |
| 60 | Monocyte | CFD | 4.88206139 | 4.94937256 |
| 61 | Monocyte | CSTA | 5.26158312 | 5.28056281 |
| 62 | Monocyte | GPBAR1 | 4.86857742 | 4.88907903 |
| 63 | Monocyte | KCNE3 | 4.63474122 | 4.39745921 |
| 64 | Monocyte | LILRA2 | 4.72519469 | 4.5140799 |
| 65 | Monocyte | LILRA5 | 5.01231132 | 4.68033776 |
| 66 | Monocyte | LST1 | 4.67083031 | 4.3724768 |
| 67 | Monocyte | LYZ | 4.76599325 | 4.90957598 |
| 68 | Monocyte | MCEMP1 | 5.06447929 | 5.11358004 |
| 69 | Monocyte | RBP7 | 5.58714209 | 5.40090325 |
| 70 | Monocyte | RETN | 5.09818486 | 4.88642768 |
| 71 | Monocyte | RNASE2 | 5.23305758 | 5.45003641 |
| 72 | Monocyte | S100A12 | 5.81428751 | 5.83765626 |
| 73 | Monocyte | S100A8 | 5.57969433 | 5.68131747 |
| 74 | Monocyte | S100A9 | 5.68564651 | 5.80467518 |
| 75 | Monocyte | SERPINA1 | 5.16333322 | 4.91655407 |
| 76 | Monocyte | SMIM25 | 5.20303778 | 4.75785396 |
| 77 | Monocyte | TMEM176A | 5.09616284 | 5.13169304 |
| 78 | Monocyte | TMEM176B | 4.93188899 | 4.73273516 |
| 79 | Natural.killer | AKR1C3 | 5.14600675 | 5.64347734 |
| 80 | Natural.killer | CCL3 | 4.08670153 | 4.69400749 |
| 81 | Natural.killer | CLIC3 | 4.49445519 | 4.69673565 |
| 82 | Natural.killer | FGFBP2 | 4.05329347 | 4.34885225 |
| 83 | Natural.killer | GNLY | 4.64830409 | 4.9401634 |
| 84 | Natural.killer | GZMB | 4.36211652 | 4.64404799 |
| 85 | Natural.killer | IL2RB | 4.07641295 | 4.08860473 |
| 86 | Natural.killer | KIR2DL1 | 5.63919211 | 6.29050743 |
| 87 | Natural.killer | KIR2DL4 | 5.95045844 | 6.24109988 |
| 88 | Natural.killer | KIR3DL1 | 4.71870504 | 5.17029081 |
| 89 | Natural.killer | KLRC1 | 5.21664688 | 5.26767884 |
| 90 | Natural.killer | KLRF1 | 5.00847038 | 5.19826043 |
| 91 | Natural.killer | LAIR2 | 4.90746731 | 5.20204687 |
| 92 | Natural.killer | LINC00299 | 4.17639965 | 4.16317948 |
| 93 | Natural.killer | MYOM2 | 4.83414037 | 4.99828076 |
| 94 | Natural.killer | NCR1 | 4.48611671 | 4.68317354 |
| 95 | Natural.killer | NMUR1 | 4.49135873 | 4.78192069 |
| 96 | Natural.killer | PRF1 | 4.24671485 | 4.48865534 |
| 97 | Natural.killer | PRSS23 | 3.9638524 | 4.14968818 |
| 98 | Natural.killer | PTGDS | 5.2349928 | 5.33307574 |
| 99 | Natural.killer | S1PR5 | 4.02306966 | 4.19308789 |
| 100 | Natural.killer | SH2D1B | 5.67551286 | 5.95696661 |
| 101 | Natural.killer | SPON2 | 4.7803021 | 4.93070832 |
| 102 | Natural.killer | TMIGD2 | 4.39229504 | 4.56837804 |
| 103 | Natural.killer | TRDC | 4.76825586 | 4.86200192 |
| 104 | Natural.killer | XCL2 | 4.30112352 | 4.29718709 |
| 105 | T.cell | AQP3 | 4.41247555 | 4.80489188 |
| 106 | T.cell | CAMK4 | 3.38842141 | 3.47560256 |
| 107 | T.cell | CD27 | 4.25747529 | 4.54856709 |
| 108 | T.cell | CD28 | 4.04972558 | 4.20273198 |
| 109 | T.cell | CD3D | 5.56722998 | 5.69742735 |
| 110 | T.cell | CD3E | 4.46882673 | 4.50561819 |
| 111 | T.cell | CD3G | 5.06541796 | 5.2355697 |
| 112 | T.cell | CD5 | 5.02590208 | 4.95455008 |
| 113 | T.cell | CD6 | 3.61797906 | 3.45175722 |
| 114 | T.cell | CD8A | 4.11076666 | 4.19909707 |
| 115 | T.cell | CD8B | 5.65827148 | 5.79194249 |
| 116 | T.cell | CISH | 3.56614048 | 3.33494335 |
| 117 | T.cell | GPR171 | 3.80450097 | 3.78230173 |
| 118 | T.cell | ICOS | 4.28267737 | 4.38387486 |
| 119 | T.cell | IL32 | 3.85369356 | 3.87939882 |
| 120 | T.cell | IL7R | 4.65561787 | 4.85556823 |
| 121 | T.cell | INPP4B | 3.22323971 | 3.22588981 |
| 122 | T.cell | KCNA3 | 3.16119683 | 3.17569623 |
| 123 | T.cell | LEF1 | 3.53693647 | 3.7266249 |
| 124 | T.cell | MAL | 5.16477386 | 5.53060284 |
| 125 | T.cell | NPDC1 | 3.45838287 | 3.38776184 |
| 126 | T.cell | PRKCQ-AS1 | 3.39988695 | 3.37733581 |
| 127 | T.cell | SIRPG | 4.64906272 | 4.98267868 |
| 128 | T.cell | SIT1 | 4.08042786 | 3.92270139 |
| 129 | T.cell | TCF7 | 4.3934397 | 4.43269841 |
| 130 | T.cell | THEMIS | 3.41339452 | 3.31185319 |
| 131 | T.cell | TRAC | 4.04040225 | 4.07246467 |
| 132 | T.cell | TRAT1 | 4.65100504 | 4.66676555 |

TABLE 3
Top 30 identified marker genes by cell substate

| | | Ficoll | | Cryo-PRO | |
|---|---|---|---|---|---|
| | cluster | gene | avg_log2FC | gene | avg_log2FC |
| 1 | CD14+ | LGALS2 | 3.18037115 | LGALS2 | 3.14874797 |
| | monocyte | | | | |
| 2 | CD14+ | PID1 | 2.86510028 | EGR1 | 2.59407847 |
| | monocyte | | | | |
| 3 | CD14+ | IL1B | 2.26477007 | TEX14 | 2.51102233 |
| | monocyte | | | | |
| 4 | CD14+ | F13A1 | 2.19275264 | AC007952.4 | 2.4837257 |
| | monocyte | | | | |
| 5 | CD14+ | NRG1 | 2.15461483 | FOS | 2.16800196 |
| | monocyte | | | | |
| 6 | CD14+ | EGR1 | 2.13758555 | FOSB | 2.01555335 |
| | monocyte | | | | |
| 7 | CD14+ | CYP27A1 | 2.0991734 | CYP27A1 | 1.93704474 |
| | monocyte | | | | |
| 8 | CD14+ | MARCO | 2.09841037 | IL1RN | 1.92774899 |
| | monocyte | | | | |
| 9 | CD14+ | ARHGEF10L | 2.07317554 | RBP7 | 1.92153464 |
| | monocyte | | | | |
| 10 | CD14+ | SH3PXD2B | 2.06036221 | CLEC4A | 1.90585587 |
| | monocyte | | | | |
| 11 | CD14+ | TGFBI | 2.03960118 | CLEC4E | 1.83798982 |
| | monocyte | | | | |
| 12 | CD14+ | RTN1 | 2.03320372 | MARCKS | 1.8186204 |
| | monocyte | | | | |
| 13 | CD14+ | CPVL | 2.00372671 | FCGR1A | 1.80171051 |
| | monocyte | | | | |
| 14 | CD14+ | MARCKS | 1.91719867 | CSTA | 1.78603106 |
| | monocyte | | | | |
| 15 | CD14+ | RAB13 | 1.87804547 | RAB32 | 1.77364844 |
| | monocyte | | | | |
| 16 | CD14+ | APOBEC3A | 1.87783566 | TGFBI | 1.77017143 |
| | monocyte | | | | |
| 17 | CD14+ | FCGR1A | 1.87277436 | MS4A6A | 1.75059954 |
| | monocyte | | | | |
| 18 | CD14+ | CPM | 1.86745808 | CD14 | 1.74970303 |
| | monocyte | | | | |
| 19 | CD14+ | CLEC4A | 1.86149583 | TREM1 | 1.74877132 |
| | monocyte | | | | |
| 20 | CD14+ | TCN2 | 1.85703477 | TMEM176A | 1.74363066 |
| | monocyte | | | | |
| 21 | CD14+ | DUSP6 | 1.85135868 | SGK1 | 1.7300354 |
| | monocyte | | | | |
| 22 | CD14+ | CLEC4E | 1.84684168 | AC005280.2 | 1.72752977 |
| | monocyte | | | | |
| 23 | CD14+ | ZNF385A | 1.81449427 | CPVL | 1.71355794 |
| | monocyte | | | | |
| 24 | CD14+ | DOCK4 | 1.79328242 | APOBEC3A | 1.70711287 |
| | monocyte | | | | |
| 25 | CD14+ | HPSE | 1.78037089 | LYZ | 1.70191546 |
| | monocyte | | | | |
| 26 | CD14+ | AC005280.2 | 1.77350023 | DUSP6 | 1.68766582 |
| | monocyte | | | | |
| 27 | CD14+ | MAP3K7CL | 1.76455897 | IGSF6 | 1.68609704 |
| | monocyte | | | | |
| 28 | CD14+ | MAFB | 1.76072838 | ASGR1 | 1.68208957 |
| | monocyte | | | | |
| 29 | CD14+ | PDK4 | 1.76049874 | FCGR2A | 1.68190547 |
| | monocyte | | | | |
| 30 | CD14+ | FCGR2A | 1.75387278 | ZNF385A | 1.65754145 |
| | monocyte | | | | |
| 31 | CD16+ | CDKN1C | 6.49920063 | CDKN1C | 6.35097567 |
| | monocyte | | | | |
| 32 | CD16+ | AC020651.2 | 6.41792976 | C1QC | 6.30613768 |
| | monocyte | | | | |
| 33 | CD16+ | C1QC | 6.27389325 | C1QB | 6.06260482 |
| | monocyte | | | | |
| 34 | CD16+ | C1QB | 5.98661419 | C1QA | 5.8540532 |
| | monocyte | | | | |
| 35 | CD16+ | C1QA | 5.78452678 | AC020651.2 | 5.57092415 |
| | monocyte | | | | |
| 36 | CD16+ | CKB | 5.38951096 | HES4 | 4.93132789 |
| | monocyte | | | | |
| 37 | CD16+ | HES4 | 5.13769924 | ZNF703 | 4.72684493 |
| | monocyte | | | | |
| 38 | CD16+ | ZNF703 | 4.53003531 | NR4A1 | 4.07735707 |
| | monocyte | | | | |
| 39 | CD16+ | FMNL2 | 4.52330573 | FCGR3B | 4.02017349 |
| | monocyte | | | | |
| 40 | CD16+ | FCGR3B | 4.43779393 | CEACAM3 | 3.97284324 |
| | monocyte | | | | |
| 41 | CD16+ | NEURL1 | 4.34747023 | NEURL1 | 3.94962401 |
| | monocyte | | | | |
| 42 | CD16+ | CEACAM3 | 4.10626279 | FMNL2 | 3.82474303 |
| | monocyte | | | | |
| 43 | CD16+ | PPM1N | 3.98297641 | BATF3 | 3.71211291 |
| | monocyte | | | | |
| 44 | CD16+ | CASP5 | 3.83426195 | PPM1N | 3.57568029 |
| | monocyte | | | | |
| 45 | CD16+ | BATF3 | 3.72998478 | CASP5 | 3.55290917 |
| | monocyte | | | | |
| 46 | CD16+ | MS4A7 | 3.54490551 | MS4A7 | 3.37815039 |
| | monocyte | | | | |
| 47 | CD16+ | NR4A1 | 3.50738711 | CTSL | 3.25608138 |
| | monocyte | | | | |
| 48 | CD16+ | ICAM4 | 3.29749113 | RHOB | 3.24840823 |
| | monocyte | | | | |
| 49 | CD16+ | TPPP3 | 3.296837 | SMIM25 | 3.24712181 |
| | monocyte | | | | |
| 50 | CD16+ | EBI3 | 3.28006813 | EBI3 | 3.21079395 |
| | monocyte | | | | |
| 51 | CD16+ | CTSL | 3.20724164 | TPPP3 | 3.20804798 |
| | monocyte | | | | |
| 52 | CD16+ | TNFRSF8 | 3.18481975 | GPBAR1 | 3.09988987 |
| | monocyte | | | | |
| 53 | CD16+ | SMIM25 | 3.17917813 | FCGR3A | 2.94471835 |
| | monocyte | | | | |
| 54 | CD16+ | FCGR3A | 3.07119148 | MRAS | 2.89820826 |
| | monocyte | | | | |
| 55 | CD16+ | MRAS | 3.05030897 | LST1 | 2.86684367 |
| | monocyte | | | | |
| 56 | CD16+ | MSR1 | 3.04899641 | MSR1 | 2.84851498 |
| | monocyte | | | | |
| 57 | CD16+ | RHOB | 3.02391404 | ZDHHC1 | 2.81128564 |
| | monocyte | | | | |
| 58 | CD16+ | GPBAR1 | 2.95514675 | TNFRSF8 | 2.80001987 |
| | monocyte | | | | |
| 59 | CD16+ | MGLL | 2.94208503 | LILRB1 | 2.76076846 |
| | monocyte | | | | |
| 60 | CD16+ | LILRB1 | 2.94090647 | WARS | 2.73786645 |
| | monocyte | | | | |
| 61 | CD4+ cytotoxic T | ZNF683 | 4.99845371 | LINC00892 | 4.15226279 |
| 62 | CD4+ cytotoxic T | LINC00892 | 3.80999035 | CD40LG | 3.49382368 |
| 63 | CD4+ cytotoxic T | GZMH | 3.15412116 | GZMH | 3.21192508 |
| 64 | CD4+ cytotoxic T | CD40LG | 3.01382825 | TMEM273 | 2.90244075 |
| 65 | CD4+ cytotoxic T | CD320 | 2.72460312 | CD320 | 2.78874917 |
| 66 | CD4+ cytotoxic T | CD5 | 2.68737734 | CD5 | 2.78086797 |
| 67 | CD4+ cytotoxic T | KLRG1 | 2.62441025 | CD6 | 2.68354124 |
| 68 | CD4+ cytotoxic T | CD6 | 2.60442207 | KLRG1 | 2.61141758 |
| 69 | CD4+ cytotoxic T | LINC01871 | 2.52468354 | LINC01871 | 2.56404717 |
| 70 | CD4+ cytotoxic T | CD3D | 2.47335317 | CD3G | 2.54290891 |
| 71 | CD4+ cytotoxic T | CD3G | 2.44503906 | SLAMF1 | 2.53768836 |
| 72 | CD4+ cytotoxic T | MYBL1 | 2.43917844 | CD3D | 2.4657633 |
| 73 | CD4+ cytotoxic T | SLAMF1 | 2.36914304 | THEMIS | 2.40073771 |
| 74 | CD4+ cytotoxic T | IL32 | 2.31094419 | CD2 | 2.36148424 |
| 75 | CD4+ cytotoxic T | THEMIS | 2.30573346 | IL32 | 2.35039742 |
| 76 | CD4+ cytotoxic T | CD2 | 2.29184702 | ITM2A | 2.30333356 |
| 77 | CD4+ cytotoxic T | FGFBP2 | 2.26935732 | SIT1 | 2.26905696 |
| 78 | CD4+ cytotoxic T | CCL5 | 2.24576714 | CCL5 | 2.2368662 |
| 79 | CD4+ cytotoxic T | SIT1 | 2.225823 | MYBL1 | 2.22313164 |
| 80 | CD4+ cytotoxic T | C12orf75 | 2.19923977 | GZMA | 2.22278352 |
| 81 | CD4+ cytotoxic T | CXCR3 | 2.15317717 | CD3E | 2.20437984 |
| 82 | CD4+ cytotoxic T | CD3E | 2.13899251 | C12orf75 | 2.18797545 |
| 83 | CD4+ cytotoxic T | AC006369.1 | 2.13651161 | FGFBP2 | 2.17869073 |
| 84 | CD4+ cytotoxic T | MXRA7 | 2.13508207 | TRG-AS1 | 2.08278195 |
| 85 | CD4+ cytotoxic T | FCRL6 | 2.08429345 | TGFBR3 | 2.07339076 |
| 86 | CD4+ cytotoxic T | ITM2A | 2.06867793 | S1PR1 | 1.97799629 |
| 87 | CD4+ cytotoxic T | TGFBR3 | 2.01911486 | AC006369.1 | 1.9237077 |
| 88 | CD4+ cytotoxic T | GZMA | 2.01397905 | GZMM | 1.91728591 |
| 89 | CD4+ cytotoxic T | S1PR1 | 1.94908848 | SAMD3 | 1.90362257 |
| 90 | CD4+ cytotoxic T | PPP2R2B | 1.94597623 | LCK | 1.86468562 |
| 91 | CD4+ memory T | CD40LG | 3.77752706 | AQP3 | 3.76934533 |
| 92 | CD4+ memory T | AQP3 | 3.65383905 | CD40LG | 3.60859233 |
| 93 | CD4+ memory T | IL7R | 3.36867005 | IL7R | 3.3710388 |
| 94 | CD4+ memory T | TNFRSF4 | 3.31590729 | MAL | 3.24385998 |
| 95 | CD4+ memory T | CD28 | 3.18821362 | TNFRSF4 | 3.20356282 |
| 96 | CD4+ memory T | LINC02273 | 3.18393344 | CD28 | 3.19173661 |
| 97 | CD4+ memory T | TRAT1 | 3.17900533 | LINC02273 | 3.12039975 |
| 98 | CD4+ memory T | MAL | 3.14255345 | TRAT1 | 3.10985568 |
| 99 | CD4+ memory T | FAAH2 | 3.10725456 | NPDC1 | 3.08993035 |
| 100 | CD4+ memory T | ICOS | 3.07025527 | FAAH2 | 2.95396402 |
| 101 | CD4+ memory T | NPDC1 | 3.0269522 | TNFRSF25 | 2.91672487 |
| 102 | CD4+ memory T | TNFRSF25 | 2.97813189 | ICOS | 2.90303834 |
| 103 | CD4+ memory T | AC139720.1 | 2.95993771 | AC139720.1 | 2.8565984 |
| 104 | CD4+ memory T | GPR171 | 2.95184058 | GPR171 | 2.83214767 |
| 105 | CD4+ memory T | PASK | 2.90115117 | TCF7 | 2.71422123 |
| 106 | CD4+ memory T | DPP4 | 2.80194368 | INPP4B | 2.71240532 |
| 107 | CD4+ memory T | LTB | 2.73400901 | LTB | 2.6882496 |
| 108 | CD4+ memory T | INPP4B | 2.71861498 | SIRPG | 2.64656761 |
| 109 | CD4+ memory T | LSR | 2.66473043 | CD5 | 2.56609395 |
| 110 | CD4+ memory T | CD5 | 2.64667015 | TESPA1 | 2.5603322 |
| 111 | CD4+ memory T | TCF7 | 2.60216081 | RGCC | 2.46818004 |
| 112 | CD4+ memory T | GATA3 | 2.5310852 | CISH | 2.46491025 |
| 113 | CD4+ memory T | SIRPG | 2.52377869 | CMTM8 | 2.42537957 |
| 114 | CD4+ memory T | ANK3 | 2.52235236 | GATA3 | 2.41395433 |
| 115 | CD4+ memory T | CISH | 2.51253535 | SUSD3 | 2.39441716 |
| 116 | CD4+ memory T | TESPA1 | 2.49792094 | RCAN3 | 2.3854696 |
| 117 | CD4+ memory T | CMTM8 | 2.48842466 | GPR183 | 2.36061459 |
| 118 | CD4+ memory T | AP3M2 | 2.40315736 | UBASH3A | 2.35785509 |
| 119 | CD4+ memory T | SUSD3 | 2.39154411 | CAMK4 | 2.34568899 |
| 120 | CD4+ memory T | UBASH3A | 2.37654491 | FAM102A | 2.33233367 |
| 121 | CD4+ naive T | ADTRP | 5.00930261 | ADTRP | 4.9158537 |
| 122 | CD4+ naive T | ANKRD55 | 4.04602565 | ANKRD55 | 4.20376131 |
| 123 | CD4+ naive T | CHRM3-AS2 | 3.92496248 | CHRM3-AS2 | 3.84878456 |
| 124 | CD4+ naive T | EDA | 3.80859599 | EDA | 3.78047821 |
| 125 | CD4+ naive T | TSHZ2 | 3.68912033 | TSHZ2 | 3.74243283 |
| 126 | CD4+ naive T | CCR7 | 3.61273776 | CCR7 | 3.65068775 |
| 127 | CD4+ naive T | MAL | 3.48810319 | MAL | 3.54466191 |
| 128 | CD4+ naive T | TCF7 | 3.42070515 | TCF7 | 3.46889752 |
| 129 | CD4+ naive T | EPHX2 | 3.41617569 | EPHX2 | 3.45146263 |
| 130 | CD4+ naive T | AC139720.1 | 3.33252444 | AC139720.1 | 3.39814918 |
| 131 | CD4+ naive T | TRABD2A | 3.24500693 | LEF1 | 3.30553608 |
| 132 | CD4+ naive T | BEX3 | 3.21283058 | TRABD2A | 3.27345324 |
| 133 | CD4+ naive T | LEF1 | 3.20548621 | LINC01550 | 3.23436107 |
| 134 | CD4+ naive T | LINC01550 | 3.19528302 | BEX3 | 3.06900075 |
| 135 | CD4+ naive T | PRKCQ-AS1 | 2.95765027 | PRKCQ-AS1 | 2.88669061 |
| 136 | CD4+ naive T | ITGA6 | 2.88294633 | ITGA6 | 2.87736022 |
| 137 | CD4+ naive T | LDLRAP1 | 2.86669594 | LDLRAP1 | 2.8586837 |
| 138 | CD4+ naive T | RNF157 | 2.78313328 | RNF157 | 2.84271279 |
| 139 | CD4+ naive T | CD27 | 2.64518073 | DPP4 | 2.7063506 |
| 140 | CD4+ naive T | RASGRF2 | 2.56150502 | TRAT1 | 2.66975345 |
| 141 | CD4+ naive T | TRAT1 | 2.50433609 | CD27 | 2.61213168 |
| 142 | CD4+ naive T | FHIT | 2.45563823 | CMTM8 | 2.61069895 |
| 143 | CD4+ naive T | FAAH2 | 2.44602107 | FAAH2 | 2.57013015 |
| 144 | CD4+ naive T | CMTM8 | 2.41501474 | RGCC | 2.56282098 |
| 145 | CD4+ naive T | TMEM204 | 2.40672906 | TMEM204 | 2.50330836 |
| 146 | CD4+ naive T | RCAN3 | 2.39664421 | CD40LG | 2.44909921 |
| 147 | CD4+ naive T | MYC | 2.382581 | RCAN3 | 2.39996383 |
| 148 | CD4+ naive T | RETREG1 | 2.37466395 | OXNAD1 | 2.39812887 |
| 149 | CD4+ naive T | CAMK4 | 2.36229414 | BEX2 | 2.35898777 |
| 150 | CD4+ naive T | OXNAD1 | 2.3575801 | SUSD3 | 2.35811099 |
| 151 | CD8+ memory T | AC243829.2 | 4.7457815 | GZMK | 4.88149912 |
| 152 | CD8+ memory T | CD8A | 4.60869113 | CD8A | 4.81576709 |
| 153 | CD8+ memory T | CD8B | 4.51398688 | CD8B | 4.79740042 |
| 154 | CD8+ memory T | LAG3 | 4.46985097 | LAG3 | 4.61272097 |
| 155 | CD8+ memory T | GZMK | 4.28949503 | LINC02446 | 4.18633964 |
| 156 | CD8+ memory T | LINC02446 | 3.82011496 | TRGC2 | 3.69637637 |
| 157 | CD8+ memory T | TRGC2 | 3.68622728 | KLRC4 | 3.54416819 |
| 158 | CD8+ memory T | KLRC4 | 3.60552028 | GZMH | 3.33116384 |
| 159 | CD8+ memory T | GZMH | 3.17266634 | CCL5 | 3.22010744 |
| 160 | CD8+ memory T | CCL5 | 3.10011729 | EOMES | 2.99025687 |
| 161 | CD8+ memory T | EOMES | 2.94105532 | KLRG1 | 2.9688218 |
| 162 | CD8+ memory T | KLRG1 | 2.9354101 | CD3D | 2.91072147 |
| 163 | CD8+ memory T | TIGIT | 2.81556264 | CD3G | 2.89890959 |
| 164 | CD8+ memory T | FCRL6 | 2.72459902 | LINC01871 | 2.85344762 |
| 165 | CD8+ memory T | SH2D1A | 2.65601391 | SH2D1A | 2.66052443 |
| 166 | CD8+ memory T | CD3G | 2.63126041 | AC006369.1 | 2.56934422 |
| 167 | CD8+ memory T | CD3D | 2.61546513 | TIGIT | 2.56003122 |
| 16 | CD8+ memory T | CCL4L2 | 2.58898834 | FCRL6 | 2.55134318 |
| 169 | CD8+ memory T | LINC01871 | 2.5509141 | THEMIS | 2.50174269 |
| 170 | CD8+ memory T | KLRK1 | 2.52875605 | CD2 | 2.50030936 |
| 171 | CD8+ memory T | DUSP2 | 2.46793893 | KLRK1 | 2.49175836 |
| 172 | CD8+ memory T | AC006369.1 | 2.46777648 | IL32 | 2.47536559 |
| 173 | CD8+ memory T | F2R | 2.45346053 | CD3E | 2.47222834 |
| 174 | CD8+ memory T | GZMA | 2.41617621 | DUSP2 | 2.43991913 |
| 175 | CD8+ memory T | GZMM | 2.33014658 | GZMA | 2.41762761 |
| 176 | CD8+ memory T | C12orf75 | 2.31338858 | GZMM | 2.39276784 |
| 177 | CD8+ memory T | THEMIS | 2.31198233 | C12orf75 | 2.35337794 |
| 178 | CD8+ memory T | IL32 | 2.31069511 | CCL4L2 | 2.34757403 |
| 179 | CD8+ memory T | CD2 | 2.30626052 | F2R | 2.32652107 |
| 180 | CD8+ memory T | CD3E | 2.27756026 | SIT1 | 2.30054407 |
| 181 | CD8+ naive T | LINC02446 | 4.46100205 | CD248 | 7.45408436 |
| 182 | CD8+ naive T | NELL2 | 4.10911509 | LINC02446 | 4.53075842 |
| 183 | CD8+ naive T | S100B | 3.65144731 | NELL2 | 4.08525863 |
| 184 | CD8+ naive T | CD8B | 3.60983035 | CD8B | 3.64346413 |
| 185 | CD8+ naive T | NT5E | 3.41989427 | S100B | 3.43280894 |
| 186 | CD8+ naive T | CCR7 | 2.94641117 | LEF1-AS1 | 3.3194254 |
| 187 | CD8+ naive T | TCF7 | 2.69939685 | CCR7 | 3.17888221 |
| 188 | CD8+ naive T | LEF1 | 2.69783107 | CHRM3-AS2 | 2.8444214 |
| 189 | CD8+ naive T | CD27 | 2.67734197 | LEF1 | 2.83672768 |
| 190 | CD8+ naive T | LDLRAP1 | 2.61673587 | TCF7 | 2.8226261 |
| 191 | CD8+ naive T | TRABD2A | 2.5815034 | TRABD2A | 2.8082527 |
| 192 | CD8+ naive T | LINC01550 | 2.46494726 | CD27 | 2.73025665 |
| 193 | CD8+ naive T | SIRPG | 2.4519693 | PRKCQ-AS1 | 2.60376255 |
| 194 | CD8+ naive T | RASGRF2 | 2.43359148 | LDLRAP1 | 2.59430233 |
| 195 | CD8+ naive T | OXNAD1 | 2.41670825 | MAL | 2.56653888 |
| 196 | CD8+ naive T | CD8A | 2.37695848 | BEX3 | 2.48275144 |
| 197 | CD8+ naive T | LSR | 2.34854638 | RNF157 | 2.4674866 |
| 198 | CD8+ naive T | PRKCQ-AS1 | 2.33458012 | SIRPG | 2.42860594 |
| 199 | CD8+ naive T | PASK | 2.31371999 | PASK | 2.41287197 |
| 200 | CD8+ naive T | MAL | 2.30669643 | RASGRF2 | 2.40576177 |
| 201 | CD8+ naive T | FBXO32 | 2.29302233 | OXNAD1 | 2.38139379 |
| 202 | CD8+ naive T | RNF157 | 2.23250259 | LSR | 2.35185297 |
| 203 | CD8+ naive T | CISH | 2.12569731 | CD8A | 2.33197115 |
| 204 | CD8+ naive T | CAMK4 | 2.08464308 | LINC01550 | 2.31690883 |
| 205 | CD8+ naive T | RETREG1 | 2.08179808 | TMEM204 | 2.21941117 |
| 206 | CD8+ naive T | BEX3 | 2.07334353 | FBXO32 | 2.20847125 |
| 207 | CD8+ naive T | NOSIP | 2.06861326 | NOSIP | 2.141436 |
| 208 | CD8+ naive T | TMEM204 | 2.06032561 | RETREG1 | 2.08672117 |
| 209 | CD8+ naive T | NPDC1 | 2.02072798 | APBA2 | 2.06486942 |
| 210 | CD8+ naive T | IL7R | 2.01272633 | LDHB | 2.00744918 |
| 211 | Conventional | FCER1A | 8.6011106 | FCER1A | 9.00423101 |
| | dendritic cell | | | | |
| 212 | Conventional | CD1E | 8.54125177 | CD1E | 8.32114488 |
| | dendritic cell | | | | |
| 213 | Conventional | ENHO | 7.270269 | ENHO | 7.20709625 |
| | dendritic cell | | | | |
| 214 | Conventional | CD1C | 6.65087289 | CD1C | 6.41045827 |
| | dendritic cell | | | | |
| 215 | Conventional | ST18 | 5.95146225 | PKIB | 5.99764325 |
| | dendritic cell | | | | |
| 216 | Conventional | PKIB | 5.67321577 | ST18 | 5.53407193 |
| | dendritic cell | | | | |
| 217 | Conventional | CLEC10A | 5.38644785 | CLEC10A | 5.36778799 |
| | dendritic cell | | | | |
| 218 | Conventional | PPP1R14A | 5.24053004 | SLC41A2 | 5.14659954 |
| | dendritic cell | | | | |
| 219 | Conventional | SLC41A2 | 5.1419215 | MRC1 | 4.854756 |
| | dendritic cell | | | | |
| 220 | Conventional | CLIC2 | 5.06015779 | NDRG2 | 4.77531857 |
| | dendritic cell | | | | |
| 221 | Conventional | MRC1 | 4.9012207 | DEPTOR | 4.75881978 |
| | dendritic cell | | | | |
| 222 | Conventional | HLA-DQA1 | 4.89849929 | CLIC2 | 4.73537643 |
| | dendritic cell | | | | |
| 223 | Conventional | GHRL | 4.69228975 | PPP1R14A | 4.68195171 |
| | dendritic cell | | | | |
| 224 | Conventional | NDRG2 | 4.62053701 | HLA-DQA1 | 4.59059362 |
| | dendritic cell | | | | |
| 225 | Conventional | C19orf33 | 4.55898573 | GHRL | 4.56999007 |
| | dendritic cell | | | | |
| 226 | Conventional | DEPTOR | 4.5504998 | P2RY6 | 4.45253099 |
| | dendritic cell | | | | |
| 227 | Conventional | SH3BP4 | 4.53941938 | SERPINF2 | 4.37541721 |
| | dendritic cell | | | | |
| 228 | Conventional | P2RY6 | 4.46605843 | ZBTB46 | 4.36196483 |
| | dendritic cell | | | | |
| 229 | Conventional | ZBTB46 | 4.37865151 | SH3BP4 | 4.30212709 |
| | dendritic cell | | | | |
| 230 | Conventional | SERPINF2 | 4.37395333 | CYP2S1 | 4.22832109 |
| | dendritic cell | | | | |
| 231 | Conventional | CYP2S1 | 4.28678943 | ATP1B1 | 4.07051857 |
| | dendritic cell | | | | |
| 232 | Conventional | HLA-DQB1 | 4.27964344 | CCSER1 | 4.02528205 |
| | dendritic cell | | | | |
| 233 | Conventional | CRIP3 | 4.25149775 | C1orf54 | 4.02362806 |
| | dendritic cell | | | | |
| 234 | Conventional | CCSER1 | 4.24847159 | HLA-DQB1 | 4.01687909 |
| | dendritic cell | | | | |
| 235 | Conventional | LGMN | 4.11378941 | SERPINF1 | 3.92340986 |
| | dendritic cell | | | | |
| 236 | Conventional | PLD4 | 4.10747715 | LGMN | 3.83709524 |
| | dendritic cell | | | | |
| 237 | Conventional | NAPSA | 4.08343091 | NEGR1 | 3.79694607 |
| | dendritic cell | | | | |
| 238 | Conventional | HLA-DPB1 | 4.05477117 | HLA-DPB1 | 3.7926446 |
| | dendritic cell | | | | |
| 239 | Conventional | NEGR1 | 4.04269912 | NET1 | 3.75639334 |
| | dendritic cell | | | | |
| 240 | Conventional | ATP1B1 | 4.04227152 | HLA-DPA1 | 3.6944335 |
| | dendritic cell | | | | |
| 241 | Gamma delta T | TRDV2 | 9.08115853 | SLC4A10 | 8.06230344 |
| 242 | Gamma delta T | SLC4A10 | 7.10063622 | TRDV2 | 7.33163542 |
| 243 | Gamma delta T | TRGV9 | 5.36901884 | TRAV1-2 | 5.2362657 |
| 244 | Gamma delta T | CXCR6 | 4.47200618 | CXCR6 | 5.03552533 |
| 245 | Gamma delta T | GZMK | 3.63377776 | TRGV9 | 4.30686676 |
| 246 | Gamma delta T | DPP4 | 3.20956386 | GZMK | 4.18503883 |
| 247 | Gamma delta T | LAG3 | 3.16620975 | LAG3 | 3.87702092 |
| 248 | Gamma delta T | TRDC | 3.1289914 | DPP4 | 3.55553734 |
| 249 | Gamma delta T | KLRB1 | 2.99749162 | IL12RB2 | 3.28537746 |
| 250 | Gamma delta T | KLRG1 | 2.98809107 | LINC01871 | 3.22040582 |
| 251 | Gamma delta T | DUSP2 | 2.8346244 | KLRB1 | 3.20722027 |
| 252 | Gamma delta T | LINC01871 | 2.76651455 | NCR3 | 3.0016142 |
| 253 | Gamma delta T | NCR3 | 2.75932212 | IL7R | 2.97273329 |
| 254 | Gamma delta T | PBX4 | 2.71832687 | COLQ | 2.96851828 |
| 255 | Gamma delta T | IL7R | 2.64229221 | DUSP2 | 2.95315197 |
| 256 | Gamma delta T | TRGC1 | 2.62817367 | KLRG1 | 2.93133337 |
| 257 | Gamma delta T | IL18RAP | 2.53977531 | IL18RAP | 2.80672713 |
| 258 | Gamma delta T | TRAC | 2.53127127 | HPGD | 2.8023127 |
| 259 | Gamma delta T | MYBL1 | 2.46069182 | GPR171 | 2.79376594 |
| 260 | Gamma delta T | KLRC1 | 2.45096889 | PTMS | 2.74034856 |
| 261 | Gamma delta T | AC006369.1 | 2.42822178 | IL18R1 | 2.70512726 |
| 262 | Gamma delta T | HPGD | 2.39709634 | PBX4 | 2.69130579 |
| 263 | Gamma delta T | SYTL2 | 2.22632717 | IFNG-AS1 | 2.60363121 |
| 26 | Gamma delta T | MPZL3 | 2.21835809 | TRGC2 | 2.51439137 |
| 265 | Gamma delta T | GPR171 | 2.20931036 | CD69 | 2.49965161 |
| 266 | Gamma delta T | IL18R1 | 2.19573017 | TRAC | 2.46460634 |
| 267 | Gamma delta T | LYAR | 2.16409892 | KLRC1 | 2.45029498 |
| 268 | Gamma delta T | SPOCK2 | 2.15523281 | MYBL1 | 2.41588105 |
| 269 | Gamma delta T | ERN1 | 2.08358144 | PRR5 | 2.40057432 |
| 270 | Gamma delta T | PTMS | 2.06956328 | SLAMF1 | 2.35059392 |
| 271 | HSPC | AVP | 15.2912413 | CPA3 | 14.6944987 |
| 272 | HSPC | TM4SF1 | 12.9315161 | GATA2 | 13.7236984 |
| 273 | HSPC | CD34 | 11.9498564 | LINC02573 | 13.3344519 |
| 274 | HSPC | NPR3 | 11.6253786 | AVP | 13.2749435 |
| 275 | HSPC | EHD2 | 10.811535 | AC011139.1 | 12.9735958 |
| 276 | HSPC | MYCT1 | 10.6153549 | FREM1 | 12.5390156 |
| 277 | HSPC | PROM1 | 10.5322956 | SHANK3 | 11.6958232 |
| 278 | HSPC | GATA2 | 10.459842 | EHD2 | 11.4878089 |
| 279 | HSPC | NKAIN2 | 10.4418141 | CD34 | 11.338446 |
| 280 | HSPC | ZNF385D | 10.3097804 | MYCT1 | 11.1713819 |
| 281 | HSPC | CXCL11 | 10.1324223 | PROM1 | 11.0952141 |
| 282 | HSPC | SMIM24 | 9.92081812 | SMIM24 | 10.8617509 |
| 283 | HSPC | DYTN | 9.89301581 | AL157895.1 | 10.4585028 |
| 284 | HSPC | SLC8A3 | 9.57318071 | NPR3 | 10.3010384 |
| 285 | HSPC | ADGRG6 | 9.53991752 | HPGDS | 10.1255682 |
| 286 | HSPC | CPXM1 | 9.37554937 | ZNF385D | 10.0378825 |
| 287 | HSPC | TAL1 | 9.22428564 | EMID1 | 9.97623153 |
| 288 | HSPC | GATA2-AS1 | 9.20539667 | APOC1 | 9.86873373 |
| 289 | HSPC | PREX2 | 9.1808816 | HTR1F | 9.84851257 |
| 290 | HSPC | AJ009632.2 | 8.94893902 | CNRIP1 | 9.81759482 |
| 291 | HSPC | ARNTL2-AS1 | 8.9118916 | GATA2-AS1 | 9.78006603 |
| 292 | HSPC | CRHBP | 8.66098007 | GATA1 | 9.27978577 |
| 293 | HSPC | EMID1 | 8.62207154 | SLC8A3 | 9.25744612 |
| 294 | HSPC | C1QTNF4 | 8.60604712 | KIT | 8.99994724 |
| 295 | HSPC | DSG2 | 8.48457898 | NKAIN2 | 8.89641998 |
| 296 | HSPC | HOXA3 | 8.48342938 | AJ009632.2 | 8.89241985 |
| 297 | HSPC | HOXA7 | 8.37671381 | BCAM | 8.82362509 |
| 298 | HSPC | TFPI | 8.3242767 | ADGRG6 | 8.73170298 |
| 299 | HSPC | MPL | 8.30500054 | CPXM1 | 8.66363321 |
| 300 | HSPC | CAVIN1 | 8.30214475 | RYR3 | 8.58798282 |
| 301 | Memory B | TNFRSF13B | 6.40049026 | TNFRSF13B | 6.88965231 |
| 302 | Memory B | SSPN | 5.9630017 | SSPN | 6.80658893 |
| 303 | Memory B | CPNE5 | 5.19695173 | AL355076.2 | 6.21643606 |
| 304 | Memory B | LINC01857 | 5.0881322 | SOX5 | 5.38716364 |
| 305 | Memory B | TLR10 | 4.94504028 | LINC01781 | 5.32906117 |
| 306 | Memory B | CD24 | 4.932165 | CPNE5 | 5.19203455 |
| 307 | Memory B | FCRL5 | 4.71747383 | PPP1R14A | 5.06092092 |
| 308 | Memory B | FCRL2 | 4.70674708 | TLR10 | 4.6007985 |
| 309 | Memory B | MS4A1 | 4.66214347 | CD24 | 4.55920715 |
| 310 | Memory B | FCRLA | 4.62257851 | LINC01857 | 4.5230655 |
| 311 | Memory B | SPIB | 4.60773181 | FCRL5 | 4.48237794 |
| 312 | Memory B | BACE2 | 4.54027651 | OSBPL10 | 4.40850757 |
| 313 | Memory B | BLK | 4.51681431 | CLECL1 | 4.34970717 |
| 314 | Memory B | OSBPL10 | 4.49844949 | FCRL2 | 4.28564932 |
| 315 | Memory B | CD19 | 4.48151788 | RHEX | 4.23294256 |
| 316 | Memory B | BANK1 | 4.40025659 | SPIB | 4.23055082 |
| 317 | Memory B | EBF1 | 4.38203015 | MS4A1 | 4.20140691 |
| 318 | Memory B | CD79A | 4.35877103 | BLK | 4.19983062 |
| 319 | Memory B | PNOC | 4.26972669 | CD1C | 4.19419861 |
| 320 | Memory B | FAM30A | 4.26969785 | BACE2 | 4.16326145 |
| 321 | Memory B | ANGPTL1 | 4.24266331 | BANK1 | 4.08254172 |
| 322 | Memory B | CD1C | 4.23615289 | CD19 | 4.06204445 |
| 323 | Memory B | RHEX | 4.22602749 | POU2AF1 | 4.04085754 |
| 324 | Memory B | VPREB3 | 4.12256273 | FCRLA | 4.0171736 |
| 325 | Memory B | POU2AF1 | 4.11969004 | FAM30A | 4.01464896 |
| 326 | Memory B | PAX5 | 4.05239649 | TSBP1-AS1 | 4.01023917 |
| 327 | Memory B | TNFRSF13C | 4.04254152 | AIM2 | 3.91865829 |
| 328 | Memory B | CLECL1 | 4.01433947 | IGHG2 | 3.91752365 |
| 329 | Memory B | BLNK | 3.91889169 | ANGPTL1 | 3.85735408 |
| 330 | Memory B | CD22 | 3.91844765 | EBF1 | 3.83660493 |
| 331 | MS1 | HP | 4.36340458 | HP | 3.71611108 |
| 332 | MS1 | S100A12 | 3.33798809 | RETN | 3.09987327 |
| 333 | MS1 | RETN | 3.27910869 | S100A12 | 2.85101657 |
| 334 | MS1 | PADI4 | 3.20977523 | IL1R2 | 2.82293999 |
| 335 | MS1 | IL1R2 | 3.09456367 | RNASE2 | 2.80774343 |
| 336 | MS1 | DACH1 | 2.95042489 | MARC1 | 2.6347506 |
| 337 | MS1 | S100A8 | 2.86925938 | S100A8 | 2.60625369 |
| 338 | MS1 | PROK2 | 2.85210005 | FOLR3 | 2.57837295 |
| 339 | MS1 | CLU | 2.78834276 | CLU | 2.56143374 |
| 340 | MS1 | MARC1 | 2.78012978 | MCEMP1 | 2.52971639 |
| 341 | MS1 | MCEMP1 | 2.76808789 | CES1 | 2.46579119 |
| 342 | MS1 | RNASE2 | 2.73066197 | PADI4 | 2.45661339 |
| 343 | MS1 | FOLR3 | 2.61733625 | F5 | 2.32806283 |
| 344 | MS1 | PLBD1 | 2.60472743 | PLBD1 | 2.25666997 |
| 345 | MS1 | F5 | 2.5770134 | ASGR2 | 2.24963426 |
| 346 | MS1 | CES1 | 2.57100504 | CLEC4D | 2.2274881 |
| 347 | MS1 | S100A9 | 2.558455 | ADAMTS2 | 2.18578048 |
| 348 | MS1 | DYSF | 2.50284954 | S100A9 | 2.18262032 |
| 349 | MS1 | QPCT | 2.40296941 | MGST1 | 2.17707136 |
| 350 | MS1 | ASGR2 | 2.37871042 | CRISPLD2 | 2.17392899 |
| 351 | MS1 | CLEC4D | 2.28172741 | CKAP4 | 2.17386203 |
| 352 | MS1 | NFE2 | 2.25389053 | CYP1B1 | 2.16697446 |
| 353 | MS1 | HLX | 2.24923895 | THBS1 | 2.13752097 |
| 354 | MS1 | MGST1 | 2.2479636 | AC020656.1 | 2.12852987 |
| 355 | MS1 | NLRP12 | 2.21622075 | QPCT | 2.08403864 |
| 356 | MS1 | CYP1B1 | 2.21219157 | VCAN | 2.06153114 |
| 357 | MS1 | CKAP4 | 2.20490808 | CCR2 | 2.0526314 |
| 358 | MS1 | CDA | 2.17537752 | AL034397.3 | 2.01168146 |
| 359 | MS1 | LIN7A | 2.1314252 | TPST1 | 2.00315911 |
| 360 | MS1 | PPARG | 2.10662631 | FLT3 | 1.99369278 |
| 361 | Naive B | COL19A1 | 6.41758297 | TCL1A | 8.00689784 |
| 362 | Naive B | TCL1A | 6.3927466 | SLC38A11 | 7.19532624 |
| 363 | Naive B | SLC38A11 | 6.25614235 | COL19A1 | 7.18532978 |
| 364 | Naive B | CD200 | 6.24085811 | IGHD | 7.02182463 |
| 365 | Naive B | IGHD | 6.20892346 | CD200 | 6.72723209 |
| 366 | Naive B | FCER2 | 6.11710538 | FCER2 | 6.48672166 |
| 367 | Naive B | FCRL1 | 6.10804935 | FCRL1 | 6.42001941 |
| 368 | Naive B | LINC00926 | 6.01662662 | IGHV5-78 | 6.26512791 |
| 369 | Naive B | FAM177B | 5.92158167 | LINC00926 | 6.25285631 |
| 370 | Naive B | LINC02397 | 5.91091583 | PCDH9 | 6.22533118 |
| 371 | Naive B | IGHV5-78 | 5.85846462 | VPREB3 | 6.16246263 |
| 372 | Naive B | PCDH9 | 5.75762335 | LINC02397 | 6.09495452 |
| 373 | Naive B | STAG3 | 5.70953154 | NIBAN3 | 6.07345829 |
| 374 | Naive B | LIX1-AS1 | 5.63325692 | FAM177B | 5.96712548 |
| 375 | Naive B | VPREB3 | 5.59773909 | LIX1-AS1 | 5.87840188 |
| 376 | Naive B | NIBAN3 | 5.59441644 | CD22 | 5.84361318 |
| 377 | Naive B | PAX5 | 5.56501401 | STEAP1B | 5.81152144 |
| 378 | Naive B | AFF3 | 5.4378818 | PAX5 | 5.80589572 |
| 379 | Naive B | CXCR5 | 5.39081715 | PTPRK | 5.74358363 |
| 380 | Naive B | IGHM | 5.34212851 | AFF3 | 5.71905333 |
| 381 | Naive B | HLA-DOB | 5.32182757 | STAG3 | 5.71675488 |
| 382 | Naive B | KHDRBS2 | 5.29150355 | CXCR5 | 5.64411553 |
| 383 | Naive B | TNFRSF13C | 5.2730262 | IGHM | 5.62543918 |
| 384 | Naive B | CD22 | 5.2729956 | CD79A | 5.51978706 |
| 385 | Naive B | STEAP1B | 5.25703399 | HLA-DOB | 5.46888557 |
| 386 | Naive B | CD79A | 5.21193038 | TSPAN13 | 5.37853642 |
| 387 | Naive B | TSPAN13 | 5.15869677 | EBF1 | 5.33278659 |
| 388 | Naive B | PTPRK | 5.14112419 | FCRLA | 5.33207101 |
| 389 | Naive B | CD19 | 5.10181746 | TNFRSF13C | 5.29589149 |
| 390 | Naive B | EBF1 | 5.10151729 | CD19 | 5.28700022 |
| 391 | Natural killer | KIR2DL4 | 5.95045844 | KIR2DL1 | 6.29050743 |
| 392 | Natural killer | SH2D1B | 5.67551286 | KIR2DL4 | 6.24109988 |
| 393 | Natural killer | KIR2DL1 | 5.63919211 | SH2D1B | 5.95696661 |
| 394 | Natural killer | PTGDS | 5.2349928 | AKR1C3 | 5.64347734 |
| 395 | Natural killer | KLRC1 | 5.21664688 | PTGDS | 5.33307574 |
| 396 | Natural killer | AKR1C3 | 5.14600675 | KLRC1 | 5.26767884 |
| 397 | Natural killer | KLRF1 | 5.00847038 | LAIR2 | 5.20204687 |
| 398 | Natural killer | LAIR2 | 4.90746731 | KLRF1 | 5.19826043 |
| 399 | Natural killer | MYOM2 | 4.83414037 | KIR3DL1 | 5.17029081 |
| 400 | Natural killer | SPON2 | 4.7803021 | MYOM2 | 4.99828076 |
| 401 | Natural killer | TRDC | 4.76825586 | GNLY | 4.9401634 |
| 402 | Natural killer | KIR3DL1 | 4.71870504 | SPON2 | 4.93070832 |
| 403 | Natural killer | GNLY | 4.64830409 | TRDC | 4.86200192 |
| 404 | Natural killer | CLIC3 | 4.49445519 | NMUR1 | 4.78192069 |
| 405 | Natural killer | NMUR1 | 4.49135873 | CLIC3 | 4.69673565 |
| 40€ | Natural killer | NCR1 | 4.48611671 | CCL3 | 4.69400749 |
| 407 | Natural killer | CD160 | 4.42751622 | NCR1 | 4.68317354 |
| 408 | Natural killer | TMIGD2 | 4.39229504 | GZMB | 4.64404799 |
| 409 | Natural killer | GZMB | 4.36211652 | TMIGD2 | 4.56837804 |
| 410 | Natural killer | XCL2 | 4.30112352 | PRF1 | 4.48865534 |
| 411 | Natural killer | PRF1 | 4.24671485 | FGFBP2 | 4.34885225 |
| 412 | Natural killer | LINC00299 | 4.17639965 | AREG | 4.31862975 |
| 413 | Natural killer | TNFRSF18 | 4.09745433 | XCL2 | 4.29718709 |
| 414 | Natural killer | CCL3 | 4.08670153 | S1PR5 | 4.19308789 |
| 415 | Natural killer | IL2RB | 4.07641295 | LINC00299 | 4.16317948 |
| 416 | Natural killer | FGFBP2 | 4.05329347 | PRSS23 | 4.14968818 |
| 417 | Natural killer | LINGO2 | 4.05131947 | CCL4 | 4.11068715 |
| 418 | Natural killer | S1PR5 | 4.02306966 | IL2RB | 4.08860473 |
| 419 | Natural killer | PRSS23 | 3.9638524 | KLRD1 | 4.06790364 |
| 420 | Natural killer | IL18RAP | 3.93398762 | TRGC1 | 4.05272461 |
| 421 | Plasmablast | IGF1 | 10.0741152 | IGHG1 | 9.78151777 |
| 422 | Plasmablast | BHLHA15 | 9.84885003 | BHLHA15 | 9.77473301 |
| 423 | Plasmablast | IGHA2 | 9.77553349 | IGHA1 | 9.76603243 |
| 424 | Plasmablast | IGHG1 | 9.68670991 | IGF1 | 9.48523875 |
| 425 | Plasmablast | IGHA1 | 9.5103541 | JCHAIN | 9.15148073 |
| 426 | Plasmablast | IGHG4 | 9.15595331 | IGHA2 | 8.97343553 |
| 427 | Plasmablast | JCHAIN | 9.09527266 | MIXL1 | 8.82989347 |
| 428 | Plasmablast | IGHG2 | 8.89685902 | GLDC | 8.59777135 |
| 429 | Plasmablast | IGKC | 8.68150189 | IGKC | 8.42154384 |
| 430 | Plasmablast | GPRC5D | 8.50991223 | IGHV3-23 | 8.40067713 |
| 431 | Plasmablast | IGKV3-20 | 8.49026294 | IGHG2 | 8.35109223 |
| 432 | Plasmablast | IGLC1 | 8.33194746 | IGKV3-20 | 8.20024724 |
| 433 | Plasmablast | GLDC | 8.23640314 | TNFRSF17 | 7.93220322 |
| 434 | Plasmablast | TNFRSF17 | 8.20661998 | IGLC2 | 7.92381798 |
| 435 | Plasmablast | BMP6 | 8.13196214 | MZB1 | 7.91389369 |
| 436 | Plasmablast | IGLV3-1 | 8.08132854 | GPRC5D | 7.77809112 |
| 437 | Plasmablast | MZB1 | 7.99056835 | IGUJ1 | 7.76821655 |
| 438 | Plasmablast | IGLC2 | 7.90396297 | IGLC1 | 7.57671989 |
| 439 | Plasmablast | AC009570.2 | 7.77799387 | BMP6 | 7.52844893 |
| 440 | Plasmablast | IGHG3 | 7.72273924 | DERL3 | 7.2861279 |
| 441 | Plasmablast | MIXL1 | 7.49251639 | AC009570.2 | 7.272683 |
| 442 | Plasmablast | FA2H | 7.4020948 | CAV1 | 7.20496764 |
| 443 | Plasmablast | DERL3 | 7.30032671 | TXNDC5 | 6.87918308 |
| 444 | Plasmablast | TXNDC5 | 6.95633073 | AC104699.1 | 6.81322934 |
| 445 | Plasmablast | KCNN3 | 6.92710705 | ZNF215 | 6.7636576 |
| 446 | Plasmablast | CAV1 | 6.77683067 | FA2H | 6.75303882 |
| 447 | Plasmablast | AC104699.1 | 6.7227436 | IGLC3 | 6.68550256 |
| 448 | Plasmablast | IGKV4-1 | 6.66116589 | ACOXL | 6.5252593 |
| 449 | Plasmablast | IGLC3 | 6.59391581 | KCNN3 | 6.48467879 |
| 450 | Plasmablast | ACOXL | 6.09646541 | PYCR1 | 6.44980162 |
| 451 | Plasmacytoid | AC097375.1 | 14.4018666 | AC097375.1 | 14.9279203 |
| | dendritic cell | | | | |
| 452 | Plasmacytoid | AL513493.1 | 13.3299019 | LRRC26 | 13.9524431 |
| | dendritic cell | | | | |
| 453 | Plasmacytoid | LRRC26 | 13.2526012 | SCT | 13.3366202 |
| | dendritic cell | | | | |
| 454 | Plasmacytoid | KCNK17 | 12.3364586 | AL513493.1 | 12.8012148 |
| | dendritic cell | | | | |
| 455 | Plasmacytoid | KRT5 | 12.1508582 | AC011893.1 | 12.5909393 |
| | dendritic cell | | | | |
| 456 | Plasmacytoid | SCT | 11.9906378 | SHD | 12.443667 |
| | dendritic cell | | | | |
| 457 | Plasmacytoid | SHD | 11.9369146 | KCNK17 | 12.1861433 |
| | dendritic cell | | | | |
| 458 | Plasmacytoid | EPHA2 | 11.0290176 | KRT5 | 12.1292961 |
| | dendritic cell | | | | |
| 459 | Plasmacytoid | LINC01724 | 11.0074634 | EPHA2 | 11.5679092 |
| | dendritic cell | | | | |
| 460 | Plasmacytoid | AC011893.1 | 10.9850575 | LINC01478 | 11.2047157 |
| | dendritic cell | | | | |
| 461 | Plasmacytoid | CLEC4C | 10.9813822 | KCNK10 | 10.7850243 |
| | dendritic cell | | | | |
| 462 | Plasmacytoid | LINC01478 | 10.6372218 | CLEC4C | 10.7317655 |
| | dendritic cell | | | | |
| 463 | Plasmacytoid | COBL | 10.6116629 | COBL | 10.7220218 |
| | dendritic cell | | | | |
| 464 | Plasmacytoid | KCNK10 | 10.3499487 | PROC | 9.64761374 |
| | dendritic cell | | | | |
| 465 | Plasmacytoid | SMIM6 | 9.77375535 | CUX2 | 9.60434103 |
| | dendritic cell | | | | |
| 466 | Plasmacytoid | P3H2 | 9.62714013 | P3H2 | 9.48461258 |
| | dendritic cell | | | | |
| 467 | Plasmacytoid | PTCRA | 9.42254395 | COL26A1 | 9.48240386 |
| | dendritic cell | | | | |
| 468 | Plasmacytoid | PLVAP | 9.32502384 | LINC01226 | 9.30762057 |
| | dendritic cell | | | | |
| 469 | Plasmacytoid | PROC | 9.11624034 | SLC12A3 | 9.11943259 |
| | dendritic cell | | | | |
| 470 | Plasmacytoid | COL26A1 | 9.1045262 | PTPRS | 9.08588027 |
| | dendritic cell | | | | |
| 471 | Plasmacytoid | CUX2 | 8.97044954 | TTC39A | 8.99680766 |
| | dendritic cell | | | | |
| 472 | Plasmacytoid | LILRA4 | 8.94228375 | MAP1A | 8.99323461 |
| | dendritic cell | | | | |
| 473 | Plasmacytoid | SLC12A3 | 8.71043159 | LILRA4 | 8.66288988 |
| | dendritic cell | | | | |
| 474 | Plasmacytoid | PTPRS | 8.66462423 | BEND6 | 8.62817945 |
| | dendritic cell | | | | |
| 475 | Plasmacytoid | LRRC36 | 8.48560954 | LRRC36 | 8.61707772 |
| | dendritic cell | | | | |
| 476 | Plasmacytoid | MAP1A | 8.47953282 | FAM160A1 | 8.59683714 |
| | dendritic cell | | | | |
| 477 | Plasmacytoid | TPM2 | 8.41848212 | LAMP5 | 8.57763355 |
| | dendritic cell | | | | |
| 478 | Plasmacytoid | CYP46A1 | 8.39666483 | CYP46A1 | 8.4895815 |
| | dendritic cell | | | | |
| 479 | Plasmacytoid | LAMP5 | 8.35703714 | PLD4 | 8.4453196 |
| | dendritic cell | | | | |
| 480 | Plasmacytoid | PLD4 | 8.34089087 | TPM2 | 8.28549503 |
| | dendritic cell | | | | |
| 481 | Platelet | GP9 | 10.1828713 | GP9 | 12.3794091 |
| 482 | Platelet | TUBB1 | 9.7936385 | TREML1 | 11.4552433 |
| 483 | Platelet | PPBP | 9.6663975 | PPBP | 11.2668342 |
| 484 | Platelet | TREML1 | 9.57005731 | PF4 | 11.2091842 |
| 485 | Platelet | GP1BB | 9.42272266 | CMTM5 | 10.9763707 |
| 486 | Platelet | PF4 | 8.95002759 | ITGB3 | 10.9348124 |
| 487 | Platelet | GNG11 | 8.74756298 | TUBB1 | 10.9098565 |
| 488 | Platelet | MYL9 | 8.74421144 | GP1BB | 10.7491475 |
| 489 | Platelet | PF4V1 | 8.404107 | CAVIN2 | 10.3495239 |
| 490 | Platelet | CAVIN2 | 8.09281235 | GNG11 | 10.3460942 |
| 491 | Platelet | SPARC | 7.51663501 | MYL9 | 10.2687405 |
| 492 | Platelet | ITGA2B | 7.3819886 | PF4V1 | 10.2165917 |
| 493 | Platelet | MPIG6B | 7.25579453 | ITGA2B | 9.89808894 |
| 494 | Platelet | ACRBP | 4.83062683 | MPIG6B | 9.38767588 |
| 495 | Platelet | NRGN | 4.47895229 | SH3BGRL2 | 8.97948964 |
| 496 | Platelet | PRKAR2B | 4.31550252 | SPARC | 8.96888775 |
| 497 | Platelet | PTGS1 | 4.25821809 | CLEC1B | 8.82837249 |
| 498 | Platelet | MTURN | 3.20348812 | TMEM40 | 8.64349598 |
| 499 | Platelet | SNCA | 2.70446032 | PTCRA | 8.62318241 |
| 500 | Platelet | F13A1 | 2.64062373 | ESAM | 8.44489349 |
| 501 | Platelet | HIST1H2AC | 2.45717371 | C2orf88 | 6.99218868 |
| 502 | Platelet | TPM1 | 2.42522205 | NRGN | 6.73392836 |
| 503 | Platelet | PGRMC1 | 2.22291258 | ACRBP | 6.69642645 |
| 504 | Platelet | RUFY1 | 2.02315003 | CD9 | 6.55015039 |
| 505 | Platelet | EGR1 | 1.9733572 | PRKAR2B | 6.54741996 |
| 506 | Platelet | MAP3K7CL | 1.90860695 | TRIM58 | 5.97338898 |
| 507 | Platelet | TNNT1 | 1.8103211 | PTGS1 | 5.88706841 |
| | Platelet | NRG1 | 1.72989648 | MTURN | 5.36146698 |
| 509 | Platelet | RGS18 | 1.70002798 | BEX3 | 5.11747393 |
| 510 | Platelet | ARHGAP18 | 1.66397712 | SNCA | 4.91104002 |
| 511 | Proliferating T | TYMS | 8.09756022 | TYMS | 8.5485646 |
| 512 | Proliferating T | SPC25 | 7.74824501 | PBK | 8.04893816 |
| 513 | Proliferating T | PBK | 7.73592177 | SPC25 | 7.87436287 |
| 514 | Proliferating T | CDC45 | 7.46417955 | MCM10 | 7.87076532 |
| 515 | Proliferating T | MCM10 | 7.35073654 | CDC45 | 7.59446437 |
| 516 | Proliferating T | CDT1 | 7.2614299 | CDT1 | 7.58507588 |
| 517 | Proliferating T | RRM2 | 7.21889049 | CDC20 | 7.49324469 |
| 518 | Proliferating T | CKAP2L | 7.21682349 | PKMYT1 | 7.46194129 |
| 519 | Proliferating T | CDC20 | 7.20787578 | E2F8 | 7.45220973 |
| 520 | Proliferating T | DLGAP5 | 7.20484134 | DTL | 7.44081452 |
| 521 | Proliferating T | UBE2C | 7.15574954 | CKAP2L | 7.43133632 |
| 522 | Proliferating T | HJURP | 7.15497162 | RRM2 | 7.42130862 |
| 523 | Proliferating T | KIF18B | 7.1444008 | DLGAP5 | 7.36684374 |
| 524 | Proliferating T | CCNA2 | 7.09405026 | HJURP | 7.25266153 |
| 525 | Proliferating T | PKMYT1 | 7.02102298 | KIF18B | 7.20828565 |
| 526 | Proliferating T | ASPM | 7.01140113 | UBE2C | 7.18912466 |
| 527 | Proliferating T | DTL | 6.98442674 | PCLAF | 7.16339912 |
| 528 | Proliferating T | CDCA2 | 6.95942291 | KIFC1 | 7.05598783 |
| 529 | Proliferating T | E2F8 | 6.95605272 | CDK1 | 7.00424944 |
| 530 | Proliferating T | E2F7 | 6.91583607 | GINS2 | 6.99056231 |
| 531 | Proliferating T | GTSE1 | 6.91477722 | CDCA3 | 6.92429952 |
| 532 | Proliferating T | KIFC1 | 6.91439359 | ASPM | 6.90133456 |
| 533 | Proliferating T | CDK1 | 6.7934156 | HIST1H3G | 6.87243656 |
| 534 | Proliferating T | DEPDC1 | 6.73643209 | CDCA2 | 6.86103778 |
| 535 | Proliferating T | TOP2A | 6.68952995 | E2F7 | 6.84530872 |
| 536 | Proliferating T | GINS2 | 6.68487286 | CDC6 | 6.8282104 |
| 537 | Proliferating T | PCLAF | 6.67948817 | FAM111B | 6.81256912 |
| 538 | Proliferating T | KIF20A | 6.67706256 | CCNA2 | 6.79140313 |
| 539 | Proliferating T | HIST1H3G | 6.67484176 | CCNB2 | 6.74425244 |
| 540 | Proliferating T | CDCA5 | 6.66457078 | CDCA5 | 6.73841526 |
| 541 | Regulatory T | FOXP3 | 8.97314848 | FOXP3 | 9.1190867 |
| 542 | Regulatory T | RTKN2 | 6.22582598 | RTKN2 | 6.46639584 |
| 543 | Regulatory T | IL2RA | 5.47364655 | IL2RA | 5.84964706 |
| 544 | Regulatory T | CTLA4 | 5.21395821 | AL136456.1 | 5.69752369 |
| 545 | Regulatory T | AL136456.1 | 5.19134242 | LINC02694 | 5.5631964 |
| 546 | Regulatory T | LINC02694 | 5.00365926 | CTLA4 | 5.51968646 |
| 547 | Regulatory T | CCR4 | 4.42898101 | CCR4 | 4.68868076 |
| 548 | Regulatory T | AC093865.1 | 4.25844494 | AC093865.1 | 4.48445104 |
| 549 | Regulatory T | IKZF2 | 4.08877655 | IKZF2 | 4.47823708 |
| 550 | Regulatory T | PI16 | 3.74954852 | TNFRSF4 | 4.07834959 |
| 551 | Regulatory T | TNFRSF4 | 3.70110548 | ICA1 | 3.79159981 |
| 552 | Regulatory T | TTN | 3.66239703 | RGS1 | 3.78354299 |
| 553 | Regulatory T | TIGIT | 3.38528787 | TTN | 3.71232933 |
| 554 | Regulatory T | CD27 | 3.10189566 | TIGIT | 3.69151116 |
| 555 | Regulatory T | ICOS | 3.08692386 | ICOS | 3.33970234 |
| 556 | Regulatory T | TBC1D4 | 3.02432306 | MAST4 | 3.26188435 |
| 55 | Regulatory T | HPGD | 3.02075458 | DUSP16 | 3.22171992 |
| 558 | Regulatory T | RGS1 | 3.00921904 | LINC00426 | 3.17515433 |
| 559 | Regulatory T | DUSP16 | 2.91953727 | STAM | 3.10233365 |
| 560 | Regulatory T | AQP3 | 2.91567305 | TBC1D4 | 3.08915906 |
| 561 | Regulatory T | LINC00426 | 2.91473408 | CD27 | 3.02057299 |
| 562 | Regulatory T | CD28 | 2.88707109 | PLCL1 | 3.01619869 |
| 563 | Regulatory T | STAM | 2.82754726 | FAAH2 | 2.99051795 |
| 564 | Regulatory T | PLCL1 | 2.8269272 | CD28 | 2.9720528 |
| 565 | Regulatory T | FAAH2 | 2.64369135 | HS3ST3B1 | 2.89446224 |
| 566 | Regulatory T | SIRPG | 2.6270713 | TAFA2 | 2.86672686 |
| 567 | Regulatory T | IL32 | 2.59782378 | AQP3 | 2.85471794 |
| 568 | Regulatory T | TRAC | 2.5721482 | TRAC | 2.80275456 |
| 569 | Regulatory T | ATP8B2 | 2.55893634 | HAPLN3 | 2.7767383 |
| 570 | Regulatory T | TAFA2 | 2.54128676 | ATP8B2 | 2.7491759 |

In an orthogonal approach, gene expression using FindMarkers and the DESeq2 package in R was used to compare cells processed by the two methods to identify differentially expressed genes (Tables 4-5). Table 4 shows the results of an analysis of differential gene expression as assessed by RNA. Table 5 shows the results of an analysis of differential gene expression as assessed by antibody-determined tags (ADT). Of the statistically significant (p<0.05) genes, a substantial (greater than 4) fold-change differences in expression between the two methods was not observed. Most genes with more than a 2-fold expression change were non-coding genes, with the exceptions of the genes CXCL8, FOSB, and JUN genes being slightly up-regulated in Ficoll cells (FIG. 3E). Similar differentially-expressed genes were identified when comparisons were performed at the level of cell types (FIG. 3F), instead of all cells combined. Crucially, no genes that are used to identify cell lineages or cell types were differentially expressed by more than a 2-fold change. Pathway analysis could not be performed due to the sparse number of substantially differentially expressed genes. However, immediate early genes are a class of genes commonly transiently upregulated in many types of cells as a primary response to a variety of stimuli, the presence of the immediate early genes JUN and FOSB may suggest an early response to ex-vivo stimulation in Ficoll cells (27, 28).

TABLE 4
Analysis of differential gene expression as assessed by RNA

| | | avg_log2FC | | | | | |
|---|---|---|---|---|---|---|---|
| | | positive = | | | | | significant + |
| | | enriched in | | | | | fold change |
| | | Ficoll, | | | | significant | (p_val_adjusted < |
| | | negative = | | | | (p_val_ad | 0.05 and |
| | | enriched in | | | | justed < | abs(avg_log2FC) > |
| | p_val | Cryo-PRO | p_val_adjusted | cell type | gene | 0.05?) | 1 ?) |
| 1 | 5.81E−31 | 0.62713714 | 1.43E−26 | Monocyte | AL137060.3 | TRUE | FALSE |
| 2 | 7.47E−30 | 0.66392036 | 1.84E−25 | Monocyte | MPP7-DT | TRUE | FALSE |
| 3 | 5.46E−27 | 1.38481427 | 1.34E−22 | Monocyte | HLX-AS1 | TRUE | TRUE |
| 4 | 2.71E−24 | 0.62382477 | 6.67E−20 | Monocyte | AL450992.1 | TRUE | FALSE |
| 5 | 9.30E−23 | 1.39817136 | 2.29E−18 | Monocyte | MYOSLID | TRUE | TRUE |
| 6 | 7.92E−21 | 0.69323853 | 1.95E−16 | Monocyte | JARID2-AS1 | TRUE | FALSE |
| 7 | 5.08E−20 | 0.54627422 | 1.25E−15 | Monocyte | LINC02669 | TRUE | FALSE |
| 8 | 7.68E−20 | 0.35893318 | 1.89E−15 | Monocyte | KLF3-AS1 | TRUE | FALSE |
| 9 | 8.96E−20 | 0.98552086 | 2.20E−15 | Monocyte | AC104695.2 | TRUE | FALSE |
| 10 | 1.04E−19 | 0.92067044 | 2.56E−15 | Monocyte | SPAG5-AS1 | TRUE | FALSE |
| 11 | 3.09E−19 | 0.69066609 | 7.61E−15 | Monocyte | AC017083.1 | TRUE | FALSE |
| 12 | 3.32E−19 | 0.38436371 | 8.17E−15 | Monocyte | AC006994.2 | TRUE | FALSE |
| 13 | 8.18E−19 | 0.62337117 | 2.01E−14 | Monocyte | AC010864.1 | TRUE | FALSE |
| 14 | 1.19E−18 | 1.22332754 | 2.92E−14 | Monocyte | SIAH2-AS1 | TRUE | TRUE |
| 15 | 1.27E−18 | 0.57587697 | 3.14E−14 | Monocyte | AL359711.2 | TRUE | FALSE |
| 16 | 1.48E−18 | 1.05337918 | 3.64E−14 | Monocyte | AL158801.2 | TRUE | TRUE |
| 17 | 5.56E−18 | 0.89386397 | 1.37E−13 | Monocyte | KLF4 | TRUE | FALSE |
| 18 | 9.70E−18 | 0.93168088 | 2.39E−13 | Monocyte | LINC01220 | TRUE | FALSE |
| 19 | 3.86E−17 | 0.46770267 | 9.50E−13 | Monocyte | AL627171.1 | TRUE | FALSE |
| 20 | 5.12E−17 | 0.6177506 | 1.26E−12 | Monocyte | AL353719.1 | TRUE | FALSE |
| 21 | 1.15E−16 | 0.66779413 | 2.83E−12 | Monocyte | AC023509.3 | TRUE | FALSE |
| 22 | 1.43E−16 | 1.05922115 | 3.52E−12 | Monocyte | AC091271.1 | TRUE | TRUE |
| 23 | 1.62E−16 | 0.61683166 | 3.98E−12 | Monocyte | UBAC2-AS1 | TRUE | FALSE |
| 24 | 2.14E−16 | 0.35744655 | 5.27E−12 | Monocyte | AL135791.1 | TRUE | FALSE |
| 25 | 2.17E−16 | 0.85351849 | 5.34E−12 | Monocyte | AC079305.1 | TRUE | FALSE |
| 26 | 2.18E−16 | 0.70527816 | 5.36E−12 | Monocyte | AL356512.1 | TRUE | FALSE |
| 27 | 3.42E−16 | 0.96074275 | 8.41E−12 | Monocyte | AC022217.3 | TRUE | FALSE |
| 28 | 3.51E−16 | 0.39609373 | 8.63E−12 | Monocyte | AC022182.1 | TRUE | FALSE |
| 29 | 5.58E−16 | 0.52875562 | 1.37E−11 | Monocyte | AC023790.2 | TRUE | FALSE |
| 30 | 5.84E−16 | 0.34583226 | 1.44E−11 | Monocyte | AC091214.1 | TRUE | FALSE |
| 31 | 6.49E−16 | 0.94434494 | 1.60E−11 | Monocyte | AC025171.3 | TRUE | FALSE |
| 32 | 1.66E−15 | 1.02867705 | 4.07E−11 | Monocyte | NR4A2 | TRUE | TRUE |
| 33 | 1.98E−15 | 0.69395052 | 4.88E−11 | Monocyte | AC110741.1 | TRUE | FALSE |
| 34 | 2.55E−15 | 0.52851164 | 6.27E−11 | Monocyte | AC069431.1 | TRUE | FALSE |
| 35 | 3.20E−15 | 0.45576304 | 7.87E−11 | Monocyte | EZR-AS1 | TRUE | FALSE |
| 36 | 5.80E−15 | 1.13783091 | 1.43E−10 | Monocyte | AC008440.1 | TRUE | TRUE |
| 37 | 5.83E−15 | 1.29992201 | 1.44E−10 | Monocyte | AC020911.2 | TRUE | TRUE |
| 38 | 8.46E−15 | 0.36141899 | 2.08E−10 | Monocyte | AL512791.2 | TRUE | FALSE |
| 39 | 8.74E−15 | 0.65591397 | 2.15E−10 | Monocyte | AL139106.1 | TRUE | FALSE |
| 40 | 1.28E−14 | 0.66840335 | 3.15E−10 | Monocyte | HOOK2 | TRUE | FALSE |
| 41 | 1.32E−14 | 0.30763045 | 3.24E−10 | Monocyte | AL391832.4 | TRUE | FALSE |
| 42 | 1.56E−14 | 1.02057187 | 3.85E−10 | Monocyte | EFNA5 | TRUE | TRUE |
| 43 | 1.63E−14 | 0.29449847 | 4.01E−10 | Monocyte | AC007365.1 | TRUE | FALSE |
| 44 | 1.68E−14 | 0.2566993 | 4.13E−10 | Monocyte | AC123777.1 | TRUE | FALSE |
| 45 | 2.83E−14 | 0.20220327 | 6.97E−10 | Monocyte | AL627422.2 | TRUE | FALSE |
| 46 | 3.11E−14 | 0.60442943 | 7.65E−10 | Monocyte | PIGA | TRUE | FALSE |
| 47 | 3.12E−14 | 0.39091215 | 7.67E−10 | Monocyte | CTH | TRUE | FALSE |
| 48 | 3.96E−14 | 0.82842533 | 9.74E−10 | Monocyte | AC007569.1 | TRUE | FALSE |
| 49 | 4.62E−14 | 0.7616629 | 1.14E−09 | Monocyte | COQ7 | TRUE | FALSE |
| 50 | 9.20E−14 | 1.31943139 | 2.26E−09 | Monocyte | ATP2B1-AS1 | TRUE | TRUE |
| 51 | 1.02E−13 | 0.33691771 | 2.51E−09 | Monocyte | AL138895.1 | TRUE | FALSE |
| 52 | 1.43E−13 | 0.87007023 | 3.52E−09 | Monocyte | BHLHE40-AS1 | TRUE | FALSE |
| 53 | 1.48E−13 | −0.3689517 | 3.63E−09 | Monocyte | UHMK1 | TRUE | FALSE |
| 54 | 1.49E−13 | 0.84845339 | 3.67E−09 | Monocyte | EFCAB2 | TRUE | FALSE |
| 55 | 1.54E−13 | 0.23111296 | 3.78E−09 | Monocyte | AL022069.1 | TRUE | FALSE |
| 56 | 1.84E−13 | 0.75526361 | 4.52E−09 | Monocyte | AL138720.1 | TRUE | FALSE |
| 57 | 2.08E−13 | 0.37662499 | 5.12E−09 | Monocyte | AL121574.1 | TRUE | FALSE |
| 58 | 2.14E−13 | 0.14238521 | 5.26E−09 | Monocyte | LINC00484 | TRUE | FALSE |
| 59 | 2.85E−13 | 0.37078816 | 7.02E−09 | Monocyte | TULP2 | TRUE | FALSE |
| 60 | 3.21E−13 | 0.46887914 | 7.90E−09 | Monocyte | AC012640.2 | TRUE | FALSE |
| 61 | 4.90E−13 | 0.37375258 | 1.21E−08 | Monocyte | YPEL5 | TRUE | FALSE |
| 62 | 5.04E−13 | −0.446359 | 1.24E−08 | Monocyte | OIP5-AS1 | TRUE | FALSE |
| 63 | 5.34E−13 | 0.26022642 | 1.31E−08 | Monocyte | AL139393.3 | TRUE | FALSE |
| 64 | 6.82E−13 | 0.65357312 | 1.68E−08 | Monocyte | AL121601.1 | TRUE | FALSE |
| 65 | 7.23E−13 | 0.38924277 | 1.78E−08 | Monocyte | AC005355.1 | TRUE | FALSE |
| 66 | 1.05E−12 | 0.26029162 | 2.58E−08 | Monocyte | USP12-AS2 | TRUE | FALSE |
| 67 | 1.15E−12 | 0.26612572 | 2.83E−08 | Monocyte | AL353147.1 | TRUE | FALSE |
| 68 | 1.45E−12 | 0.33614377 | 3.57E−08 | Monocyte | LINC01800 | TRUE | FALSE |
| 69 | 1.51E−12 | 0.17044404 | 3.72E−08 | Monocyte | AC012485.3 | TRUE | FALSE |
| 70 | 1.67E−12 | 0.61249575 | 4.10E−08 | Monocyte | ZNF487 | TRUE | FALSE |
| 71 | 1.84E−12 | 0.58594499 | 4.52E−08 | Monocyte | LINC02265 | TRUE | FALSE |
| 72 | 1.95E−12 | 0.16986636 | 4.79E−08 | Monocyte | SLC25A30-AS1 | TRUE | FALSE |
| 73 | 2.11E−12 | 0.89542482 | 5.20E−08 | Monocyte | FAM234B | TRUE | FALSE |
| 74 | 2.35E−12 | 0.8576153 | 5.79E−08 | Monocyte | AL499604.1 | TRUE | FALSE |
| 75 | 2.36E−12 | 0.404203 | 5.82E−08 | Monocyte | AC006511.6 | TRUE | FALSE |
| 76 | 2.48E−12 | 0.36530595 | 6.10E−08 | Monocyte | FAM229B | TRUE | FALSE |
| 77 | 2.76E−12 | 0.33103391 | 6.79E−08 | Monocyte | GCC2-AS1 | TRUE | FALSE |
| 78 | 3.02E−12 | 0.45470969 | 7.44E−08 | Monocyte | GNAT2 | TRUE | FALSE |
| 79 | 3.21E−12 | 0.34311969 | 7.90E−08 | Monocyte | SPART-AS1 | TRUE | FALSE |
| 80 | 3.59E−12 | 0.40412789 | 8.83E−08 | Monocyte | AC083880.1 | TRUE | FALSE |
| 81 | 3.73E−12 | 0.29312723 | 9.17E−08 | Monocyte | AC073195.1 | TRUE | FALSE |
| 82 | 4.11E−12 | 0.74605432 | 1.01E−07 | Monocyte | GABARAPL1 | TRUE | FALSE |
| 83 | 4.18E−12 | 0.34041039 | 1.03E−07 | Monocyte | AC005332.1 | TRUE | FALSE |
| 84 | 4.58E−12 | 0.24124244 | 1.13E−07 | Monocyte | SCN11A | TRUE | FALSE |
| 85 | 5.07E−12 | 0.6787079 | 1.25E−07 | Monocyte | PHLDA1 | TRUE | FALSE |
| 86 | 5.45E−12 | −0.4002032 | 1.34E−07 | Monocyte | CBL | TRUE | FALSE |
| 87 | 6.28E−12 | −0.5178074 | 1.55E−07 | Monocyte | AC007406.5 | TRUE | FALSE |
| 88 | 6.71E−12 | −0.5882935 | 1.65E−07 | Monocyte | ZNF780B | TRUE | FALSE |
| 89 | 7.81E−12 | 0.5166343 | 1.92E−07 | Monocyte | AMZ1 | TRUE | FALSE |
| 90 | 8.95E−12 | −0.2488527 | 2.20E−07 | Monocyte | IGIP | TRUE | FALSE |
| 91 | 9.85E−12 | 0.35821788 | 2.42E−07 | Monocyte | UBE2R2-AS1 | TRUE | FALSE |
| 92 | 1.01E−11 | 0.30454197 | 2.47E−07 | Monocyte | AC093462.1 | TRUE | FALSE |
| 93 | 1.01E−11 | 0.62495511 | 2.49E−07 | Monocyte | AC092431.1 | TRUE | FALSE |
| 94 | 1.25E−11 | 0.19932341 | 3.07E−07 | Monocyte | AC006207.1 | TRUE | FALSE |
| 95 | 1.33E−11 | 0.50230818 | 3.28E−07 | Monocyte | LINC00513 | TRUE | FALSE |
| 96 | 1.38E−11 | 0.21013604 | 3.41E−07 | Monocyte | UBE2L5 | TRUE | FALSE |
| 97 | 1.41E−11 | 0.65169731 | 3.47E−07 | Monocyte | GSG1 | TRUE | FALSE |
| 98 | 1.41E−11 | 0.80144426 | 3.47E−07 | Monocyte | OTUD1 | TRUE | FALSE |
| 99 | 1.50E−11 | −0.392065 | 3.69E−07 | Monocyte | EXOC5 | TRUE | FALSE |
| 100 | 1.55E−11 | 0.45824867 | 3.82E−07 | Monocyte | HIST1H2BN | TRUE | FALSE |
| 101 | 1.98E−11 | 0.53217334 | 4.87E−07 | Monocyte | HIST1H3A | TRUE | FALSE |
| 102 | 2.20E−11 | 0.27426278 | 5.40E−07 | Monocyte | BX323046.1 | TRUE | FALSE |
| 103 | 2.27E−11 | 0.28818315 | 5.60E−07 | Monocyte | ETFBKMT | TRUE | FALSE |
| 104 | 2.57E−11 | 0.1648013 | 6.32E−07 | Monocyte | AL157756.1 | TRUE | FALSE |
| 105 | 2.88E−11 | 0.258437 | 7.08E−07 | Monocyte | AC112236.2 | TRUE | FALSE |
| 106 | 2.96E−11 | 0.42471597 | 7.28E−07 | Monocyte | AC010173.1 | TRUE | FALSE |
| 107 | 3.33E−11 | 0.29414282 | 8.19E−07 | Monocyte | AL133523.1 | TRUE | FALSE |
| 108 | 4.01E−11 | 0.89091062 | 9.86E−07 | Monocyte | PPP1R15A | TRUE | FALSE |
| 109 | 4.35E−11 | 1.39046476 | 1.07E−06 | Monocyte | AC007032.1 | TRUE | TRUE |
| 110 | 4.54E−11 | 0.5350481 | 1.12E−06 | Monocyte | AC004854.2 | TRUE | FALSE |
| 111 | 4.77E−11 | 0.85328135 | 1.17E−06 | Monocyte | HECW2 | TRUE | FALSE |
| 112 | 4.97E−11 | 0.56017054 | 1.22E−06 | Monocyte | VIM-AS1 | TRUE | FALSE |
| 113 | 5.15E−11 | 0.15999339 | 1.27E−06 | Monocyte | RNF43 | TRUE | FALSE |
| 114 | 5.38E−11 | −0.4998887 | 1.32E−06 | Monocyte | ZBTB37 | TRUE | FALSE |
| 115 | 5.60E−11 | 0.41286473 | 1.38E−06 | Monocyte | AL137779.2 | TRUE | FALSE |
| 116 | 5.80E−11 | 0.19440028 | 1.43E−06 | Monocyte | BX323046.2 | TRUE | FALSE |
| 117 | 6.01E−11 | 0.33667886 | 1.48E−06 | Monocyte | PPIL6 | TRUE | FALSE |
| 118 | 6.34E−11 | 0.39041601 | 1.56E−06 | Monocyte | AP001363.2 | TRUE | FALSE |
| 119 | 7.13E−11 | 0.68165081 | 1.76E−06 | Monocyte | AC072022.2 | TRUE | FALSE |
| 120 | 7.46E−11 | 0.47068287 | 1.84E−06 | Monocyte | AL021396.1 | TRUE | FALSE |
| 121 | 7.54E−11 | 0.78383824 | 1.86E−06 | Monocyte | KLHL15 | TRUE | FALSE |
| 122 | 7.85E−11 | 0.23280884 | 1.93E−06 | Monocyte | CNGA4 | TRUE | FALSE |
| 123 | 8.04E−11 | 0.56471784 | 1.98E−06 | Monocyte | DNAJB5-DT | TRUE | FALSE |
| 124 | 8.80E−11 | 0.97451378 | 2.17E−06 | Monocyte | AC011444.3 | TRUE | FALSE |
| 125 | 1.01E−10 | 0.3264008 | 2.49E−06 | Monocyte | AL157394.3 | TRUE | FALSE |
| 126 | 1.15E−10 | −0.3026062 | 2.82E−06 | Monocyte | CNOT6 | TRUE | FALSE |
| 127 | 1.15E−10 | 0.62332272 | 2.84E−06 | Monocyte | CUBN | TRUE | FALSE |
| 128 | 1.23E−10 | 0.27833156 | 3.03E−06 | Monocyte | TERC | TRUE | FALSE |
| 129 | 1.43E−10 | 0.31919937 | 3.53E−06 | Monocyte | C17orf64 | TRUE | FALSE |
| 130 | 1.49E−10 | 0.22659654 | 3.68E−06 | Monocyte | AL035661.2 | TRUE | FALSE |
| 131 | 1.50E−10 | 0.17612354 | 3.70E−06 | Monocyte | HSF1 | TRUE | FALSE |
| 132 | 1.55E−10 | 0.47173376 | 3.81E−06 | Monocyte | CBX4 | TRUE | FALSE |
| 133 | 1.67E−10 | 0.50323568 | 4.11E−06 | Monocyte | AP003717.4 | TRUE | FALSE |
| 134 | 2.03E−10 | 0.54607891 | 4.99E−06 | Monocyte | AL024507.2 | TRUE | FALSE |
| 135 | 2.04E−10 | 0.31599636 | 5.01E−06 | Monocyte | AC092343.1 | TRUE | FALSE |
| 136 | 2.15E−10 | 0.24416357 | 5.28E−06 | Monocyte | C18orf65 | TRUE | FALSE |
| 137 | 2.22E−10 | 0.3988406 | 5.46E−06 | Monocyte | AL022069.3 | TRUE | FALSE |
| 138 | 2.30E−10 | 1.20126943 | 5.67E−06 | Monocyte | RGCC | TRUE | TRUE |
| 139 | 2.37E−10 | 1.15737459 | 5.83E−06 | Monocyte | MIR222HG | TRUE | TRUE |
| 140 | 2.38E−10 | 0.48737141 | 5.85E−06 | Monocyte | HIST1H2BC | TRUE | FALSE |
| 141 | 2.52E−10 | 0.22222002 | 6.19E−06 | Monocyte | ENSA | TRUE | FALSE |
| 142 | 2.69E−10 | 0.30726813 | 6.61E−06 | Monocyte | SPAG6 | TRUE | FALSE |
| 143 | 2.80E−10 | 0.38778259 | 6.88E−06 | Monocyte | CAMTA1-DT | TRUE | FALSE |
| 144 | 2.86E−10 | 0.45683422 | 7.03E−06 | Monocyte | SMG7-AS1 | TRUE | FALSE |
| 145 | 3.17E−10 | 0.22256498 | 7.80E−06 | Monocyte | AC124242.1 | TRUE | FALSE |
| 146 | 4.19E−10 | 0.17973985 | 1.03E−05 | Monocyte | AL022329.1 | TRUE | FALSE |
| 147 | 4.21E−10 | 0.4030284 | 1.04E−05 | Monocyte | AC087623.2 | TRUE | FALSE |
| 148 | 4.27E−10 | −0.355456 | 1.05E−05 | Monocyte | TLR1 | TRUE | FALSE |
| 149 | 4.58E−10 | 0.84552509 | 1.13E−05 | Monocyte | DRAIC | TRUE | FALSE |
| 150 | 4.62E−10 | 0.20364411 | 1.14E−05 | Monocyte | LINC01126 | TRUE | FALSE |
| 151 | 4.79E−10 | 0.30750438 | 1.18E−05 | Monocyte | GASAL1 | TRUE | FALSE |
| 152 | 4.84E−10 | 0.31804342 | 1.19E−05 | Monocyte | C6orf52 | TRUE | FALSE |
| 153 | 5.30E−10 | 0.93574599 | 1.30E−05 | Monocyte | AL512603.2 | TRUE | FALSE |
| 154 | 6.02E−10 | 0.34215329 | 1.48E−05 | Monocyte | SBDS | TRUE | FALSE |
| 155 | 6.64E−10 | −0.3863179 | 1.63E−05 | Monocyte | TFCP2 | TRUE | FALSE |
| 156 | 6.71E−10 | 0.21775105 | 1.65E−05 | Monocyte | PXT1 | TRUE | FALSE |
| 157 | 6.76E−10 | 0.55317344 | 1.66E−05 | Monocyte | ZEB2-AS1 | TRUE | FALSE |
| 158 | 7.11E−10 | 0.28710582 | 1.75E−05 | Monocyte | AL158071.1 | TRUE | FALSE |
| 159 | 7.23E−10 | 0.18631228 | 1.78E−05 | Monocyte | AC012360.1 | TRUE | FALSE |
| 160 | 7.38E−10 | 0.35727787 | 1.82E−05 | Monocyte | HIST1H4A | TRUE | FALSE |
| 161 | 7.38E−10 | 0.14833355 | 1.82E−05 | Monocyte | AC005083.1 | TRUE | FALSE |
| 162 | 7.39E−10 | 0.26261358 | 1.82E−05 | Monocyte | SLC19A2 | TRUE | FALSE |
| 163 | 7.46E−10 | 0.5566801 | 1.84E−05 | Monocyte | AC020765.2 | TRUE | FALSE |
| 164 | 8.17E−10 | 0.50432411 | 2.01E−05 | Monocyte | SPAG1 | TRUE | FALSE |
| 165 | 8.91E−10 | −0.3911264 | 2.19E−05 | Monocyte | TET3 | TRUE | FALSE |
| 166 | 9.20E−10 | 0.14226395 | 2.26E−05 | Monocyte | MAPK6-DT | TRUE | FALSE |
| 167 | 9.44E−10 | −0.2828412 | 2.32E−05 | Monocyte | TRAPPC6B | TRUE | FALSE |
| 168 | 1.07E−09 | 0.172118 | 2.63E−05 | Monocyte | IQCJ-SCHIP1 | TRUE | FALSE |
| 169 | 1.17E−09 | 0.2000379 | 2.87E−05 | Monocyte | AC093677.2 | TRUE | FALSE |
| 170 | 1.23E−09 | 0.29152296 | 3.02E−05 | Monocyte | AC002456.1 | TRUE | FALSE |
| 171 | 1.23E−09 | 0.27844106 | 3.03E−05 | Monocyte | POPDC2 | TRUE | FALSE |
| 172 | 1.50E−09 | 0.4414296 | 3.69E−05 | Monocyte | AL451085.1 | TRUE | FALSE |
| 173 | 1.58E−09 | 0.33448429 | 3.89E−05 | Monocyte | IGLV10-54 | TRUE | FALSE |
| 174 | 1.61E−09 | 0.29398452 | 3.96E−05 | Monocyte | AC138304.1 | TRUE | FALSE |
| 175 | 1.61E−09 | 0.42981365 | 3.97E−05 | Monocyte | AC105384.1 | TRUE | FALSE |
| 176 | 1.69E−09 | −0.3289428 | 4.17E−05 | Monocyte | DCP2 | TRUE | FALSE |
| 177 | 1.71E−09 | −0.4039145 | 4.20E−05 | Monocyte | ANKRD30BL | TRUE | FALSE |
| 178 | 1.89E−09 | 0.40846585 | 4.65E−05 | Monocyte | AC093635.1 | TRUE | FALSE |
| 179 | 1.98E−09 | 0.33218367 | 4.88E−05 | Monocyte | TM4SF20 | TRUE | FALSE |
| 180 | 2.00E−09 | 0.19351849 | 4.92E−05 | Monocyte | AC073352.2 | TRUE | FALSE |
| 181 | 2.05E−09 | 1.57973797 | 5.05E−05 | Monocyte | JUN | TRUE | TRUE |
| 182 | 2.07E−09 | 0.21927625 | 5.08E−05 | Monocyte | MIR17HG | TRUE | FALSE |
| 183 | 2.22E−09 | 0.42710033 | 5.45E−05 | Monocyte | AC072061.1 | TRUE | FALSE |
| 184 | 2.23E−09 | 0.20933496 | 5.50E−05 | Monocyte | AL360227.1 | TRUE | FALSE |
| 185 | 2.36E−09 | −0.3577325 | 5.81E−05 | Monocyte | AC114781.2 | TRUE | FALSE |
| 186 | 2.46E−09 | 0.42576149 | 6.06E−05 | Monocyte | HIST1H2AL | TRUE | FALSE |
| 187 | 2.60E−09 | 0.2902988 | 6.39E−05 | Monocyte | METTL6 | TRUE | FALSE |
| 188 | 3.45E−09 | 1.22781148 | 8.48E−05 | Monocyte | AL691403.1 | TRUE | TRUE |
| 189 | 3.59E−09 | 0.24574168 | 8.82E−05 | Monocyte | AC008115.1 | TRUE | FALSE |
| 190 | 3.73E−09 | −0.4337075 | 9.18E−05 | Monocyte | CEPT1 | TRUE | FALSE |
| 191 | 3.90E−09 | 0.41705464 | 9.59E−05 | Monocyte | CASP9 | TRUE | FALSE |
| 192 | 4.03E−09 | 0.37762263 | 9.93E−05 | Monocyte | MAPRE2 | TRUE | FALSE |
| 193 | 4.27E−09 | 0.42932697 | 0.00010514 | Monocyte | TOB1-AS1 | TRUE | FALSE |
| 194 | 4.41E−09 | −0.2802315 | 0.00010849 | Monocyte | STX7 | TRUE | FALSE |
| 195 | 4.43E−09 | 0.15611362 | 0.00010894 | Monocyte | HIF1A-AS1 | TRUE | FALSE |
| 196 | 4.49E−09 | 0.22553263 | 0.00011048 | Monocyte | SIRT2 | TRUE | FALSE |
| 197 | 4.58E−09 | 0.3779372 | 0.00011259 | Monocyte | NANOS3 | TRUE | FALSE |
| 198 | 4.59E−09 | 0.18387201 | 0.00011292 | Monocyte | AL353135.1 | TRUE | FALSE |
| 199 | 4.64E−09 | 0.3035927 | 0.00011416 | Monocyte | AKIRIN2 | TRUE | FALSE |
| 200 | 4.68E−09 | 0.2926825 | 0.00011517 | Monocyte | AL096677.1 | TRUE | FALSE |
| 201 | 4.73E−09 | 0.33714821 | 0.00011647 | Monocyte | AL355490.2 | TRUE | FALSE |
| 202 | 5.23E−09 | 0.38467986 | 0.00012863 | Monocyte | RASD1 | TRUE | FALSE |
| 203 | 5.41E−09 | 0.3704196 | 0.00013315 | Monocyte | LINC02776 | TRUE | FALSE |
| 204 | 5.58E−09 | 1.03833522 | 0.00013731 | Monocyte | AC020916.1 | TRUE | TRUE |
| 205 | 5.76E−09 | 0.21336508 | 0.00014181 | Monocyte | AL590096.1 | TRUE | FALSE |
| 206 | 5.89E−09 | 0.20460305 | 0.00014483 | Monocyte | CH25H | TRUE | FALSE |
| 207 | 6.12E−09 | 0.76335709 | 0.00015048 | Monocyte | TSPYL2 | TRUE | FALSE |
| 208 | 6.13E−09 | 0.13710163 | 0.00015085 | Monocyte | AL591846.2 | TRUE | FALSE |
| 209 | 6.48E−09 | 0.68324147 | 0.00015948 | Monocyte | TEX41 | TRUE | FALSE |
| 210 | 6.67E−09 | 0.2789729 | 0.00016418 | Monocyte | YWHAQ | TRUE | FALSE |
| 211 | 7.08E−09 | −0.2763953 | 0.00017432 | Monocyte | YIPF4 | TRUE | FALSE |
| 212 | 7.25E−09 | 0.35601338 | 0.0001785 | Monocyte | OSGIN2 | TRUE | FALSE |
| 213 | 7.47E−09 | 0.66374609 | 0.00018385 | Monocyte | CITED2 | TRUE | FALSE |
| 214 | 7.68E−09 | 0.11768178 | 0.00018886 | Monocyte | AC132872.2 | TRUE | FALSE |
| 215 | 7.88E−09 | 0.36127996 | 0.00019395 | Monocyte | LINC01010 | TRUE | FALSE |
| 216 | 7.91E−09 | 0.29189501 | 0.00019474 | Monocyte | CTNNAL1 | TRUE | FALSE |
| 217 | 8.07E−09 | 0.28750886 | 0.00019859 | Monocyte | YME1L1 | TRUE | FALSE |
| 218 | 8.19E−09 | 0.22098631 | 0.00020159 | Monocyte | AC096577.1 | TRUE | FALSE |
| 219 | 8.59E−09 | 0.47296677 | 0.00021132 | Monocyte | LINC02541 | TRUE | FALSE |
| 220 | 8.74E−09 | 0.19252597 | 0.00021517 | Monocyte | TMEM52B | TRUE | FALSE |
| 221 | 8.82E−09 | 0.73162925 | 0.0002171 | Monocyte | Z99127.4 | TRUE | FALSE |
| 222 | 8.97E−09 | −0.2681537 | 0.00022079 | Monocyte | ZBTB41 | TRUE | FALSE |
| 223 | 9.51E−09 | −0.4025938 | 0.000234 | Monocyte | ABHD18 | TRUE | FALSE |
| 224 | 9.96E−09 | 0.35594734 | 0.00024501 | Monocyte | AC123595.1 | TRUE | FALSE |
| 225 | 1.12E−08 | −0.343583 | 0.00027589 | Monocyte | UBE2W | TRUE | FALSE |
| 226 | 1.17E−08 | 0.38861034 | 0.00028884 | Monocyte | MAP1LC3B2 | TRUE | FALSE |
| 227 | 1.25E−08 | 0.70117887 | 0.00030704 | Monocyte | ZFX-AS1 | TRUE | FALSE |
| 228 | 1.27E−08 | 0.65206092 | 0.00031329 | Monocyte | AF213884.3 | TRUE | FALSE |
| 229 | 1.41E−08 | 0.45982461 | 0.00034581 | Monocyte | PTGER2 | TRUE | FALSE |
| 230 | 1.42E−08 | −0.3067832 | 0.00034841 | Monocyte | ZNF518A | TRUE | FALSE |
| 231 | 1.45E−08 | −0.3986098 | 0.00035732 | Monocyte | ZNF251 | TRUE | FALSE |
| 232 | 1.46E−08 | 0.16999372 | 0.00035878 | Monocyte | AC007686.4 | TRUE | FALSE |
| 233 | 1.53E−08 | 0.16197645 | 0.00037707 | Monocyte | ZSWIM2 | TRUE | FALSE |
| 234 | 1.57E−08 | 0.37637735 | 0.00038518 | Monocyte | AC144652.1 | TRUE | FALSE |
| 235 | 1.58E−08 | −0.3379171 | 0.00038932 | Monocyte | TAOK1 | TRUE | FALSE |
| 236 | 1.61E−08 | 0.22496788 | 0.00039535 | Monocyte | AC013400.1 | TRUE | FALSE |
| 237 | 1.63E−08 | 0.4227856 | 0.00040206 | Monocyte | AC092164.1 | TRUE | FALSE |
| 238 | 1.65E−08 | −0.3440188 | 0.00040543 | Monocyte | ZNF175 | TRUE | FALSE |
| 239 | 1.66E−08 | −0.4107962 | 0.00040871 | Monocyte | CYB561D1 | TRUE | FALSE |
| 240 | 1.67E−08 | 0.93076387 | 0.00041049 | Monocyte | AF111167.1 | TRUE | FALSE |
| 241 | 1.68E−08 | 0.17354311 | 0.00041276 | Monocyte | AC008897.2 | TRUE | FALSE |
| 242 | 1.69E−08 | 0.62062404 | 0.00041534 | Monocyte | TOB1 | TRUE | FALSE |
| 243 | 1.92E−08 | 0.26371636 | 0.00047278 | Monocyte | LINC02539 | TRUE | FALSE |
| 244 | 1.98E−08 | −0.2987049 | 0.00048676 | Monocyte | NR2C2 | TRUE | FALSE |
| 245 | 2.08E−08 | 0.32660486 | 0.00051251 | Monocyte | ZNF821 | TRUE | FALSE |
| 246 | 2.09E−08 | 0.14082978 | 0.00051545 | Monocyte | DYRK3 | TRUE | FALSE |
| 247 | 2.14E−08 | −0.3194533 | 0.00052632 | Monocyte | ELK4 | TRUE | FALSE |
| 248 | 2.17E−08 | 0.51864901 | 0.00053401 | Monocyte | AC104984.2 | TRUE | FALSE |
| 249 | 2.21E−08 | 0.46865116 | 0.00054258 | Monocyte | ITPRIP | TRUE | FALSE |
| 250 | 2.31E−08 | 0.15206797 | 0.00056814 | Monocyte | OSR2 | TRUE | FALSE |
| 251 | 2.40E−08 | 0.39347767 | 0.00059037 | Monocyte | LINC01970 | TRUE | FALSE |
| 252 | 2.43E−08 | 0.57774032 | 0.0005985 | Monocyte | LAX1 | TRUE | FALSE |
| 253 | 2.44E−08 | 0.44979743 | 0.00060147 | Monocyte | SLC25A33 | TRUE | FALSE |
| 254 | 2.77E−08 | 0.21759122 | 0.00068228 | Monocyte | AC092718.1 | TRUE | FALSE |
| 255 | 2.78E−08 | 0.32450957 | 0.00068314 | Monocyte | AL161421.1 | TRUE | FALSE |
| 256 | 3.01E−08 | 0.29688454 | 0.00074136 | Monocyte | AC073934.1 | TRUE | FALSE |
| 257 | 3.02E−08 | −0.4308178 | 0.00074433 | Monocyte | CLOCK | TRUE | FALSE |
| 258 | 3.26E−08 | 0.14642949 | 0.00080215 | Monocyte | Z98742.4 | TRUE | FALSE |
| 259 | 3.51E−08 | −0.3520742 | 0.00086393 | Monocyte | TMEM168 | TRUE | FALSE |
| 260 | 3.52E−08 | 0.48385803 | 0.00086604 | Monocyte | GZF1 | TRUE | FALSE |
| 261 | 3.62E−08 | 0.20821033 | 0.00089141 | Monocyte | AC092053.2 | TRUE | FALSE |
| 262 | 3.69E−08 | 1.29311534 | 0.00090901 | Monocyte | AL450992.3 | TRUE | TRUE |
| 263 | 4.03E−08 | 0.5966957 | 0.0009908 | Monocyte | THAP9 | TRUE | FALSE |
| 264 | 4.18E−08 | 0.28039992 | 0.00102943 | Monocyte | LINC01344 | TRUE | FALSE |
| 265 | 4.24E−08 | −0.4862456 | 0.00104429 | Monocyte | ZNF397 | TRUE | FALSE |
| 266 | 4.27E−08 | 0.4756668 | 0.0010501 | Monocyte | IFFO2 | TRUE | FALSE |
| 267 | 4.29E−08 | 0.10898835 | 0.00105447 | Monocyte | AC100835.1 | TRUE | FALSE |
| 268 | 4.41E−08 | 0.12020561 | 0.00108612 | Monocyte | CT70 | TRUE | FALSE |
| 269 | 4.53E−08 | 0.26165458 | 0.00111434 | Monocyte | AC098818.2 | TRUE | FALSE |
| 270 | 4.67E−08 | −0.3446443 | 0.00114851 | Monocyte | LNPEP | TRUE | FALSE |
| 271 | 4.75E−08 | −0.4279562 | 0.00116972 | Monocyte | TRIM56 | TRUE | FALSE |
| 272 | 4.85E−08 | 0.19718917 | 0.00119336 | Monocyte | LINC01554 | TRUE | FALSE |
| 273 | 5.22E−08 | −0.3821855 | 0.00128419 | Monocyte | ATF7 | TRUE | FALSE |
| 274 | 5.48E−08 | 0.3046362 | 0.0013484 | Monocyte | ERCC1 | TRUE | FALSE |
| 275 | 5.71E−08 | 0.43343039 | 0.00140606 | Monocyte | BRCA2 | TRUE | FALSE |
| 276 | 5.72E−08 | 0.20282443 | 0.0014076 | Monocyte | AL031727.2 | TRUE | FALSE |
| 277 | 5.77E−08 | −0.3854788 | 0.00141858 | Monocyte | DCAF10 | TRUE | FALSE |
| 278 | 5.87E−08 | −0.3618951 | 0.00144397 | Monocyte | AP000763.3 | TRUE | FALSE |
| 279 | 5.95E−08 | 0.2250934 | 0.00146301 | Monocyte | LINC02357 | TRUE | FALSE |
| 280 | 6.02E−08 | 0.14886338 | 0.00148248 | Monocyte | GTF2IRD1 | TRUE | FALSE |
| 281 | 6.12E−08 | −0.3363886 | 0.00150668 | Monocyte | PHC3 | TRUE | FALSE |
| 282 | 6.27E−08 | 0.23510521 | 0.00154318 | Monocyte | AC022868.2 | TRUE | FALSE |
| 283 | 6.67E−08 | −0.332875 | 0.00164099 | Monocyte | ASXL2 | TRUE | FALSE |
| 284 | 6.72E−08 | 0.2657536 | 0.00165281 | Monocyte | AC084871.3 | TRUE | FALSE |
| 285 | 6.98E−08 | 0.24358377 | 0.00171661 | Monocyte | AC022075.1 | TRUE | FALSE |
| 286 | 7.05E−08 | −0.3897825 | 0.0017343 | Monocyte | MFSD4B | TRUE | FALSE |
| 287 | 7.13E−08 | 0.21583788 | 0.0017545 | Monocyte | PRRG2 | TRUE | FALSE |
| 288 | 7.23E−08 | −0.5775232 | 0.00177984 | Monocyte | AC007216.4 | TRUE | FALSE |
| 289 | 7.72E−08 | 0.14384147 | 0.00189901 | Monocyte | FBXO16 | TRUE | FALSE |
| 290 | 7.81E−08 | 0.37973445 | 0.00192279 | Monocyte | MBNL1-AS1 | TRUE | FALSE |
| 291 | 7.86E−08 | −0.2998697 | 0.00193301 | Monocyte | ZNF512 | TRUE | FALSE |
| 292 | 7.86E−08 | 0.19415708 | 0.00193341 | Monocyte | AC127002.2 | TRUE | FALSE |
| 293 | 8.26E−08 | 0.17062435 | 0.00203308 | Monocyte | AC099541.1 | TRUE | FALSE |
| 294 | 8.29E−08 | 0.37133586 | 0.00203897 | Monocyte | AC115618.1 | TRUE | FALSE |
| 295 | 8.39E−08 | 0.1761674 | 0.00206446 | Monocyte | PEBP4 | TRUE | FALSE |
| 296 | 8.56E−08 | 0.13951198 | 0.00210507 | Monocyte | AC087482.1 | TRUE | FALSE |
| 297 | 8.65E−08 | 0.14824092 | 0.0021276 | Monocyte | ULBP1 | TRUE | FALSE |
| 298 | 9.21E−08 | 0.2950386 | 0.00226724 | Monocyte | GPR137C | TRUE | FALSE |
| 299 | 9.99E−08 | 0.23141147 | 0.00245725 | Monocyte | GTF2F1 | TRUE | FALSE |
| 300 | 1.00E−07 | 0.19127861 | 0.00247143 | Monocyte | AL136038.3 | TRUE | FALSE |
| 301 | 1.06E−07 | 0.69126178 | 0.00260223 | Monocyte | TAGAP | TRUE | FALSE |
| 302 | 1.11E−07 | −0.2918835 | 0.00272053 | Monocyte | PANK3 | TRUE | FALSE |
| 303 | 1.11E−07 | −0.3619668 | 0.00272615 | Monocyte | PIP5K1A | TRUE | FALSE |
| 304 | 1.18E−07 | 0.14534494 | 0.00291445 | Monocyte | AL512288.1 | TRUE | FALSE |
| 305 | 1.21E−07 | −0.43031 | 0.00297246 | Monocyte | AKAP10 | TRUE | FALSE |
| 306 | 1.21E−07 | 0.11362667 | 0.00297814 | Monocyte | Z99572.1 | TRUE | FALSE |
| 307 | 1.22E−07 | −0.3044088 | 0.00300551 | Monocyte | C6orf62 | TRUE | FALSE |
| 308 | 1.23E−07 | −0.3590769 | 0.0030247 | Monocyte | RC3H2 | TRUE | FALSE |
| 309 | 1.24E−07 | −0.2322913 | 0.00304906 | Monocyte | FP236383.4 | TRUE | FALSE |
| 310 | 1.33E−07 | −0.3524626 | 0.00327657 | Monocyte | NAPEPLD | TRUE | FALSE |
| 311 | 1.35E−07 | 0.38581613 | 0.00333403 | Monocyte | IFRD1 | TRUE | FALSE |
| 312 | 1.37E−07 | −0.4170825 | 0.0033703 | Monocyte | ZFP14 | TRUE | FALSE |
| 313 | 1.37E−07 | 0.28790628 | 0.00338108 | Monocyte | CAHM | TRUE | FALSE |
| 314 | 1.63E−07 | −0.271883 | 0.00401197 | Monocyte | ZNF740 | TRUE | FALSE |
| 315 | 1.73E−07 | 0.39773964 | 0.00426749 | Monocyte | AC124016.1 | TRUE | FALSE |
| 316 | 1.82E−07 | 0.2405349 | 0.00448695 | Monocyte | LINC01185 | TRUE | FALSE |
| 317 | 1.83E−07 | −0.4186011 | 0.0045134 | Monocyte | NHLRC2 | TRUE | FALSE |
| 318 | 1.93E−07 | 0.18850719 | 0.00474427 | Monocyte | ZNF695 | TRUE | FALSE |
| 319 | 1.99E−07 | 0.3427572 | 0.00489257 | Monocyte | ZBTB10 | TRUE | FALSE |
| 320 | 2.00E−07 | −0.437714 | 0.00491167 | Monocyte | CCDC18-AS1 | TRUE | FALSE |
| 321 | 2.01E−07 | 0.57342706 | 0.00493352 | Monocyte | CDHR2 | TRUE | FALSE |
| 322 | 2.01E−07 | 0.47574236 | 0.00493883 | Monocyte | AP000943.2 | TRUE | FALSE |
| 323 | 2.09E−07 | −0.1973842 | 0.00513303 | Monocyte | ZNF700 | TRUE | FALSE |
| 324 | 2.15E−07 | 0.15250804 | 0.00528967 | Monocyte | GEM | TRUE | FALSE |
| 325 | 2.16E−07 | 0.15517853 | 0.00530594 | Monocyte | AP003680.1 | TRUE | FALSE |
| 326 | 2.21E−07 | 0.40798681 | 0.00543831 | Monocyte | AL645728.1 | TRUE | FALSE |
| 327 | 2.23E−07 | 0.49325109 | 0.00548758 | Monocyte | HIST2H2AC | TRUE | FALSE |
| 328 | 2.25E−07 | 0.1490935 | 0.00554371 | Monocyte | GORAB-AS1 | TRUE | FALSE |
| 329 | 2.25E−07 | −0.4248718 | 0.00554387 | Monocyte | TMEM161B- | TRUE | FALSE |
| | | | | | AS1 | | |
| 330 | 2.26E−07 | −0.3553391 | 0.00556202 | Monocyte | MFAP3 | TRUE | FALSE |
| 331 | 2.27E−07 | −0.3896879 | 0.00558153 | Monocyte | AC005261.1 | TRUE | FALSE |
| 332 | 2.27E−07 | 0.24319351 | 0.00559196 | Monocyte | HNRNPA0 | TRUE | FALSE |
| 333 | 2.27E−07 | 0.13975213 | 0.00559635 | Monocyte | AC024940.1 | TRUE | FALSE |
| 334 | 2.52E−07 | −0.3055641 | 0.00619028 | Monocyte | RASA1 | TRUE | FALSE |
| 335 | 2.60E−07 | 0.41326867 | 0.0063907 | Monocyte | CRY2 | TRUE | FALSE |
| 336 | 2.78E−07 | 0.3175925 | 0.00683574 | Monocyte | STX17-AS1 | TRUE | FALSE |
| 337 | 2.80E−07 | 0.15143823 | 0.00687783 | Monocyte | GLTPD2 | TRUE | FALSE |
| 338 | 2.84E−07 | 0.13471902 | 0.00698948 | Monocyte | LINC00471 | TRUE | FALSE |
| 339 | 2.90E−07 | 0.13627371 | 0.00712596 | Monocyte | ARMC5 | TRUE | FALSE |
| 340 | 2.90E−07 | −0.2500012 | 0.00714456 | Monocyte | EXOC1 | TRUE | FALSE |
| 341 | 2.93E−07 | 0.51920232 | 0.00719997 | Monocyte | KLF6 | TRUE | FALSE |
| 342 | 3.08E−07 | 0.26228987 | 0.00757302 | Monocyte | AC079807.1 | TRUE | FALSE |
| 343 | 3.09E−07 | 0.13354069 | 0.00760222 | Monocyte | AP000845.1 | TRUE | FALSE |
| 344 | 3.14E−07 | −0.3039075 | 0.00772918 | Monocyte | SEC22A | TRUE | FALSE |
| 345 | 3.27E−07 | 0.85420793 | 0.00805722 | Monocyte | AC044849.1 | TRUE | FALSE |
| 346 | 3.32E−07 | 0.27732504 | 0.00816866 | Monocyte | AL121603.2 | TRUE | FALSE |
| 347 | 3.35E−07 | −0.2069704 | 0.00825418 | Monocyte | FP671120.7 | TRUE | FALSE |
| 348 | 3.40E−07 | 0.15208847 | 0.00836733 | Monocyte | AC104078.2 | TRUE | FALSE |
| 349 | 3.67E−07 | −0.2879444 | 0.0090285 | Monocyte | WASHC4 | TRUE | FALSE |
| 350 | 3.69E−07 | 0.20798168 | 0.00906946 | Monocyte | AC009053.2 | TRUE | FALSE |
| 351 | 4.06E−07 | 0.10338668 | 0.0099922 | Monocyte | AC087241.2 | TRUE | FALSE |
| 352 | 4.22E−07 | 0.14567712 | 0.01038229 | Monocyte | SF3B2 | TRUE | FALSE |
| 353 | 4.51E−07 | −0.2808251 | 0.01109736 | Monocyte | TBL1XR1 | TRUE | FALSE |
| 354 | 4.51E−07 | 0.27412371 | 0.01110473 | Monocyte | AC023157.3 | TRUE | FALSE |
| 355 | 4.57E−07 | 0.43128041 | 0.01124564 | Monocyte | AC025164.1 | TRUE | FALSE |
| 356 | 4.63E−07 | 0.26917942 | 0.01139841 | Monocyte | AP000919.3 | TRUE | FALSE |
| 357 | 4.84E−07 | −0.5792826 | 0.01190074 | Monocyte | HMGA1P4 | TRUE | FALSE |
| 358 | 4.87E−07 | 0.72326135 | 0.01198492 | Monocyte | AC087239.1 | TRUE | FALSE |
| 359 | 4.90E−07 | −0.7899588 | 0.01206303 | Monocyte | AL034397.3 | TRUE | FALSE |
| 360 | 4.97E−07 | 0.27952691 | 0.01223199 | Monocyte | USP36 | TRUE | FALSE |
| 361 | 4.99E−07 | 0.16690332 | 0.01228859 | Monocyte | LINC01412 | TRUE | FALSE |
| 362 | 5.21E−07 | 0.22616887 | 0.01282434 | Monocyte | RABIF | TRUE | FALSE |
| 363 | 5.27E−07 | 0.34772732 | 0.0129591 | Monocyte | NCBP2AS2 | TRUE | FALSE |
| 364 | 5.30E−07 | −0.2964874 | 0.01303851 | Monocyte | HDAC8 | TRUE | FALSE |
| 365 | 5.37E−07 | 0.1368189 | 0.01321603 | Monocyte | LINC00677 | TRUE | FALSE |
| 366 | 5.52E−07 | 0.28940954 | 0.0135707 | Monocyte | RNF139 | TRUE | FALSE |
| 367 | 5.82E−07 | 0.1670188 | 0.01430865 | Monocyte | PRR3 | TRUE | FALSE |
| 368 | 5.86E−07 | 0.31386286 | 0.01442031 | Monocyte | AP001437.2 | TRUE | FALSE |
| 369 | 5.87E−07 | 0.14163633 | 0.01445387 | Monocyte | RHCE | TRUE | FALSE |
| 370 | 6.06E−07 | 0.24735521 | 0.01490313 | Monocyte | GINS4 | TRUE | FALSE |
| 371 | 6.12E−07 | 0.14309583 | 0.01506094 | Monocyte | DSEL | TRUE | FALSE |
| 372 | 6.36E−07 | 1.25795029 | 0.01564551 | Monocyte | FOSB | TRUE | TRUE |
| 373 | 6.37E−07 | 0.16258282 | 0.01567682 | Monocyte | CAGE1 | TRUE | FALSE |
| 374 | 6.47E−07 | 0.13489084 | 0.01591643 | Monocyte | AC117394.2 | TRUE | FALSE |
| 375 | 6.50E−07 | −0.3718947 | 0.01599916 | Monocyte | ZNF234 | TRUE | FALSE |
| 376 | 6.54E−07 | 0.11288849 | 0.01609785 | Monocyte | AC026202.3 | TRUE | FALSE |
| 377 | 6.66E−07 | 0.37890128 | 0.0163997 | Monocyte | AL390957.1 | TRUE | FALSE |
| 378 | 6.81E−07 | 0.40211313 | 0.01675464 | Monocyte | AC139099.2 | TRUE | FALSE |
| 379 | 7.12E−07 | 0.16343897 | 0.01752242 | Monocyte | AL035411.3 | TRUE | FALSE |
| 380 | 7.15E−07 | 0.11613791 | 0.0175874 | Monocyte | AC006449.2 | TRUE | FALSE |
| 381 | 7.24E−07 | 0.59549375 | 0.01782256 | Monocyte | LINC00309 | TRUE | FALSE |
| 382 | 7.24E−07 | 0.51815129 | 0.01782678 | Monocyte | AP001269.4 | TRUE | FALSE |
| 383 | 7.32E−07 | 0.50445264 | 0.01800549 | Monocyte | AC007384.1 | TRUE | FALSE |
| 384 | 7.35E−07 | 0.36377255 | 0.01807526 | Monocyte | PLK2 | TRUE | FALSE |
| 385 | 7.53E−07 | 0.1254361 | 0.01851716 | Monocyte | USP2 | TRUE | FALSE |
| 386 | 7.67E−07 | 0.23413537 | 0.01886555 | Monocyte | LPP-AS2 | TRUE | FALSE |
| 387 | 7.72E−07 | −0.3854904 | 0.01898927 | Monocyte | LINC01355 | TRUE | FALSE |
| 388 | 7.73E−07 | 0.26224168 | 0.01902641 | Monocyte | PTS | TRUE | FALSE |
| 389 | 7.79E−07 | 0.07067923 | 0.01916928 | Monocyte | AC012640.1 | TRUE | FALSE |
| 390 | 7.96E−07 | 0.28713367 | 0.01958747 | Monocyte | GABPB1-IT1 | TRUE | FALSE |
| 391 | 8.18E−07 | 0.35926824 | 0.02013762 | Monocyte | ADPGK-AS1 | TRUE | FALSE |
| 392 | 8.24E−07 | 0.18905621 | 0.02028413 | Monocyte | SPAG4 | TRUE | FALSE |
| 393 | 8.25E−07 | 0.26517189 | 0.02030231 | Monocyte | AL158071.3 | TRUE | FALSE |
| 394 | 8.29E−07 | −0.2420561 | 0.0204014 | Monocyte | APC | TRUE | FALSE |
| 395 | 8.67E−07 | 0.10095894 | 0.0213276 | Monocyte | CEP83-DT | TRUE | FALSE |
| 396 | 8.70E−07 | 0.14947397 | 0.02141077 | Monocyte | HNRNPU | TRUE | FALSE |
| 397 | 8.95E−07 | 0.22598869 | 0.02202748 | Monocyte | ZMIZ1-AS1 | TRUE | FALSE |
| 398 | 9.23E−07 | 0.11460511 | 0.02270881 | Monocyte | AC009292.2 | TRUE | FALSE |
| 399 | 9.71E−07 | 0.53383309 | 0.02389825 | Monocyte | ZFAND2A | TRUE | FALSE |
| 400 | 1.02E−06 | 0.11890951 | 0.02518627 | Monocyte | AC007881.3 | TRUE | FALSE |
| 401 | 1.06E−06 | −0.410546 | 0.02606518 | Monocyte | CSTF3 | TRUE | FALSE |
| 402 | 1.10E−06 | −0.2900855 | 0.026965 | Monocyte | RNF170 | TRUE | FALSE |
| 403 | 1.10E−06 | −0.2158548 | 0.02709905 | Monocyte | KDM5A | TRUE | FALSE |
| 404 | 1.12E−06 | −0.2829467 | 0.02762326 | Monocyte | LPGAT1 | TRUE | FALSE |
| 405 | 1.14E−06 | −0.3011287 | 0.02804626 | Monocyte | GPATCH2L | TRUE | FALSE |
| 406 | 1.15E−06 | 1.11198242 | 0.02818609 | Monocyte | Z93241.1 | TRUE | TRUE |
| 407 | 1.16E−06 | 0.23062852 | 0.02845183 | Monocyte | CDC37L1-DT | TRUE | FALSE |
| 408 | 1.19E−06 | 0.11713364 | 0.02921839 | Monocyte | LINC02292 | TRUE | FALSE |
| 409 | 1.20E−06 | 0.3028575 | 0.02960895 | Monocyte | DTHD1 | TRUE | FALSE |
| 410 | 1.21E−06 | 0.31375501 | 0.02968591 | Monocyte | AC004917.1 | TRUE | FALSE |
| 411 | 1.21E−06 | −0.2750701 | 0.02972437 | Monocyte | RABGAP1 | TRUE | FALSE |
| 412 | 1.22E−06 | −0.3863684 | 0.02992409 | Monocyte | ZNF75D | TRUE | FALSE |
| 413 | 1.22E−06 | 0.12000848 | 0.03012282 | Monocyte | AL121761.1 | TRUE | FALSE |
| 414 | 1.23E−06 | 0.41718376 | 0.0302229 | Monocyte | SMPDL3B | TRUE | FALSE |
| 415 | 1.27E−06 | −0.2354232 | 0.03126359 | Monocyte | PPP1R21 | TRUE | FALSE |
| 416 | 1.28E−06 | 0.11941138 | 0.03148502 | Monocyte | AC083843.2 | TRUE | FALSE |
| 417 | 1.29E−06 | 0.10242718 | 0.03162328 | Monocyte | AC107398.5 | TRUE | FALSE |
| 418 | 1.29E−06 | 0.16084054 | 0.03184933 | Monocyte | AL137003.1 | TRUE | FALSE |
| 419 | 1.32E−06 | 0.23865025 | 0.03243429 | Monocyte | DNAAF2 | TRUE | FALSE |
| 420 | 1.34E−06 | −0.203822 | 0.03288901 | Monocyte | IREB2 | TRUE | FALSE |
| 421 | 1.35E−06 | −0.4284832 | 0.03314392 | Monocyte | AC025682.1 | TRUE | FALSE |
| 422 | 1.38E−06 | 0.38495187 | 0.03401362 | Monocyte | SESN2 | TRUE | FALSE |
| 423 | 1.40E−06 | 0.16517318 | 0.03438714 | Monocyte | AL031848.2 | TRUE | FALSE |
| 424 | 1.42E−06 | 0.50328675 | 0.03505461 | Monocyte | PHACTR1 | TRUE | FALSE |
| 425 | 1.43E−06 | −0.3856837 | 0.03512196 | Monocyte | CBR4 | TRUE | FALSE |
| 426 | 1.47E−06 | 0.24971052 | 0.03608739 | Monocyte | NFYC-AS1 | TRUE | FALSE |
| 427 | 1.48E−06 | −0.4223332 | 0.03632684 | Monocyte | ZNF81 | TRUE | FALSE |
| 428 | 1.48E−06 | 0.13264243 | 0.03638062 | Monocyte | RNPS1 | TRUE | FALSE |
| 429 | 1.52E−06 | 0.60494712 | 0.03737802 | Monocyte | C4orf47 | TRUE | FALSE |
| 430 | 1.54E−06 | −0.2566814 | 0.03800183 | Monocyte | HIF1AN | TRUE | FALSE |
| 431 | 1.57E−06 | 0.50345801 | 0.03868756 | Monocyte | FAM161B | TRUE | FALSE |
| 432 | 1.59E−06 | 0.25831857 | 0.03906923 | Monocyte | SF3A1 | TRUE | FALSE |
| 433 | 1.63E−06 | 0.38098887 | 0.0400603 | Monocyte | CLK1 | TRUE | FALSE |
| 434 | 1.64E−06 | −0.3353589 | 0.04030679 | Monocyte | DDI2 | TRUE | FALSE |
| 435 | 1.65E−06 | 0.34983595 | 0.04047851 | Monocyte | ZBTB24 | TRUE | FALSE |
| 436 | 1.66E−06 | 0.34408606 | 0.04083511 | Monocyte | TRA2B | TRUE | FALSE |
| 437 | 1.72E−06 | 0.35855987 | 0.04220957 | Monocyte | MEX3C | TRUE | FALSE |
| 438 | 1.72E−06 | 0.14479585 | 0.04223422 | Monocyte | U91328.2 | TRUE | FALSE |
| 439 | 1.72E−06 | 0.36150724 | 0.04242194 | Monocyte | ARID5A | TRUE | FALSE |
| 440 | 1.75E−06 | 0.19626852 | 0.04295787 | Monocyte | MATR3.1 | TRUE | FALSE |
| 441 | 1.76E−06 | 0.45507572 | 0.04326185 | Monocyte | PRR7 | TRUE | FALSE |
| 442 | 1.77E−06 | 0.22162575 | 0.04348724 | Monocyte | EIF2AK3-DT | TRUE | FALSE |
| 443 | 1.80E−06 | −0.2045698 | 0.04434091 | Monocyte | DPP8 | TRUE | FALSE |
| 444 | 1.83E−06 | 0.61385081 | 0.04495616 | Monocyte | CSRNP1 | TRUE | FALSE |
| 445 | 1.84E−06 | −0.4281948 | 0.04517349 | Monocyte | ZNF710 | TRUE | FALSE |
| 446 | 1.87E−06 | 0.45979184 | 0.04611505 | Monocyte | KMT2E-AS1 | TRUE | FALSE |
| 447 | 1.88E−06 | 0.48053168 | 0.04631614 | Monocyte | AL158152.1 | TRUE | FALSE |
| 448 | 1.91E−06 | 0.11422649 | 0.04708678 | Monocyte | AL022328.3 | TRUE | FALSE |
| 449 | 1.93E−06 | 0.19387495 | 0.04737651 | Monocyte | PMEL | TRUE | FALSE |
| 450 | 1.99E−06 | 0.49212989 | 0.04894703 | Monocyte | RRP12 | TRUE | FALSE |
| 451 | 2.00E−06 | 0.43040876 | 0.0491872 | Monocyte | C6orf99 | TRUE | FALSE |
| 452 | 4.95E−22 | 1.98241839 | 1.22E−17 | B.cell | AC007952.4 | TRUE | TRUE |
| 453 | 7.74E−17 | 1.37187398 | 1.90E−12 | B.cell | Z93241.1 | TRUE | TRUE |
| 454 | 1.37E−15 | 1.81898366 | 3.36E−11 | B.cell | AC245014.3 | TRUE | TRUE |
| 455 | 2.19E−14 | 1.7623009 | 5.38E−10 | B.cell | TEX14 | TRUE | TRUE |
| 456 | 8.74E−14 | 1.16720284 | 2.15E−09 | B.cell | AL021155.5 | TRUE | TRUE |
| 457 | 1.65E−11 | 1.10631588 | 4.05E−07 | B.cell | NR4A2 | TRUE | TRUE |
| 458 | 1.75E−11 | 1.72955417 | 4.32E−07 | B.cell | AC253572.2 | TRUE | TRUE |
| 459 | 1.47E−09 | 0.99412601 | 3.61E−05 | B.cell | AC012447.1 | TRUE | FALSE |
| 460 | 1.51E−09 | 0.97653112 | 3.72E−05 | B.cell | AC022217.3 | TRUE | FALSE |
| 461 | 3.79E−09 | 1.53303082 | 9.32E−05 | B.cell | FOS | TRUE | TRUE |
| 462 | 5.03E−09 | 0.67958753 | 0.00012384 | B.cell | MTMR6 | TRUE | FALSE |
| 463 | 1.14E−08 | 0.47688946 | 0.00028134 | B.cell | YPEL5 | TRUE | FALSE |
| 464 | 3.54E−08 | 0.46796316 | 0.00087174 | B.cell | IQGAP1 | TRUE | FALSE |
| 465 | 9.75E−08 | 2.46673669 | 0.00239901 | B.cell | IGHV4-34 | TRUE | TRUE |
| 466 | 1.01E−07 | 0.98149255 | 0.00249108 | B.cell | JUNB | TRUE | FALSE |
| 467 | 1.01E−07 | 0.31759615 | 0.00249291 | B.cell | SLC38A2 | TRUE | FALSE |
| 468 | 1.22E−07 | 0.73180378 | 0.00299417 | B.cell | SIAH2-AS1 | TRUE | FALSE |
| 469 | 1.23E−07 | 0.54210427 | 0.00302352 | B.cell | WDR74 | TRUE | FALSE |
| 470 | 1.43E−07 | 1.29645999 | 0.00350868 | B.cell | AC044849.1 | TRUE | TRUE |
| 471 | 1.46E−07 | 0.81090532 | 0.00359437 | B.cell | LINC00910 | TRUE | FALSE |
| 472 | 1.50E−07 | 0.66177742 | 0.00368322 | B.cell | AL499604.1 | TRUE | FALSE |
| 473 | 1.78E−07 | 0.6765215 | 0.00437127 | B.cell | AC091271.1 | TRUE | FALSE |
| 474 | 2.62E−07 | 0.64916014 | 0.00643891 | B.cell | COQ7 | TRUE | FALSE |
| 475 | 3.22E−07 | 1.19475525 | 0.00791356 | B.cell | DUSP1 | TRUE | TRUE |
| 476 | 3.49E−07 | 0.73011976 | 0.00859873 | B.cell | C9orf72 | TRUE | FALSE |
| 477 | 3.97E−07 | 0.55658796 | 0.00976486 | B.cell | DBF4 | TRUE | FALSE |
| 478 | 4.01E−07 | 1.35157089 | 0.00987656 | B.cell | FOSB | TRUE | TRUE |
| 479 | 5.04E−07 | 0.90396609 | 0.0123952 | B.cell | AC103591.3 | TRUE | FALSE |
| 480 | 6.56E−07 | 0.5872996 | 0.01614692 | B.cell | NFKBIZ | TRUE | FALSE |
| 481 | 7.82E−07 | 0.39474088 | 0.01924701 | B.cell | CROCC | TRUE | FALSE |
| 482 | 1.09E−06 | 0.55421231 | 0.02686252 | B.cell | EPS8 | TRUE | FALSE |
| 483 | 1.64E−06 | 0.76728684 | 0.04045725 | B.cell | HIST1H2BG | TRUE | FALSE |
| 484 | 1.74E−06 | 0.44589874 | 0.04275311 | B.cell | RANBP2 | TRUE | FALSE |
| 485 | 1.74E−06 | 1.12962302 | 0.04292917 | B.cell | BFSP2 | TRUE | TRUE |
| 486 | 1.83E−35 | 1.37763071 | 4.49E−31 | T.cell | AC245014.3 | TRUE | TRUE |
| 487 | 1.38E−33 | 1.4258843 | 3.40E−29 | T.cell | AC007952.4 | TRUE | TRUE |
| 488 | 1.23E−23 | 1.49357265 | 3.03E−19 | T.cell | TEX14 | TRUE | TRUE |
| 489 | 4.08E−19 | 1.3663231 | 1.00E−14 | T.cell | LINC00910 | TRUE | TRUE |
| 490 | 1.27E−15 | 0.99656227 | 3.12E−11 | T.cell | Z93241.1 | TRUE | FALSE |
| 491 | 4.80E−15 | 0.57744448 | 1.18E−10 | T.cell | AC083880.1 | TRUE | FALSE |
| 492 | 7.60E−14 | 0.78236071 | 1.87E−09 | T.cell | AL021155.5 | TRUE | FALSE |
| 493 | 2.72E−12 | 0.61322337 | 6.69E−08 | T.cell | Z99127.4 | TRUE | FALSE |
| 494 | 7.45E−12 | 0.67117702 | 1.83E−07 | T.cell | HIST1H3A | TRUE | FALSE |
| 495 | 1.12E−11 | 0.78051511 | 2.77E−07 | T.cell | AL499604.1 | TRUE | FALSE |
| 496 | 1.92E−11 | 0.57278858 | 4.73E−07 | T.cell | AC104695.2 | TRUE | FALSE |
| 497 | 2.58E−11 | 0.68526307 | 6.34E−07 | T.cell | SIAH2-AS1 | TRUE | FALSE |
| 498 | 6.17E−11 | 0.60200475 | 1.52E−06 | T.cell | EFCAB2 | TRUE | FALSE |
| 499 | 2.79E−10 | 1.34301342 | 6.86E−06 | T.cell | AC253572.2 | TRUE | TRUE |
| 500 | 1.19E−09 | 1.28518974 | 2.94E−05 | T.cell | JUN | TRUE | TRUE |
| 501 | 2.16E−09 | 0.57785765 | 5.31E−05 | T.cell | AC012447.1 | TRUE | FALSE |
| 502 | 2.28E−09 | −0.5184248 | 5.61E−05 | T.cell | LINC00861 | TRUE | FALSE |
| 503 | 3.03E−09 | 0.49137188 | 7.46E−05 | T.cell | AL137779.2 | TRUE | FALSE |
| 504 | 3.31E−09 | 0.95676718 | 8.14E−05 | T.cell | DUSP1 | TRUE | FALSE |
| 505 | 5.04E−09 | 0.4186882 | 0.00012409 | T.cell | TERC | TRUE | FALSE |
| 506 | 5.07E−09 | 0.48839554 | 0.00012483 | T.cell | AL645728.1 | TRUE | FALSE |
| 507 | 5.15E−09 | 0.47903877 | 0.0001266 | T.cell | SREBF2-AS1 | TRUE | FALSE |
| 508 | 5.15E−09 | 0.44865169 | 0.0001268 | T.cell | HIST1H2BG | TRUE | FALSE |
| 509 | 5.77E−09 | 0.41305186 | 0.00014206 | T.cell | PTCH2 | TRUE | FALSE |
| 510 | 6.81E−09 | 0.50877589 | 0.0001675 | T.cell | AP003717.4 | TRUE | FALSE |
| 511 | 1.18E−08 | 0.6631811 | 0.00029107 | T.cell | AC239799.2 | TRUE | FALSE |
| 512 | 1.72E−08 | 0.57928721 | 0.0004242 | T.cell | AF111167.1 | TRUE | FALSE |
| 513 | 1.93E−08 | 0.50626064 | 0.0004744 | T.cell | AC103591.3 | TRUE | FALSE |
| 514 | 2.89E−08 | 0.73467622 | 0.00071085 | T.cell | AC022217.3 | TRUE | FALSE |
| 515 | 4.43E−08 | 1.21208544 | 0.00108946 | T.cell | FOS | TRUE | TRUE |
| 516 | 4.99E−08 | 0.82631547 | 0.00122679 | T.cell | AC044849.1 | TRUE | FALSE |
| 517 | 5.43E−08 | 0.31005815 | 0.00133516 | T.cell | CCNL1 | TRUE | FALSE |
| 518 | 5.61E−08 | 1.14490316 | 0.00138136 | T.cell | FOSB | TRUE | TRUE |
| 519 | 6.32E−08 | 0.38670799 | 0.00155421 | T.cell | SLC38A2 | TRUE | FALSE |
| 520 | 7.76E−08 | 0.28566682 | 0.0019101 | T.cell | AC072061.1 | TRUE | FALSE |
| 521 | 1.05E−07 | 0.75245718 | 0.00257898 | T.cell | AL691403.1 | TRUE | FALSE |
| 522 | 1.26E−07 | 0.44831473 | 0.00310517 | T.cell | AC087239.1 | TRUE | FALSE |
| 523 | 1.33E−07 | 0.68178192 | 0.00327132 | T.cell | PMAIP1 | TRUE | FALSE |
| 524 | 1.71E−07 | 0.62768618 | 0.00421477 | T.cell | HIST1H2BN | TRUE | FALSE |
| 525 | 1.72E−07 | 0.28448895 | 0.00422581 | T.cell | AL353708.1 | TRUE | FALSE |
| 526 | 2.27E−07 | 0.43584412 | 0.00557561 | T.cell | KLF6 | TRUE | FALSE |
| 527 | 3.38E−07 | 0.35673045 | 0.00830513 | T.cell | ARRDC3-AS1 | TRUE | FALSE |
| 528 | 4.13E−07 | 0.42808453 | 0.01015289 | T.cell | EPS8 | TRUE | FALSE |
| 529 | 4.96E−07 | −0.4563722 | 0.01220621 | T.cell | ZNF780B | TRUE | FALSE |
| 530 | 5.23E−07 | 0.45548478 | 0.01287309 | T.cell | SCN11A | TRUE | FALSE |
| 531 | 5.46E−07 | 0.72944242 | 0.01342734 | T.cell | TNFAIP3 | TRUE | FALSE |
| 532 | 6.03E−07 | 0.47245061 | 0.01484553 | T.cell | AL163973.2 | TRUE | FALSE |
| 533 | 6.40E−07 | 0.30061177 | 0.01574781 | T.cell | AC103724.3 | TRUE | FALSE |
| 534 | 6.71E−07 | 0.48352245 | 0.0165196 | T.cell | ATP2B1-AS1 | TRUE | FALSE |
| 535 | 6.78E−07 | 0.42972151 | 0.01669474 | T.cell | AC091271.1 | TRUE | FALSE |
| 536 | 9.68E−07 | 0.32844881 | 0.0238257 | T.cell | AC020765.2 | TRUE | FALSE |
| 537 | 9.73E−07 | 0.36255848 | 0.02393057 | T.cell | RASA3 | TRUE | FALSE |
| 538 | 1.03E−06 | 0.30780405 | 0.02529169 | T.cell | AC013400.1 | TRUE | FALSE |
| 539 | 1.23E−06 | 0.31522374 | 0.03032022 | T.cell | AL109767.1 | TRUE | FALSE |
| 540 | 1.38E−06 | 0.75612758 | 0.0339332 | T.cell | JUNB | TRUE | FALSE |
| 541 | 1.71E−06 | −0.4070742 | 0.0421234 | T.cell | THUMPD3-AS1 | TRUE | FALSE |
| 542 | 1.83E−06 | −0.4015701 | 0.04507771 | T.cell | ZNF691 | TRUE | FALSE |
| 543 | 1.94E−06 | 0.42259434 | 0.04771898 | T.cell | C6orf99 | TRUE | FALSE |
| 544 | 6.09E−12 | 1.63547619 | 1.50E−07 | DC | JUN | TRUE | TRUE |
| 545 | 7.96E−11 | 1.9233684 | 1.96E−06 | DC | TEX14 | TRUE | TRUE |
| 546 | 3.63E−10 | 1.54842231 | 8.94E−06 | DC | Z93241.1 | TRUE | TRUE |
| 547 | 6.38E−10 | 1.7988273 | 1.57E−05 | DC | AC007952.4 | TRUE | TRUE |
| 548 | 1.10E−09 | 1.88064553 | 2.71E−05 | DC | AC245014.3 | TRUE | TRUE |
| 549 | 1.08E−08 | 2.16292045 | 0.00026616 | DC | AC253572.2 | TRUE | TRUE |
| 550 | 2.86E−07 | 1.51667074 | 0.00704025 | DC | LINC00910 | TRUE | TRUE |
| 551 | 8.53E−07 | 0.64260923 | 0.02098384 | DC | C9orf72 | TRUE | FALSE |
| 552 | 1.19E−06 | 1.24478063 | 0.02920493 | DC | TENT5C | TRUE | TRUE |
| 553 | 1.65E−06 | 1.5287835 | 0.04063363 | DC | AC103591.3 | TRUE | TRUE |
| 554 | 1.92E−06 | 1.05573041 | 0.04727022 | DC | AC022217.3 | TRUE | TRUE |
| 555 | 3.67E−22 | 1.43781945 | 9.04E−18 | Natural.killer | AC007952.4 | TRUE | TRUE |
| 556 | 3.42E−21 | 1.41383933 | 8.41E−17 | Natural.killer | LINC00910 | TRUE | TRUE |
| 557 | 3.82E−20 | 1.5235539 | 9.39E−16 | Natural.killer | AC245014.3 | TRUE | TRUE |
| 558 | 1.42E−18 | 1.71538998 | 3.48E−14 | Natural.killer | TEX14 | TRUE | TRUE |
| 559 | 2.52E−16 | 1.18728209 | 6.19E−12 | Natural.killer | Z93241.1 | TRUE | TRUE |
| 560 | 4.45E−16 | 1.2330183 | 1.09E−11 | Natural.killer | AC022217.3 | TRUE | TRUE |
| 561 | 3.92E−15 | 0.34393481 | 9.64E−11 | Natural.killer | ATP2B1-AS1 | TRUE | FALSE |
| 562 | 2.31E−14 | 1.02208619 | 5.67E−10 | Natural.killer | AL021155.5 | TRUE | TRUE |
| 563 | 2.19E−13 | 0.7695756 | 5.40E−09 | Natural.killer | AC091271.1 | TRUE | FALSE |
| 564 | 1.22E−12 | 1.47006157 | 3.00E−08 | Natural.killer | JUN | TRUE | TRUE |
| 565 | 1.58E−12 | 0.94606833 | 3.88E−08 | Natural.killer | AL499604.1 | TRUE | FALSE |
| 566 | 7.87E−12 | 0.66790399 | 1.94E−07 | Natural.killer | Z99127.4 | TRUE | FALSE |
| 567 | 8.97E−12 | 1.42558506 | 2.21E−07 | Natural.killer | FOSB | TRUE | TRUE |
| 568 | 1.42E−10 | 0.74213043 | 3.50E−06 | Natural.killer | HIST1H2BN | TRUE | FALSE |
| 569 | 3.58E−09 | 0.77850896 | 8.82E−05 | Natural.killer | AL513303.1 | TRUE | FALSE |
| 570 | 4.34E−09 | 0.80614641 | 0.00010676 | Natural.killer | SIAH2-AS1 | TRUE | FALSE |
| 571 | 9.44E−09 | 1.50202973 | 0.00023227 | Natural.killer | AC253572.2 | TRUE | TRUE |
| 572 | 3.03E−08 | 0.62235091 | 0.00074464 | Natural.killer | EFCAB2 | TRUE | FALSE |
| 573 | 3.69E−08 | 0.9078976 | 0.00090743 | Natural.killer | AP003717.4 | TRUE | FALSE |
| 574 | 6.81E−08 | 0.89185275 | 0.00167602 | Natural.killer | DUSP1 | TRUE | FALSE |
| 575 | 1.13E−07 | 0.47043375 | 0.00277278 | Natural.killer | WDR74 | TRUE | FALSE |
| 576 | 1.22E−07 | 0.64221227 | 0.00301234 | Natural.killer | EPS8 | TRUE | FALSE |
| 577 | 1.27E−07 | 0.84516023 | 0.00313157 | Natural.killer | AL691403.1 | TRUE | FALSE |
| 578 | 1.46E−07 | 0.88403202 | 0.00358058 | Natural.killer | HIST1H2BC | TRUE | FALSE |
| 579 | 2.78E−07 | 1.02003224 | 0.00684523 | Natural.killer | AC044849.1 | TRUE | TRUE |
| 580 | 3.57E−07 | 0.40565653 | 0.00879059 | Natural.killer | IER2 | TRUE | FALSE |
| 581 | 4.22E−07 | 0.86050939 | 0.01039027 | Natural.killer | HIST1H3A | TRUE | FALSE |
| 582 | 4.89E−07 | −0.3765739 | 0.01202514 | Natural.killer | TP53RK | TRUE | FALSE |
| 583 | 4.93E−07 | 0.74359687 | 0.012121 | Natural.killer | AC104695.2 | TRUE | FALSE |
| 584 | 5.10E−07 | 0.88988233 | 0.01254497 | Natural.killer | TSPYL2 | TRUE | FALSE |
| 585 | 5.56E−07 | 0.56574974 | 0.01366977 | Natural.killer | AC239799.2 | TRUE | FALSE |
| 586 | 5.99E−07 | 1.11122521 | 0.01474058 | Natural.killer | FOS | TRUE | TRUE |
| 587 | 9.82E−07 | 0.84036017 | 0.02416218 | Natural.killer | AC087239.1 | TRUE | FALSE |
| 588 | 1.01E−06 | 0.27163025 | 0.02493696 | Natural.killer | HIST1H3D | TRUE | FALSE |
| 589 | 1.05E−06 | 0.70567889 | 0.0257763 | Natural.killer | AF111167.1 | TRUE | FALSE |
| 590 | 1.36E−06 | 0.54126502 | 0.03335062 | Natural.killer | AC093510.1 | TRUE | FALSE |
| 591 | 1.48E−06 | 0.52710268 | 0.03641406 | Natural.killer | LINC01765 | TRUE | FALSE |
| 592 | 2.69E−32 | 0.51155437 | 6.62E−28 | all | MPP7-DT | TRUE | FALSE |
| 593 | 5.49E−30 | 0.48362122 | 1.35E−25 | all | AL137060.3 | TRUE | FALSE |
| 594 | 1.53E−24 | 0.95601065 | 3.77E−20 | all | AC104695.2 | TRUE | FALSE |
| 595 | 9.63E−24 | 1.07321771 | 2.37E−19 | all | MYOSLID | TRUE | TRUE |
| 596 | 2.92E−23 | 0.55089867 | 7.20E−19 | all | JARID2-AS1 | TRUE | FALSE |
| 597 | 4.70E−23 | 1.13457984 | 1.16E−18 | all | HLX-AS1 | TRUE | TRUE |
| 598 | 1.37E−22 | 0.62103103 | 3.37E−18 | all | AC023509.3 | TRUE | FALSE |
| 599 | 1.40E−22 | 0.41024461 | 3.44E−18 | all | EZR-AS1 | TRUE | FALSE |
| 600 | 2.98E−22 | 0.51876636 | 7.34E−18 | all | AL627171.1 | TRUE | FALSE |
| 601 | 5.27E−21 | 0.64539977 | 1.30E−16 | all | AL356512.1 | TRUE | FALSE |
| 602 | 2.05E−20 | 0.53631747 | 5.04E−16 | all | AC017083.1 | TRUE | FALSE |
| 603 | 2.07E−20 | 0.34558409 | 5.08E−16 | all | TERC | TRUE | FALSE |
| 604 | 5.54E−20 | 1.05025059 | 1.36E−15 | all | SIAH2-AS1 | TRUE | TRUE |
| 605 | 7.70E−20 | 0.45296922 | 1.90E−15 | all | AC069431.1 | TRUE | FALSE |
| 606 | 1.13E−19 | 0.29792932 | 2.78E−15 | all | AL512791.2 | TRUE | FALSE |
| 607 | 1.22E−19 | 1.22971961 | 3.01E−15 | all | ATP2B1-AS1 | TRUE | TRUE |
| 608 | 2.96E−19 | 0.58151732 | 7.28E−15 | all | AP003717.4 | TRUE | FALSE |
| 609 | 5.84E−19 | 0.53519565 | 1.44E−14 | all | HOOK2 | TRUE | FALSE |
| 610 | 8.34E−19 | 0.93527888 | 2.05E−14 | all | AC091271.1 | TRUE | FALSE |
| 611 | 1.10E−18 | 0.61631628 | 2.70E−14 | all | AC079305.1 | TRUE | FALSE |
| 612 | 1.34E−18 | 0.22407932 | 3.31E−14 | all | AL391832.4 | TRUE | FALSE |
| 613 | 1.83E−18 | 0.39705324 | 4.51E−14 | all | LINC02669 | TRUE | FALSE |
| 614 | 2.79E−18 | 0.93254708 | 6.87E−14 | all | AC008440.1 | TRUE | FALSE |
| 615 | 1.25E−17 | 0.49929671 | 3.08E−13 | all | AL450992.1 | TRUE | FALSE |
| 616 | 1.58E−17 | 0.46617266 | 3.90E−13 | all | AL359711.2 | TRUE | FALSE |
| 617 | 1.63E−17 | 0.44229344 | 4.02E−13 | all | AL353719.1 | TRUE | FALSE |
| 618 | 1.75E−17 | 1.07602704 | 4.32E−13 | all | AC022217.3 | TRUE | TRUE |
| 619 | 2.65E−17 | 0.445822 | 6.51E−13 | all | AC083880.1 | TRUE | FALSE |
| 620 | 3.11E−17 | 0.46908469 | 7.64E−13 | all | UBAC2-AS1 | TRUE | FALSE |
| 621 | 3.89E−17 | 0.2474219 | 9.57E−13 | all | AC007365.1 | TRUE | FALSE |
| 622 | 5.72E−17 | 0.83653469 | 1.41E−12 | all | AL158801.2 | TRUE | FALSE |
| 623 | 7.08E−17 | 0.30750153 | 1.74E−12 | all | AL121574.1 | TRUE | FALSE |
| 624 | 2.20E−16 | 0.31915934 | 5.42E−12 | all | AC006994.2 | TRUE | FALSE |
| 625 | 2.41E−16 | 0.71898013 | 5.94E−12 | all | SPAG5-AS1 | TRUE | FALSE |
| 626 | 2.88E−16 | 0.25572419 | 7.08E−12 | all | BX323046.1 | TRUE | FALSE |
| 627 | 3.14E−16 | 0.39891467 | 7.73E−12 | all | GNAT2 | TRUE | FALSE |
| 628 | 3.56E−16 | 0.51763376 | 8.75E−12 | all | AC110741.1 | TRUE | FALSE |
| 629 | 4.46E−16 | 0.46644226 | 1.10E−11 | all | AL139106.1 | TRUE | FALSE |
| 630 | 4.49E−16 | 0.98053011 | 1.11E−11 | all | NR4A2 | TRUE | FALSE |
| 631 | 7.30E−16 | 0.1656201 | 1.80E−11 | all | BX323046.2 | TRUE | FALSE |
| 632 | 1.58E−15 | 1.06495064 | 3.88E−11 | all | AC020911.2 | TRUE | TRUE |
| 633 | 2.11E−15 | 0.79250383 | 5.20E−11 | all | LINC01220 | TRUE | FALSE |
| 634 | 2.17E−15 | 0.17599181 | 5.34E−11 | all | LINC01126 | TRUE | FALSE |
| 635 | 3.15E−15 | 0.23990078 | 7.75E−11 | all | AL024507.2 | TRUE | FALSE |
| 636 | 3.95E−15 | 0.17471501 | 9.73E−11 | all | AC123777.1 | TRUE | FALSE |
| 637 | 6.40E−15 | 0.3709599 | 1.58E−10 | all | AC006511.6 | TRUE | FALSE |
| 638 | 7.86E−15 | 0.56487884 | 1.93E−10 | all | HIST1H2BN | TRUE | FALSE |
| 639 | 9.33E−15 | 0.58245117 | 2.30E−10 | all | BHLHE40-AS1 | TRUE | FALSE |
| 640 | 9.34E−15 | 0.20360576 | 2.30E−10 | all | AC112236.2 | TRUE | FALSE |
| 641 | 9.89E−15 | 0.65581034 | 2.43E−10 | all | COQ7 | TRUE | FALSE |
| 642 | 1.18E−14 | 0.76806129 | 2.91E−10 | all | AC007032.1 | TRUE | FALSE |
| 643 | 1.27E−14 | 0.25357275 | 3.13E−10 | all | AC091214.1 | TRUE | FALSE |
| 644 | 1.42E−14 | 0.44408247 | 3.50E−10 | all | AC010864.1 | TRUE | FALSE |
| 645 | 1.58E−14 | 0.80017679 | 3.89E−10 | all | EFCAB2 | TRUE | FALSE |
| 646 | 2.26E−14 | 0.26199429 | 5.57E−10 | all | GASAL1 | TRUE | FALSE |
| 647 | 2.34E−14 | 0.55701407 | 5.75E−10 | all | Z99127.4 | TRUE | FALSE |
| 648 | 2.46E−14 | 0.4257923 | 6.05E−10 | all | DNAJB5-DT | TRUE | FALSE |
| 649 | 3.01E−14 | 0.23122939 | 7.40E−10 | all | AC008115.1 | TRUE | FALSE |
| 650 | 6.28E−14 | 0.83827115 | 1.55E−09 | all | AL499604.1 | TRUE | FALSE |
| 651 | 6.32E−14 | 0.29129145 | 1.56E−09 | all | UBE2R2-AS1 | TRUE | FALSE |
| 652 | 7.72E−14 | 0.2404076 | 1.90E−09 | all | AL138895.1 | TRUE | FALSE |
| 653 | 1.01E−13 | 0.38639875 | 2.48E−09 | all | HIST1H2BG | TRUE | FALSE |
| 654 | 1.85E−13 | 0.42371187 | 4.54E−09 | all | AL021396.1 | TRUE | FALSE |
| 655 | 2.17E−13 | 0.09878683 | 5.33E−09 | all | AC087241.2 | TRUE | FALSE |
| 656 | 3.02E−13 | 0.16792632 | 7.43E−09 | all | AL157756.1 | TRUE | FALSE |
| 657 | 3.07E−13 | 0.3850362 | 7.56E−09 | all | AC072061.1 | TRUE | FALSE |
| 658 | 3.33E−13 | 0.29909544 | 8.20E−09 | all | TULP2 | TRUE | FALSE |
| 659 | 3.89E−13 | −0.2870151 | 9.57E−09 | all | UHMK1 | TRUE | FALSE |
| 660 | 5.40E−13 | 0.85055803 | 1.33E−08 | all | PPP1R15A | TRUE | FALSE |
| 661 | 5.93E−13 | 0.20942868 | 1.46E−08 | all | AL133523.1 | TRUE | FALSE |
| 662 | 6.40E−13 | 0.32985939 | 1.58E−08 | all | SPART-AS1 | TRUE | FALSE |
| 663 | 6.41E−13 | 0.16538252 | 1.58E−08 | all | AL353135.1 | TRUE | FALSE |
| 664 | 7.68E−13 | 0.4543014 | 1.89E−08 | all | PIGA | TRUE | FALSE |
| 665 | 1.07E−12 | 0.33277828 | 2.63E−08 | all | YPEL5 | TRUE | FALSE |
| 666 | 1.13E−12 | −0.3288529 | 2.79E−08 | all | ZBTB37 | TRUE | FALSE |
| 667 | 1.29E−12 | 0.11251652 | 3.17E−08 | all | AC012485.3 | TRUE | FALSE |
| 668 | 1.30E−12 | −0.2295043 | 3.21E−08 | all | IGIP | TRUE | FALSE |
| 669 | 1.44E−12 | 0.20066491 | 3.54E−08 | all | AL139393.3 | TRUE | FALSE |
| 670 | 1.56E−12 | −0.4672125 | 3.83E−08 | all | ZNF780B | TRUE | FALSE |
| 671 | 1.65E−12 | 0.31843596 | 4.07E−08 | all | AC092431.1 | TRUE | FALSE |
| 672 | 1.67E−12 | 1.26344778 | 4.12E−08 | all | Z93241.1 | TRUE | TRUE |
| 673 | 1.89E−12 | 0.11226123 | 4.64E−08 | all | AL591846.2 | TRUE | FALSE |
| 674 | 1.99E−12 | −0.3312197 | 4.90E−08 | all | TMEM168 | TRUE | FALSE |
| 675 | 2.13E−12 | 0.19765022 | 5.23E−08 | all | AC008897.2 | TRUE | FALSE |
| 676 | 2.49E−12 | 0.22461795 | 6.12E−08 | all | AC005476.2 | TRUE | FALSE |
| 677 | 2.50E−12 | 0.29004426 | 6.14E−08 | all | AC005332.1 | TRUE | FALSE |
| 678 | 3.42E−12 | 0.54708909 | 8.41E−08 | all | AF213884.3 | TRUE | FALSE |
| 679 | 3.64E−12 | 0.13939769 | 8.96E−08 | all | SLC25A30-AS1 | TRUE | FALSE |
| 680 | 3.77E−12 | 0.20075584 | 9.29E−08 | all | AL353147.1 | TRUE | FALSE |
| 681 | 3.78E−12 | 0.16557889 | 9.31E−08 | all | AL022069.1 | TRUE | FALSE |
| 682 | 4.05E−12 | 0.31730767 | 9.98E−08 | all | CAMTA1-DT | TRUE | FALSE |
| 683 | 4.15E−12 | 0.17412256 | 1.02E−07 | all | PXT1 | TRUE | FALSE |
| 684 | 4.17E−12 | 0.5616091 | 1.03E−07 | all | GSG1 | TRUE | FALSE |
| 685 | 4.40E−12 | 0.25804722 | 1.08E−07 | all | AC098818.2 | TRUE | FALSE |
| 686 | 4.53E−12 | 0.21174795 | 1.12E−07 | all | C17orf64 | TRUE | FALSE |
| 687 | 5.10E−12 | 0.8207475 | 1.25E−07 | all | AC011444.3 | TRUE | FALSE |
| 688 | 5.12E−12 | 0.57277614 | 1.26E−07 | all | AC025171.3 | TRUE | FALSE |
| 689 | 5.69E−12 | 0.58812991 | 1.40E−07 | all | KLHL15 | TRUE | FALSE |
| 690 | 5.76E−12 | 0.34965197 | 1.42E−07 | all | AC144652.1 | TRUE | FALSE |
| 691 | 5.99E−12 | 0.3882092 | 1.47E−07 | all | AL121601.1 | TRUE | FALSE |
| 692 | 6.23E−12 | 0.57769021 | 1.53E−07 | all | AC072022.2 | TRUE | FALSE |
| 693 | 6.64E−12 | 0.82375794 | 1.63E−07 | all | OTUD1 | TRUE | FALSE |
| 694 | 7.04E−12 | −0.3338525 | 1.73E−07 | all | OIP5-AS1 | TRUE | FALSE |
| 695 | 8.64E−12 | 0.41977916 | 2.13E−07 | all | AC004854.2 | TRUE | FALSE |
| 696 | 8.88E−12 | 0.75860282 | 2.19E−07 | all | HECW2 | TRUE | FALSE |
| 697 | 1.03E−11 | 0.51376155 | 2.54E−07 | all | AL645728.1 | TRUE | FALSE |
| 698 | 1.07E−11 | 0.118923 | 2.63E−07 | all | AL022329.1 | TRUE | FALSE |
| 699 | 1.16E−11 | 0.51599006 | 2.87E−07 | all | FAM234B | TRUE | FALSE |
| 700 | 1.20E−11 | 1.48391066 | 2.95E−07 | all | JUN | TRUE | TRUE |
| 701 | 1.27E−11 | 0.09059005 | 3.13E−07 | all | AC026202.3 | TRUE | FALSE |
| 702 | 1.40E−11 | 0.50488602 | 3.44E−07 | all | AC020765.2 | TRUE | FALSE |
| 703 | 1.41E−11 | 0.17509105 | 3.46E−07 | all | AL035661.2 | TRUE | FALSE |
| 704 | 1.47E−11 | 0.19894131 | 3.63E−07 | all | C18orf65 | TRUE | FALSE |
| 705 | 1.51E−11 | 0.09520506 | 3.71E−07 | all | MAPK6-DT | TRUE | FALSE |
| 706 | 1.69E−11 | 0.2452515 | 4.17E−07 | all | AC009053.2 | TRUE | FALSE |
| 707 | 1.71E−11 | 0.24700042 | 4.22E−07 | all | C6orf52 | TRUE | FALSE |
| 708 | 1.82E−11 | −0.1842471 | 4.49E−07 | all | FP671120.7 | TRUE | FALSE |
| 709 | 2.08E−11 | 0.45817336 | 5.12E−07 | all | AL451085.1 | TRUE | FALSE |
| 710 | 2.14E−11 | 0.22589849 | 5.27E−07 | all | AL096677.1 | TRUE | FALSE |
| 711 | 2.27E−11 | 0.22591022 | 5.59E−07 | all | AC013400.1 | TRUE | FALSE |
| 712 | 2.42E−11 | 0.31437313 | 5.95E−07 | all | SCN11A | TRUE | FALSE |
| 713 | 2.54E−11 | 0.22164946 | 6.25E−07 | all | AL136038.3 | TRUE | FALSE |
| 714 | 2.85E−11 | 0.34212461 | 7.02E−07 | all | SMG7-AS1 | TRUE | FALSE |
| 715 | 3.29E−11 | 0.29984554 | 8.10E−07 | all | NANOS3 | TRUE | FALSE |
| 716 | 3.34E−11 | 0.56122943 | 8.22E−07 | all | KLF6 | TRUE | FALSE |
| 717 | 3.47E−11 | 0.28664298 | 8.55E−07 | all | AC005355.1 | TRUE | FALSE |
| 718 | 4.09E−11 | 0.20553985 | 1.01E−06 | all | CAGE1 | TRUE | FALSE |
| 719 | 4.12E−11 | 0.1969127 | 1.01E−06 | all | MIR17HG | TRUE | FALSE |
| 720 | 4.14E−11 | 0.3178696 | 1.02E−06 | all | LINC01465 | TRUE | FALSE |
| 721 | 5.34E−11 | 0.13364829 | 1.31E−06 | all | HIF1A-AS1 | TRUE | FALSE |
| 722 | 5.65E−11 | 0.30041127 | 1.39E−06 | all | PRRG2 | TRUE | FALSE |
| 723 | 6.21E−11 | 0.35384807 | 1.53E−06 | all | TM4SF20 | TRUE | FALSE |
| 724 | 6.24E−11 | 0.46093768 | 1.54E−06 | all | LINC02265 | TRUE | FALSE |
| 725 | 6.95E−11 | 0.13168462 | 1.71E−06 | all | AC073352.2 | TRUE | FALSE |
| 726 | 7.07E−11 | −0.3044712 | 1.74E−06 | all | LINC01355 | TRUE | FALSE |
| 727 | 7.69E−11 | −0.3110803 | 1.89E−06 | all | ZBTB41 | TRUE | FALSE |
| 728 | 7.88E−11 | 0.24934913 | 1.94E−06 | all | AC123595.1 | TRUE | FALSE |
| 729 | 8.31E−11 | 0.25336259 | 2.04E−06 | all | AC073934.1 | TRUE | FALSE |
| 730 | 8.43E−11 | −0.3304979 | 2.07E−06 | all | CLOCK | TRUE | FALSE |
| 731 | 8.54E−11 | 0.14277035 | 2.10E−06 | all | AC093677.2 | TRUE | FALSE |
| 732 | 9.32E−11 | 0.1109039 | 2.29E−06 | all | ZSWIM2 | TRUE | FALSE |
| 733 | 1.04E−10 | 1.17163713 | 2.56E−06 | all | AL691403.1 | TRUE | TRUE |
| 734 | 1.06E−10 | 0.24597746 | 2.60E−06 | all | ETFBKMT | TRUE | FALSE |
| 735 | 1.10E−10 | 0.41178429 | 2.70E−06 | all | AC012640.2 | TRUE | FALSE |
| 736 | 1.20E−10 | 0.28292774 | 2.95E−06 | all | CNGA4 | TRUE | FALSE |
| 737 | 1.22E−10 | 0.57503378 | 3.01E−06 | all | HIST1H3A | TRUE | FALSE |
| 738 | 1.25E−10 | −0.2953937 | 3.07E−06 | all | AL109628.2 | TRUE | FALSE |
| 739 | 1.36E−10 | 0.49543403 | 3.35E−06 | all | AC104984.2 | TRUE | FALSE |
| 740 | 1.45E−10 | 0.47584396 | 3.58E−06 | all | SREBF2-AS1 | TRUE | FALSE |
| 741 | 1.57E−10 | −0.3182937 | 3.87E−06 | all | ZNF175 | TRUE | FALSE |
| 742 | 1.59E−10 | 0.30132452 | 3.90E−06 | all | LINC02776 | TRUE | FALSE |
| 743 | 1.66E−10 | 0.36934317 | 4.08E−06 | all | AC087623.2 | TRUE | FALSE |
| 744 | 1.67E−10 | −0.431597 | 4.11E−06 | all | ZNF397 | TRUE | FALSE |
| 745 | 1.68E−10 | 0.1136725 | 4.14E−06 | all | AL022328.3 | TRUE | FALSE |
| 746 | 1.77E−10 | 0.47819401 | 4.37E−06 | all | AC023790.2 | TRUE | FALSE |
| 747 | 1.90E−10 | 0.2043077 | 4.68E−06 | all | AC124242.1 | TRUE | FALSE |
| 748 | 1.93E−10 | 0.1505041 | 4.74E−06 | all | AC127002.2 | TRUE | FALSE |
| 749 | 2.01E−10 | 0.25081613 | 4.95E−06 | all | LINC02539 | TRUE | FALSE |
| 750 | 2.28E−10 | 0.33149425 | 5.60E−06 | all | AC139099.2 | TRUE | FALSE |
| 751 | 2.43E−10 | 0.33745696 | 5.97E−06 | all | AC093635.1 | TRUE | FALSE |
| 752 | 2.61E−10 | 0.0957145 | 6.42E−06 | all | OSR2 | TRUE | FALSE |
| 753 | 2.66E−10 | 0.55709898 | 6.55E−06 | all | AL138720.1 | TRUE | FALSE |
| 754 | 2.73E−10 | 0.33321406 | 6.72E−06 | all | CASP9 | TRUE | FALSE |
| 755 | 2.75E−10 | 0.3321658 | 6.76E−06 | all | AC115618.1 | TRUE | FALSE |
| 756 | 2.81E−10 | 0.16631757 | 6.92E−06 | all | Z98742.4 | TRUE | FALSE |
| 757 | 2.99E−10 | 0.20706024 | 7.35E−06 | all | AC138304.1 | TRUE | FALSE |
| 758 | 3.00E−10 | 0.18000493 | 7.38E−06 | all | ENSA | TRUE | FALSE |
| 759 | 3.09E−10 | 0.34999694 | 7.61E−06 | all | SLC25A33 | TRUE | FALSE |
| 760 | 3.28E−10 | 0.18735419 | 8.07E−06 | all | AC022868.2 | TRUE | FALSE |
| 761 | 3.51E−10 | 0.19312255 | 8.64E−06 | all | AC012360.1 | TRUE | FALSE |
| 762 | 3.76E−10 | 0.26182374 | 9.25E−06 | all | AL031727.2 | TRUE | FALSE |
| 763 | 4.04E−10 | 0.36286711 | 9.94E−06 | all | AC010173.1 | TRUE | FALSE |
| 764 | 4.40E−10 | 0.49735104 | 1.08E−05 | all | ZFX-AS1 | TRUE | FALSE |
| 765 | 5.16E−10 | 0.27041476 | 1.27E−05 | all | AL355490.2 | TRUE | FALSE |
| 766 | 5.40E−10 | 0.12270395 | 1.33E−05 | all | AL360227.1 | TRUE | FALSE |
| 767 | 6.33E−10 | 0.89820636 | 1.56E−05 | all | MIR222HG | TRUE | FALSE |
| 768 | 6.58E−10 | 0.49253357 | 1.62E−05 | all | HIST1H2BC | TRUE | FALSE |
| 769 | 6.62E−10 | 0.12038958 | 1.63E−05 | all | UBE2L5 | TRUE | FALSE |
| 770 | 6.73E−10 | 0.24046355 | 1.66E−05 | all | LINC01010 | TRUE | FALSE |
| 771 | 6.86E−10 | 0.12242522 | 1.69E−05 | all | GLTPD2 | TRUE | FALSE |
| 772 | 7.14E−10 | −0.2980248 | 1.76E−05 | all | ANKRD30BL | TRUE | FALSE |
| 773 | 7.83E−10 | 0.3636825 | 1.93E−05 | all | IFRD1 | TRUE | FALSE |
| 774 | 9.38E−10 | 0.68530662 | 2.31E−05 | all | AC087239.1 | TRUE | FALSE |
| 775 | 1.06E−09 | 0.09648858 | 2.62E−05 | all | CT70 | TRUE | FALSE |
| 776 | 1.08E−09 | −0.1687791 | 2.65E−05 | all | AC093297.2 | TRUE | FALSE |
| 777 | 1.11E−09 | 0.29124786 | 2.74E−05 | all | AP001363.2 | TRUE | FALSE |
| 778 | 1.13E−09 | 0.87938001 | 2.78E−05 | all | AC044849.1 | TRUE | FALSE |
| 779 | 1.14E−09 | 0.35731397 | 2.80E−05 | all | AL137779.2 | TRUE | FALSE |
| 780 | 1.16E−09 | −0.2832258 | 2.84E−05 | all | TRAPPC6B | TRUE | FALSE |
| 781 | 1.22E−09 | 0.12309762 | 2.99E−05 | all | AL590096.1 | TRUE | FALSE |
| 782 | 1.23E−09 | −0.3195595 | 3.03E−05 | all | ZNF512 | TRUE | FALSE |
| 783 | 1.26E−09 | −0.3317262 | 3.09E−05 | all | NAPEPLD | TRUE | FALSE |
| 784 | 1.54E−09 | 0.09975472 | 3.80E−05 | all | AC132872.2 | TRUE | FALSE |
| 785 | 1.98E−09 | 0.73454839 | 4.87E−05 | all | AF111167.1 | TRUE | FALSE |
| 786 | 2.00E−09 | −0.3599462 | 4.91E−05 | all | NHLRC2 | TRUE | FALSE |
| 787 | 2.01E−09 | 0.16430734 | 4.95E−05 | all | RHCE | TRUE | FALSE |
| 788 | 2.12E−09 | 0.08942528 | 5.21E−05 | all | LINC02292 | TRUE | FALSE |
| 789 | 2.18E−09 | −0.3693591 | 5.37E−05 | all | AC007406.5 | TRUE | FALSE |
| 790 | 2.18E−09 | 0.43327725 | 5.37E−05 | all | THAP9 | TRUE | FALSE |
| 791 | 2.23E−09 | 0.24071875 | 5.48E−05 | all | YME1L1 | TRUE | FALSE |
| 792 | 2.40E−09 | 0.11393242 | 5.91E−05 | all | AP003680.1 | TRUE | FALSE |
| 793 | 2.41E−09 | −0.1231272 | 5.93E−05 | all | AC095032.1 | TRUE | FALSE |
| 794 | 2.41E−09 | 0.08494904 | 5.94E−05 | all | AC007881.3 | TRUE | FALSE |
| 795 | 2.52E−09 | 0.11634858 | 6.20E−05 | all | AC007686.4 | TRUE | FALSE |
| 796 | 2.81E−09 | 0.1222816 | 6.92E−05 | all | LINC01952 | TRUE | FALSE |
| 797 | 2.85E−09 | −0.263248 | 7.02E−05 | all | ZNF700 | TRUE | FALSE |
| 798 | 3.00E−09 | 0.15424561 | 7.37E−05 | all | AC006207.1 | TRUE | FALSE |
| 799 | 3.02E−09 | 0.21896099 | 7.42E−05 | all | TMEM202-AS1 | TRUE | FALSE |
| 800 | 3.04E−09 | 0.10762312 | 7.47E−05 | all | RUVBL1-AS1 | TRUE | FALSE |
| 801 | 3.04E−09 | 0.3002371 | 7.47E−05 | all | LINC01344 | TRUE | FALSE |
| 802 | 3.07E−09 | 0.31735359 | 7.56E−05 | all | HIST1H4A | TRUE | FALSE |
| 803 | 3.24E−09 | 0.32193003 | 7.96E−05 | all | AC105384.1 | TRUE | FALSE |
| 804 | 3.38E−09 | 0.33430684 | 8.31E−05 | all | LINC02541 | TRUE | FALSE |
| 805 | 3.59E−09 | −0.2890372 | 8.83E−05 | all | TMLHE-AS1 | TRUE | FALSE |
| 806 | 3.80E−09 | 0.67467578 | 9.34E−05 | all | DRAIC | TRUE | FALSE |
| 807 | 3.81E−09 | 1.13389548 | 9.38E−05 | all | AL450992.3 | TRUE | TRUE |
| 808 | 3.90E−09 | 0.0990365 | 9.60E−05 | all | IQCJ-SCHIP1 | TRUE | FALSE |
| 809 | 4.01E−09 | 0.31122346 | 9.86E−05 | all | LINC00309 | TRUE | FALSE |
| 810 | 4.09E−09 | 0.11266516 | 0.00010055 | all | TMEM52B | TRUE | FALSE |
| 811 | 4.41E−09 | 0.04707192 | 0.00010861 | all | IGFL2-AS1 | TRUE | FALSE |
| 812 | 4.48E−09 | 0.18208087 | 0.00011013 | all | AC002456.1 | TRUE | FALSE |
| 813 | 4.57E−09 | 0.17481471 | 0.00011242 | all | PMEL | TRUE | FALSE |
| 814 | 4.68E−09 | 0.31545822 | 0.00011516 | all | ADPGK-AS1 | TRUE | FALSE |
| 815 | 4.80E−09 | 0.10434554 | 0.00011823 | all | AC010240.3 | TRUE | FALSE |
| 816 | 5.29E−09 | −0.3745208 | 0.00013019 | all | ZNF234 | TRUE | FALSE |
| 817 | 5.33E−09 | 0.5945373 | 0.00013105 | all | CITED2 | TRUE | FALSE |
| 818 | 5.83E−09 | −0.3659226 | 0.00014352 | all | TRIM13 | TRUE | FALSE |
| 819 | 6.12E−09 | 1.02067285 | 0.00015058 | all | AC020916.1 | TRUE | TRUE |
| 820 | 6.73E−09 | 0.24687852 | 0.00016553 | all | AC079807.1 | TRUE | FALSE |
| 821 | 6.83E−09 | −0.1543623 | 0.00016816 | all | FP236383.4 | TRUE | FALSE |
| 822 | 7.06E−09 | 0.09058611 | 0.00017382 | all | ULBP1 | TRUE | FALSE |
| 823 | 7.09E−09 | 0.11348609 | 0.00017442 | all | GORAB-AS1 | TRUE | FALSE |
| 824 | 7.17E−09 | 0.29476941 | 0.00017653 | all | AL022069.3 | TRUE | FALSE |
| 825 | 7.36E−09 | 0.14009337 | 0.00018104 | all | AC092053.2 | TRUE | FALSE |
| 826 | 8.21E−09 | 0.12787424 | 0.00020205 | all | AC027307.3 | TRUE | FALSE |
| 827 | 8.32E−09 | −0.3402668 | 0.0002047 | all | ABHD18 | TRUE | FALSE |
| 828 | 8.86E−09 | 0.32400274 | 0.00021798 | all | LINC01970 | TRUE | FALSE |
| 829 | 9.27E−09 | 0.28510183 | 0.00022812 | all | EIF2AK3-DT | TRUE | FALSE |
| 830 | 9.97E−09 | 0.21422327 | 0.00024525 | all | AC093462.1 | TRUE | FALSE |
| 831 | 1.05E−08 | 0.09296428 | 0.00025853 | all | AL391839.2 | TRUE | FALSE |
| 832 | 1.06E−08 | 0.27455015 | 0.00026102 | all | SLC19A2 | TRUE | FALSE |
| 833 | 1.11E−08 | 0.52620228 | 0.00027229 | all | ZNF487 | TRUE | FALSE |
| 834 | 1.22E−08 | −0.3829926 | 0.00029901 | all | SEC22A | TRUE | FALSE |
| 835 | 1.24E−08 | −0.3355545 | 0.00030395 | all | ZNF12 | TRUE | FALSE |
| 836 | 1.25E−08 | 0.25498521 | 0.0003068 | all | NCBP2AS2 | TRUE | FALSE |
| 837 | 1.40E−08 | 0.12233932 | 0.00034544 | all | AL691403.2 | TRUE | FALSE |
| 838 | 1.46E−08 | 0.05573881 | 0.00036045 | all | AC012640.1 | TRUE | FALSE |
| 839 | 1.58E−08 | 0.16103057 | 0.0003883 | all | LINC01185 | TRUE | FALSE |
| 840 | 1.60E−08 | 0.31766457 | 0.00039394 | all | AC004917.1 | TRUE | FALSE |
| 841 | 1.63E−08 | 0.13185831 | 0.00040208 | all | AC024940.1 | TRUE | FALSE |
| 842 | 1.74E−08 | 0.13115031 | 0.00042757 | all | AL035411.3 | TRUE | FALSE |
| 843 | 1.92E−08 | 0.21660294 | 0.00047212 | all | HIPK1-AS1 | TRUE | FALSE |
| 844 | 1.95E−08 | 0.25057063 | 0.00047878 | all | AL161421.1 | TRUE | FALSE |
| 845 | 2.06E−08 | 0.14682515 | 0.00050756 | all | LINC02828 | TRUE | FALSE |
| 846 | 2.22E−08 | 0.10189558 | 0.00054712 | all | AP000845.1 | TRUE | FALSE |
| 847 | 2.34E−08 | 0.76272457 | 0.00057523 | all | AL512603.2 | TRUE | FALSE |
| 848 | 2.52E−08 | 0.18237287 | 0.00062083 | all | POPDC2 | TRUE | FALSE |
| 849 | 2.60E−08 | 0.11202623 | 0.00064048 | all | AC099541.1 | TRUE | FALSE |
| 850 | 2.74E−08 | 0.24066512 | 0.00067455 | all | AC093510.1 | TRUE | FALSE |
| 851 | 2.83E−08 | 0.0743275 | 0.00069598 | all | AC087482.1 | TRUE | FALSE |
| 852 | 3.01E−08 | 0.13959617 | 0.00074184 | all | AC004938.2 | TRUE | FALSE |
| 853 | 3.06E−08 | 0.47519325 | 0.0007537 | all | ITPRIP | TRUE | FALSE |
| 854 | 3.15E−08 | 0.62673191 | 0.00077559 | all | PTCH2 | TRUE | FALSE |
| 855 | 3.26E−08 | 0.08641284 | 0.00080309 | all | AC013270.1 | TRUE | FALSE |
| 856 | 3.27E−08 | 0.93798376 | 0.00080431 | all | AL021155.5 | TRUE | FALSE |
| 857 | 3.32E−08 | −0.236864 | 0.00081777 | all | RC3H2 | TRUE | FALSE |
| 858 | 3.33E−08 | 0.17855682 | 0.00082043 | all | AC084871.3 | TRUE | FALSE |
| 859 | 3.43E−08 | −0.2852495 | 0.00084335 | all | DCAF10 | TRUE | FALSE |
| 860 | 3.86E−08 | 0.09362049 | 0.0009497 | all | LINC00346 | TRUE | FALSE |
| 861 | 3.93E−08 | −0.3800371 | 0.00096795 | all | TMEM161B- | TRUE | FALSE |
| | | | | | AS1 | | |
| 862 | 3.96E−08 | 0.3099255 | 0.00097408 | all | AMZ1 | TRUE | FALSE |
| 863 | 4.27E−08 | −0.3274043 | 0.0010507 | all | AC005261.1 | TRUE | FALSE |
| 864 | 4.42E−08 | 1.29100249 | 0.0010864 | all | FOSB | TRUE | TRUE |
| 865 | 4.42E−08 | −0.321789 | 0.00108776 | all | NUP43 | TRUE | FALSE |
| 866 | 4.74E−08 | 0.11458721 | 0.00116722 | all | AC025031.2 | TRUE | FALSE |
| 867 | 4.82E−08 | −0.2984345 | 0.0011849 | all | ZNF33A | TRUE | FALSE |
| 868 | 4.83E−08 | 0.89819034 | 0.00118791 | all | KLF4 | TRUE | FALSE |
| 869 | 5.40E−08 | 0.14746442 | 0.00132929 | all | AC092718.1 | TRUE | FALSE |
| 870 | 5.46E−08 | 0.2968913 | 0.00134405 | all | NFKBIB | TRUE | FALSE |
| 871 | 5.57E−08 | 0.26457713 | 0.00137047 | all | AP001437.2 | TRUE | FALSE |
| 872 | 6.00E−08 | 0.22972186 | 0.00147549 | all | SLC1A2 | TRUE | FALSE |
| 873 | 6.08E−08 | −0.2539449 | 0.00149698 | all | PIGN | TRUE | FALSE |
| 874 | 6.23E−08 | 0.32202662 | 0.00153207 | all | PLK2 | TRUE | FALSE |
| 875 | 6.34E−08 | 0.14189695 | 0.0015588 | all | U91328.2 | TRUE | FALSE |
| 876 | 6.47E−08 | 0.22787233 | 0.00159241 | all | AC092343.1 | TRUE | FALSE |
| 877 | 7.04E−08 | −0.2670257 | 0.00173215 | all | JRK | TRUE | FALSE |
| 878 | 7.04E−08 | 0.23650198 | 0.00173343 | all | OSGIN2 | TRUE | FALSE |
| 879 | 7.05E−08 | 0.09019063 | 0.00173422 | all | AC009292.2 | TRUE | FALSE |
| 880 | 7.22E−08 | 0.23864938 | 0.00177663 | all | GPR137C | TRUE | FALSE |
| 881 | 7.41E−08 | −0.3066697 | 0.00182345 | all | UQCC1 | TRUE | FALSE |
| 882 | 7.80E−08 | 0.11204132 | 0.00191858 | all | GTF2IRD1 | TRUE | FALSE |
| 883 | 8.18E−08 | −0.2787774 | 0.00201297 | all | ZBED5 | TRUE | FALSE |
| 884 | 8.82E−08 | 0.05441812 | 0.00216909 | all | AC007785.1 | TRUE | FALSE |
| 885 | 9.70E−08 | −0.240586 | 0.0023857 | all | RNF170 | TRUE | FALSE |
| 886 | 9.70E−08 | 0.16099121 | 0.00238646 | all | LINC02357 | TRUE | FALSE |
| 887 | 9.70E−08 | 0.43026681 | 0.00238695 | all | C6orf99 | TRUE | FALSE |
| 888 | 9.80E−08 | 0.09956391 | 0.00241031 | all | Z99572.1 | TRUE | FALSE |
| 889 | 1.01E−07 | 0.18152222 | 0.00249118 | all | AC139019.1 | TRUE | FALSE |
| 890 | 1.16E−07 | −0.3028649 | 0.00285358 | all | MFAP3 | TRUE | FALSE |
| 891 | 1.24E−07 | 0.11544677 | 0.00305433 | all | AC245033.2 | TRUE | FALSE |
| 892 | 1.28E−07 | 0.0747788 | 0.00314775 | all | CEP83-DT | TRUE | FALSE |
| 893 | 1.30E−07 | 0.38180483 | 0.00319959 | all | LINC02728 | TRUE | FALSE |
| 894 | 1.33E−07 | 1.66377451 | 0.00326414 | all | CXCL8 | TRUE | TRUE |
| 895 | 1.37E−07 | −0.2509249 | 0.00336227 | all | MIGA1 | TRUE | FALSE |
| 896 | 1.38E−07 | 0.16272926 | 0.00338655 | all | AC092053.3 | TRUE | FALSE |
| 897 | 1.41E−07 | 0.08548066 | 0.00347253 | all | LINC00677 | TRUE | FALSE |
| 898 | 1.42E−07 | 0.09530881 | 0.00349369 | all | ZAR1L | TRUE | FALSE |
| 899 | 1.43E−07 | 0.11630088 | 0.00351173 | all | AC091114.1 | TRUE | FALSE |
| 900 | 1.46E−07 | 0.25107272 | 0.00359746 | all | PTS | TRUE | FALSE |
| 901 | 1.50E−07 | 0.81258047 | 0.00368161 | all | ATF3 | TRUE | FALSE |
| 902 | 1.51E−07 | 0.1028228 | 0.00371356 | all | AL353708.1 | TRUE | FALSE |
| 903 | 1.52E−07 | 0.07247116 | 0.00373125 | all | AC016526.4 | TRUE | FALSE |
| 904 | 1.52E−07 | 0.07480528 | 0.00374778 | all | CATSPERZ | TRUE | FALSE |
| 905 | 1.55E−07 | 0.13623052 | 0.00381712 | all | AC008115.3 | TRUE | FALSE |
| 906 | 1.59E−07 | −0.2775253 | 0.00390057 | all | DBT | TRUE | FALSE |
| 907 | 1.60E−07 | 0.06025929 | 0.00394263 | all | ANKRD40CL | TRUE | FALSE |
| 908 | 1.62E−07 | −0.3274161 | 0.00397463 | all | MFSD4B | TRUE | FALSE |
| 909 | 1.64E−07 | 0.38132199 | 0.00404106 | all | AP000943.2 | TRUE | FALSE |
| 910 | 1.66E−07 | 0.25446753 | 0.00409317 | all | AL163973.2 | TRUE | FALSE |
| 911 | 1.68E−07 | 0.16632147 | 0.00413249 | all | AL356234.2 | TRUE | FALSE |
| 912 | 1.70E−07 | 0.12742964 | 0.00419045 | all | ZNF695 | TRUE | FALSE |
| 913 | 1.75E−07 | −0.2674555 | 0.00429735 | all | KRIT1 | TRUE | FALSE |
| 914 | 1.76E−07 | 0.33675866 | 0.00433949 | all | CH25H | TRUE | FALSE |
| 915 | 1.81E−07 | −0.40902 | 0.00444616 | all | HCG17 | TRUE | FALSE |
| 916 | 1.88E−07 | 0.08554715 | 0.0046172 | all | CT69 | TRUE | FALSE |
| 917 | 1.89E−07 | 0.37772066 | 0.00464397 | all | TEX41 | TRUE | FALSE |
| 918 | 1.93E−07 | −0.1964106 | 0.0047394 | all | AC024060.2 | TRUE | FALSE |
| 919 | 2.01E−07 | −0.2399771 | 0.00495543 | all | LRIG2 | TRUE | FALSE |
| 920 | 2.06E−07 | 0.16663616 | 0.00507231 | all | RB1-DT | TRUE | FALSE |
| 921 | 2.08E−07 | 0.12402214 | 0.00511973 | all | ADGRV1 | TRUE | FALSE |
| 922 | 2.16E−07 | 0.36274932 | 0.00531813 | all | CUBN | TRUE | FALSE |
| 923 | 2.21E−07 | 0.0726026 | 0.00543214 | all | LINC02457 | TRUE | FALSE |
| 924 | 2.23E−07 | 0.28124324 | 0.00547819 | all | AP001269.4 | TRUE | FALSE |
| 925 | 2.25E−07 | 0.18996722 | 0.00552956 | all | AC012629.2 | TRUE | FALSE |
| 926 | 2.36E−07 | 0.71918247 | 0.00580498 | all | NFKBIA | TRUE | FALSE |
| 927 | 2.39E−07 | 0.06466737 | 0.00586867 | all | C16orf71 | TRUE | FALSE |
| 928 | 2.40E−07 | 0.14954395 | 0.00589745 | all | CFAP45 | TRUE | FALSE |
| 929 | 2.48E−07 | 0.38696528 | 0.00610173 | all | CBX4 | TRUE | FALSE |
| 930 | 2.50E−07 | −0.2856217 | 0.00616191 | all | PHC3 | TRUE | FALSE |
| 931 | 2.64E−07 | 0.18743619 | 0.00650072 | all | AC009630.1 | TRUE | FALSE |
| 932 | 2.67E−07 | 0.64984687 | 0.00657009 | all | JUNB | TRUE | FALSE |
| 933 | 2.76E−07 | 0.22799842 | 0.00680021 | all | HIST1H2AD | TRUE | FALSE |
| 934 | 2.77E−07 | 0.06690386 | 0.0068243 | all | AC107398.5 | TRUE | FALSE |
| 935 | 2.77E−07 | 0.20392468 | 0.00682758 | all | AP000919.3 | TRUE | FALSE |
| 936 | 2.96E−07 | 0.11236776 | 0.00728449 | all | AL356776.2 | TRUE | FALSE |
| 937 | 2.98E−07 | −0.4328615 | 0.00732966 | all | AC007216.4 | TRUE | FALSE |
| 938 | 3.05E−07 | 0.15089853 | 0.0075084 | all | AC103724.3 | TRUE | FALSE |
| 939 | 3.05E−07 | 0.08215777 | 0.00750908 | all | AL606760.3 | TRUE | FALSE |
| 940 | 3.15E−07 | −0.310272 | 0.00775724 | all | TTC13 | TRUE | FALSE |
| 941 | 3.20E−07 | 0.20166291 | 0.00787899 | all | AL162377.1 | TRUE | FALSE |
| 942 | 3.23E−07 | 0.28500912 | 0.00793735 | all | AC023157.3 | TRUE | FALSE |
| 943 | 3.27E−07 | 0.43634915 | 0.00804017 | all | GZF1 | TRUE | FALSE |
| 944 | 3.36E−07 | −0.3330167 | 0.00827029 | all | MBNL3 | TRUE | FALSE |
| 945 | 3.47E−07 | 0.21470303 | 0.0085276 | all | SDR42E2 | TRUE | FALSE |
| 946 | 3.57E−07 | 0.1326268 | 0.00877946 | all | LINC01554 | TRUE | FALSE |
| 947 | 3.57E−07 | 0.35017505 | 0.00878288 | all | SLC38A2 | TRUE | FALSE |
| 948 | 3.80E−07 | 0.93726504 | 0.0093456 | all | CD83 | TRUE | FALSE |
| 949 | 4.03E−07 | −0.2470499 | 0.00992625 | all | CRLF3 | TRUE | FALSE |
| 950 | 4.09E−07 | 0.07400282 | 0.010052 | all | AL590133.1 | TRUE | FALSE |
| 951 | 4.14E−07 | 0.71847224 | 0.01018663 | all | AC103591.3 | TRUE | FALSE |
| 952 | 4.52E−07 | 0.13026345 | 0.01111184 | all | CFAP43 | TRUE | FALSE |
| 953 | 4.54E−07 | 0.08865638 | 0.01116414 | all | MBOAT4 | TRUE | FALSE |
| 954 | 4.55E−07 | 0.31937602 | 0.01118353 | all | AL390957.1 | TRUE | FALSE |
| 955 | 4.56E−07 | 0.06953925 | 0.01122384 | all | AC100835.1 | TRUE | FALSE |
| 956 | 4.69E−07 | 0.44088108 | 0.01153704 | all | CDHR2 | TRUE | FALSE |
| 957 | 4.86E−07 | 0.80023204 | 0.01197014 | all | AC239799.2 | TRUE | FALSE |
| 958 | 4.88E−07 | 0.14979302 | 0.01201505 | all | SDCBP2 | TRUE | FALSE |
| 959 | 5.19E−07 | −0.3479146 | 0.01277621 | all | TRIM56 | TRUE | FALSE |
| 960 | 5.20E−07 | 0.09872992 | 0.01279222 | all | AL512288.1 | TRUE | FALSE |
| 961 | 5.29E−07 | 0.17997935 | 0.01302425 | all | ARRDC3-AS1 | TRUE | FALSE |
| 962 | 5.42E−07 | 0.11203948 | 0.01332609 | all | AC083843.2 | TRUE | FALSE |
| 963 | 5.50E−07 | −0.1979898 | 0.0135249 | all | EXOC5 | TRUE | FALSE |
| 964 | 5.53E−07 | 0.14739133 | 0.01361538 | all | AP005329.1 | TRUE | FALSE |
| 965 | 5.63E−07 | 0.20753526 | 0.01385403 | all | AL627422.2 | TRUE | FALSE |
| 966 | 5.66E−07 | 0.08521633 | 0.01392343 | all | AP000640.2 | TRUE | FALSE |
| 967 | 5.76E−07 | 0.17855162 | 0.01418504 | all | AC025181.2 | TRUE | FALSE |
| 968 | 6.26E−07 | 0.5442403 | 0.01541448 | all | CSRNP1 | TRUE | FALSE |
| 969 | 6.38E−07 | 0.49317713 | 0.01568978 | all | ZEB2-AS1 | TRUE | FALSE |
| 970 | 6.54E−07 | 0.27956686 | 0.0160859 | all | ERCC1 | TRUE | FALSE |
| 971 | 6.58E−07 | −0.2822955 | 0.01618854 | all | CMTR2 | TRUE | FALSE |
| 972 | 6.63E−07 | −0.1094633 | 0.01630335 | all | AC006480.2 | TRUE | FALSE |
| 973 | 6.75E−07 | 0.15524498 | 0.01660792 | all | AC096751.2 | TRUE | FALSE |
| 974 | 6.77E−07 | 0.11159558 | 0.01665506 | all | AC092117.1 | TRUE | FALSE |
| 975 | 6.85E−07 | 0.11965354 | 0.01684773 | all | AP2M1 | TRUE | FALSE |
| 976 | 6.85E−07 | −0.3416861 | 0.01685078 | all | CBR4 | TRUE | FALSE |
| 977 | 6.87E−07 | −0.2884395 | 0.0169056 | all | ZNF251 | TRUE | FALSE |
| 978 | 6.97E−07 | 0.25923868 | 0.01715383 | all | TOB1-AS1 | TRUE | FALSE |
| 979 | 7.02E−07 | −0.3652253 | 0.01727027 | all | ZNF235 | TRUE | FALSE |
| 980 | 7.20E−07 | 0.07273394 | 0.01772168 | all | AC026461.3 | TRUE | FALSE |
| 981 | 7.32E−07 | 0.087085 | 0.01801966 | all | Z83847.1 | TRUE | FALSE |
| 982 | 7.50E−07 | −0.3902622 | 0.01845413 | all | CEPT1 | TRUE | FALSE |
| 983 | 7.51E−07 | 0.10980538 | 0.01848844 | all | SF3B2 | TRUE | FALSE |
| 984 | 7.64E−07 | 0.1391608 | 0.01880913 | all | RPP38-DT | TRUE | FALSE |
| 985 | 8.14E−07 | 0.35396865 | 0.02003503 | all | MZF1-AS1 | TRUE | FALSE |
| 986 | 8.29E−07 | 0.06362614 | 0.02040464 | all | LINC02579 | TRUE | FALSE |
| 987 | 8.48E−07 | −0.2948604 | 0.02086868 | all | ZNF594 | TRUE | FALSE |
| 988 | 9.03E−07 | 0.24115019 | 0.02222716 | all | AC096577.1 | TRUE | FALSE |
| 989 | 9.48E−07 | 0.14061489 | 0.02332241 | all | FRMD6-AS1 | TRUE | FALSE |
| 990 | 9.75E−07 | 0.24960027 | 0.02398055 | all | RNF139 | TRUE | FALSE |
| 991 | 9.87E−07 | −0.2310507 | 0.02429536 | all | AL513320.1 | TRUE | FALSE |
| 992 | 1.03E−06 | 0.06628524 | 0.02532972 | all | AC109454.3 | TRUE | FALSE |
| 993 | 1.05E−06 | 0.0750037 | 0.02583668 | all | MIR378D2HG | TRUE | FALSE |
| 994 | 1.06E−06 | 0.37465686 | 0.02604235 | all | KIF9 | TRUE | FALSE |
| 995 | 1.07E−06 | 0.27613397 | 0.02624328 | all | AL392046.1 | TRUE | FALSE |
| 996 | 1.08E−06 | −0.2843571 | 0.02661126 | all | ZNF81 | TRUE | FALSE |
| 997 | 1.09E−06 | 0.09064726 | 0.02685285 | all | AL117344.2 | TRUE | FALSE |
| 998 | 1.10E−06 | 0.07724272 | 0.02699144 | all | AC100812.1 | TRUE | FALSE |
| 999 | 1.16E−06 | 0.07539734 | 0.02856059 | all | AC026333.3 | TRUE | FALSE |
| 1000 | 1.17E−06 | 0.32476519 | 0.02881872 | all | AC092164.1 | TRUE | FALSE |
| 1001 | 1.18E−06 | 0.40129904 | 0.0289501 | all | VIM-AS1 | TRUE | FALSE |
| 1002 | 1.22E−06 | 0.09525758 | 0.03008373 | all | AL121761.1 | TRUE | FALSE |
| 1003 | 1.23E−06 | 0.37833768 | 0.03031247 | all | IFFO2 | TRUE | FALSE |
| 1004 | 1.31E−06 | 0.22655413 | 0.03231337 | all | AL513303.1 | TRUE | FALSE |
| 1005 | 1.35E−06 | 0.29900235 | 0.03310288 | all | MEPCE | TRUE | FALSE |
| 1006 | 1.38E−06 | 0.15842875 | 0.03406495 | all | USP12-AS2 | TRUE | FALSE |
| 1007 | 1.40E−06 | −0.2771311 | 0.03434369 | all | PIP5K1A | TRUE | FALSE |
| 1008 | 1.40E−06 | 0.08850224 | 0.03434375 | all | SKIDA1 | TRUE | FALSE |
| 1009 | 1.41E−06 | 0.08306436 | 0.03460443 | all | AC125611.3 | TRUE | FALSE |
| 1010 | 1.41E−06 | 0.95597867 | 0.03465459 | all | LINC00910 | TRUE | FALSE |
| 1011 | 1.46E−06 | −0.3047147 | 0.0358164 | all | LRRC69 | TRUE | FALSE |
| 1012 | 1.46E−06 | 0.08284865 | 0.03591272 | all | CYP51A1-AS1 | TRUE | FALSE |
| 1013 | 1.54E−06 | −0.2589443 | 0.03798187 | all | FBXL4 | TRUE | FALSE |
| 1014 | 1.59E−06 | 0.04887311 | 0.03918948 | all | AP001922.5 | TRUE | FALSE |
| 1015 | 1.60E−06 | 0.16951248 | 0.03930709 | all | AC073195.1 | TRUE | FALSE |
| 1016 | 1.61E−06 | 0.12305143 | 0.03965158 | all | PRMT5-AS1 | TRUE | FALSE |
| 1017 | 1.61E−06 | 0.45792382 | 0.0397259 | all | ZBTB10 | TRUE | FALSE |
| 1018 | 1.65E−06 | 0.21753742 | 0.04048722 | all | GCC2-AS1 | TRUE | FALSE |
| 1019 | 1.66E−06 | 0.38704161 | 0.04087367 | all | KMT2E-AS1 | TRUE | FALSE |
| 1020 | 1.66E−06 | −0.2474929 | 0.04093876 | all | AL365356.1 | TRUE | FALSE |
| 1021 | 1.67E−06 | 0.14743408 | 0.04112571 | all | LINC00271 | TRUE | FALSE |
| 1022 | 1.69E−06 | 0.10133186 | 0.04165865 | all | AC108471.2 | TRUE | FALSE |
| 1023 | 1.70E−06 | 0.19523497 | 0.04185086 | all | YTHDF3-AS1 | TRUE | FALSE |
| 1024 | 1.72E−06 | 0.44007682 | 0.04222256 | all | WDR74 | TRUE | FALSE |
| 1025 | 1.76E−06 | 0.08129512 | 0.04321928 | all | AC117394.2 | TRUE | FALSE |
| 1026 | 1.77E−06 | 0.03867368 | 0.04350337 | all | AL356608.3 | TRUE | FALSE |
| 1027 | 1.77E−06 | −0.2640726 | 0.04359764 | all | PAPOLG | TRUE | FALSE |
| 1028 | 1.77E−06 | 0.24448756 | 0.04367433 | all | SPAG6 | TRUE | FALSE |
| 1029 | 1.79E−06 | 0.31109604 | 0.04415373 | all | SIK1B | TRUE | FALSE |
| 1030 | 1.80E−06 | 0.19604026 | 0.04420645 | all | LINC01412 | TRUE | FALSE |
| 1031 | 1.81E−06 | 0.11911773 | 0.04449499 | all | AL121990.1 | TRUE | FALSE |
| 1032 | 1.81E−06 | 0.10600969 | 0.04450794 | all | SOD2-OT1 | TRUE | FALSE |
| 1033 | 1.83E−06 | 0.33405673 | 0.04491885 | all | DDX59-AS1 | TRUE | FALSE |
| 1034 | 1.83E−06 | −0.3528419 | 0.04502393 | all | AC009061.2 | TRUE | FALSE |
| 1035 | 1.83E−06 | 0.21016739 | 0.04511422 | all | CCNK | TRUE | FALSE |
| 1036 | 1.86E−06 | −0.2427656 | 0.04569398 | all | RETREG3 | TRUE | FALSE |
| 1037 | 1.90E−06 | 0.05780343 | 0.0468124 | all | SCAANT1 | TRUE | FALSE |
| 1038 | 1.92E−06 | 0.11425659 | 0.04718338 | all | LINC00167 | TRUE | FALSE |
| 1039 | 1.99E−06 | 0.06518324 | 0.04890028 | all | AC080013.5 | TRUE | FALSE |
| 1040 | 2.02E−06 | −0.2388836 | 0.04971787 | all | LNPEP | TRUE | FALSE |
| 1041 | 2.02E−06 | −0.250567 | 0.04976833 | all | UMAD1 | TRUE | FALSE |

TABLE 5
Analysis of differential gene expression as assessed by ADT

| | | | | | | | significant + |
|---|---|---|---|---|---|---|---|
| | | avg_log2FC | | | | | fold change |
| | | positive = enriched | | | | significant | (p_val_{—} |
| | | in Ficoll, | | | | (p_val_{—} | adjusted < 0.05 |
| | | negative = enriched | p_val_{—} | | | adjusted < | and abs(avg_{—} |
| | p_val | in Cryo-PRO | adjusted | cell type | Protein | 0.05?) | log2FC) > 1?) |
| 1 | 1.35E−05 | −0.7426974 | 0.00184285 | Monocyte | adt-B3GAT1 | TRUE | FALSE |
| 2 | 8.91E−05 | −0.2278418 | 0.0122076 | Monocyte | adt-ITGAM | TRUE | FALSE |
| 3 | 2.76E−05 | −0.7031895 | 0.00377628 | B. cell | adt-B3GAT1 | TRUE | FALSE |
| 4 | 0.00016047 | −0.8976596 | 0.02198428 | B. cell | adt-SELL | TRUE | FALSE |
| 5 | 0.0001172 | −0.1615757 | 0.01605599 | T. cell | adt-C0224 | TRUE | FALSE |
| 6 | 0.00026982 | −0.6175448 | 0.03696482 | DC | adt-TFRC | TRUE | FALSE |

### Example 5: Cryo-PRO and Ficoll Yield Similar Cell Type and Substate Abundances

Defining the composition of circulating immune cells and their active substates on a per-patient level is an informative application of scRNA-seq. In sepsis, there is substantial heterogeneity in the distribution of immune cell types and states between patients that is thought to be a major contributor to differences in illness trajectory, outcomes, and response to therapies. To evaluate the congruence between methods for characterizing immune cell profiles in sepsis, fractional abundances for each cell type and substate for samples processed using both Ficoll and Cryo-PRO methods was computed. Fractional abundance of cell type was defined as the number of cells of a particular type (e.g., B cells) divided by the number of all PBMCs combined. For substates, fractional abundance was defined as the number of cells assigned to a substate divided by the total number of cells of that cell type (e.g., number of CD16+ monocytes/total number of monocytes). Fractional abundances were compared between paired Ficoll and Cryo-PRO samples from each of the 24 subjects by investigating their correlation (FIG. 4A, FIG. 4B, and FIG. 4C). Substate abundances were calculated for B cells, T cells, and monocytes. Dendritic cells were present at very low abundance and resolved into only two substates, while natural killer cells did not demonstrate unique substates; therefore substate abundances were not computed for these cell types.

Proportions of cell types were significantly correlated between methods (FIG. 4A), with Pearson correlations (R) ranging between 0.88 and 0.93 (p<0.001 for all comparisons). There were significant positive correlations between methods for fractional abundance of most substates (R values from 0.76 to 0.96; p<0.001); the exceptions were memory B cells (R=0.42, NS, not significant) and gamma delta T cells (R=0.28, NS) (FIG. 4B). Within monocytes, correlations across methods were higher for CD16+ monocytes (R=0.96, p<0.001) than for CD14+ MS1 (R=0.78, p<0.001) and classical CD14+ (R=0.83, p<0.001). The poorly correlated memory B cell proportions may be influenced by the fact that naive and memory B cells exist on a continuum such that stochastic differences in clustering may have a bigger impact in assignment between these similar substates (FIG. 4C). Correlations for the CD14+ monocyte substates may have been affected by transcriptional similarity as well (FIG. 4D). Gamma delta T cells were present in very low numbers across each method, with enhanced effects of outliers likely impacting correlations. Patient-level cell substate proportions showed a generally high level of similarity between methods (FIG. 4E and FIG. 4F).

To evaluate the robustness of the Cryo-PRO approach, the technical reproducibility of scRNA-seq results for the same blood samples processed at different clinical sites was assessed. Cell type and substate abundances was compared for the 8 patients whose samples were processed at both MGH and BIDMC. Proportions of major cell types (monocytes, B cells, and T cells) were highly correlated when the patient sample was simultaneously processed at different clinical sites for Ficoll (R values from 0.83 to 0.96, p<0.001) (FIG. 4C, left panel) and Cryo-PRO (R values from 0.86 to 0.99, p<0.001) (FIG. 4C, right panel). Dendritic cells (Ficoll R=0.05, Cryo-PRO R=0.44; both NS) and natural killer cells (Ficoll R=0.34, Cryo-PRO R=0.72; both NS) were poorly correlated between sites, possibly due to small overall cell counts and variable yield between processing runs that exaggerate differences in cell proportions. For cell substates, correlations were significant for nearly all substates of monocytes, T cells, B cells, and dendritic cells for each method between sites (FIG. 4D), though for some substates including MS1, cross-site correlations were slightly lower for Cryo-PRO (FIG. 4D, right column) than Ficoll (FIG. 4D, left column).

FIG. 4G shows a scatter plot of dendritic cell substate proportion from Ficoll and Cryo-PRO. Each point represents the proportion of one cell substate from one patient sample, as measured by each method. Each cell substate is represented by a different color and trendline. Proportion is the number of cells of one cell substate divided by the total number of dendritic cells from that patient sample. Patient-paired Ficoll: Cryo-PRO samples are plotted to assess correlation in method for each patient. Pearson's correlations (R) are shown for all correlations (*p<0.05, **p<0.01, ***p<0.001).

### Example 6: Single-Cell TCR Sequencing Yields Comparable Repertoire Capture from Ficoll and Cryo-PRO Samples

T cell lymphopenia and altered T cell receptor (TCR) diversity are recognized features of sepsis and its recovery. Previous studies have demonstrated that patients with septic shock exhibit reduced TCR repertoire breadth early after onset. Persistent contraction of the TCR repertoire has been associated with increased mortality, higher rates of nosocomial infection, and reactivation of latent viral infections such as cytomegalovirus. These findings underscore the clinical importance of tracking TCR repertoire dynamics in sepsis. Capturing this data alongside paired single-cell gene expression data provides valuable information on immune dysfunction within cellular substates, as well as gene expression programs associated with changes in clonotype diversity.

To determine whether paired single-cell transcriptomic and TCR profiling can be preserved using Cryo-PRO, the 10× Genomics 5′v2 Immune Profiling workflow (Methods) was applied to matched patient samples processed using either Ficoll or Cryo-PRO. This strategy allows for joint recovery of full-length V(D)J sequences and gene expression data from the same cells. The yield and quality of TCR sequencing was compared across both methods.

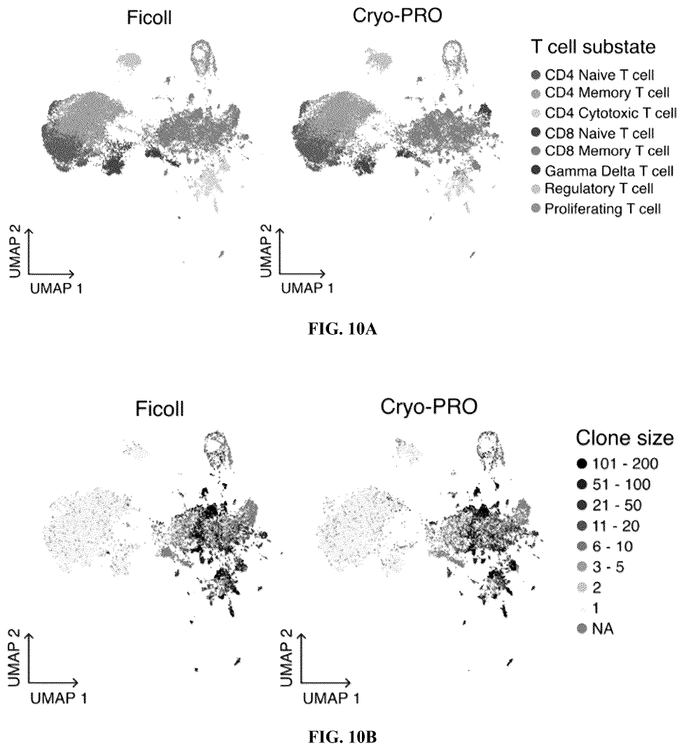

Among Ficoll-processed samples, TCR sequences were recovered from 21,876 cells, representing 98.6% of T cells with transcriptomic data. For Cryo-PRO samples, TCR sequences were recovered from 18,447 cells (96.5% of T cells with transcriptomic data). Overall, expanded and unique clonotypes were represented similarly on UMAP projections of Ficoll and Cryo-PRO T cells (FIG. 10A and FIG. 10B). As expected, effector and cytotoxic cells (CD4+ cytotoxic T cells and CD8+ memory T cells), which expand during acute immune responses to infection, displayed the highest levels of clonal expansion by both methods, whereas naïve CD4+ and CD8+ T cell substates displayed greater sequence diversity and fewer expanded clonotypes (FIG. 10A and FIG. 10B).

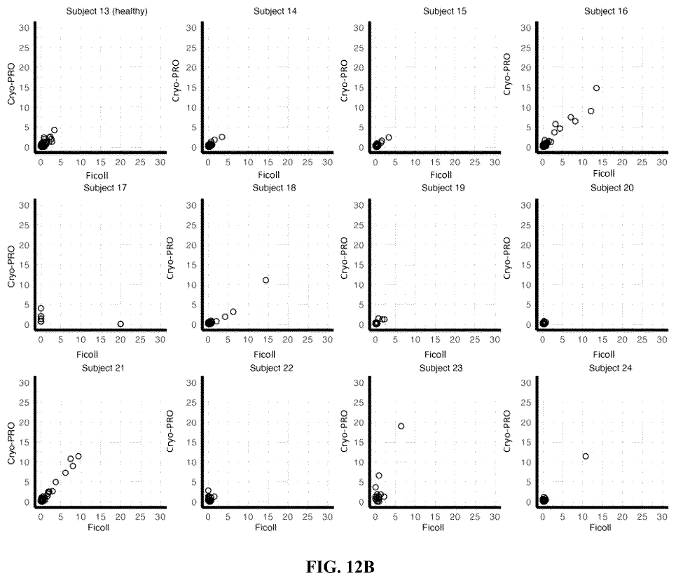

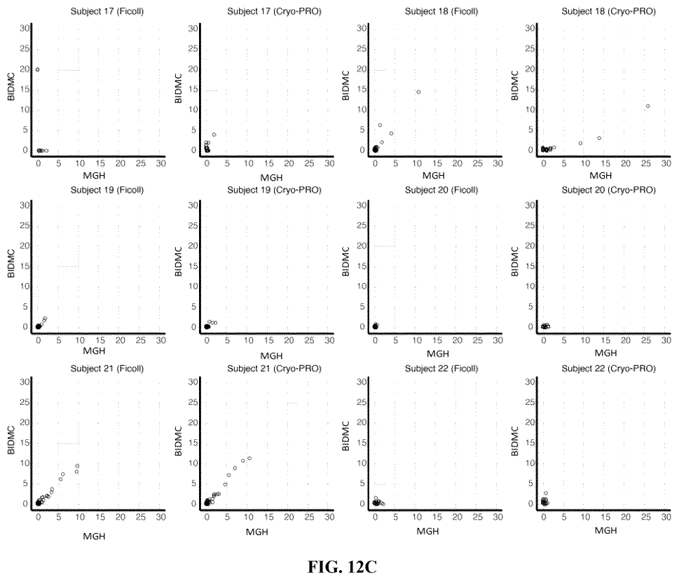

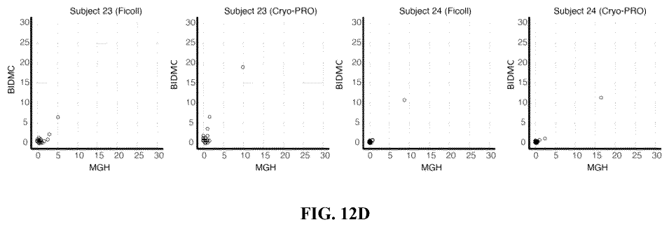

The exact nucleotide sequence of the captured TCR clonotypes was then compared between methods. While many clonotypes appear at very low frequencies (i.e., detected only once) in a given sample, expanded clonotypes from the same patient should be detected at higher frequencies in both Ficoll and Cryo-PRO samples. The abundance of each matching unique TCR sequence from the same patient as a proportion of the total TCR sequences captured for that sample was calculated and compared between methods and processing centers. Patient-matched Ficoll and Cryo-PRO TCR sequence proportions were substantially similar (Pearson's R=0.47, p<0.001) (FIGS. 12A-B). Significant overlap was also found between processing centers for the eight patients processed at MGH and BIDMC for Ficoll samples (R=0.38, p<0.001 between processing centers) and Cryo-PRO samples (R=0.79, p<0.001 between processing centers) (FIGS. 12C-D).

FIG. 4H and FIG. 4I are graphs showing clonal expansion proportions for samples processed at single centers and technical duplicate samples processed at both centers. Samples from the same patient processed using different methods are shown next to each other. In FIG. 4I, the corresponding pair of technical duplicates processed at the non-origin site are shown subsequently, with the labeled site indicating where each sample was processed. PRO denotes Cryo-PRO. Per-patient clonotypes were generally similar between methods, both in the proportion and the exact sequence of expanded clones (see e.g., FIG. 4H and FIG. 4I; FIG. 12A-12B). Discrepancies in clonal proportions were generally attributable to low T cell recovery from at least one of the samples (FIG. 4H and FIG. 4I). For patients with samples processed at both EDs, similar trends were observed, with TCR clonal proportions and sequences closely resembling each other between processing center and method for each patient profiled (FIG. 4H and FIG. 4I; FIG. 12C-12D).

FIG. 12A-12D show a series of graphs providing a comparative analysis of identical TCR receptor clones detected by Cryo-PRO versus Ficoll methods from individual patients. FIG. 12A and FIG. 12B directly compare between Cryo-PRO (y-axis) and Ficoll (x-axis), whereas FIG. 12C and FIG. 12D compare across recruitment sites (BIDMC, y-axis; vs MGH, x-axis) for one method or the other (Ficoll or Cryo-PRO, indicated in each panel's title).

### Example 7: Ficoll and Cryo-PRO Methods Preserve Cellular Functions Required for Phagocytosis

Functional activity was next measured in the cryopreserved cells. One key function for monocytes is phagocytosis, which requires cells to detect the presence of a pathogen, encapsulate it inside a phagosome, and initiate microbial killing and degradation via fusion with lysosomes and subsequent exposure to hydrolytic enzymes and acidic conditions. Detection of phagocytosis therefore requires coordinated cellular signaling pathways, cytoskeletal rearrangement, and functional organelles. Phagocytic activity was measured in PBMCs from samples processed using Ficoll and Cryo-PRO as a means of assessing cell viability, function, and responsiveness to environmental stimuli.

Ficoll and Cryo-PRO samples (one of each from nine sepsis patients and one healthy subject) were collected, preserved, and frozen as described herein. Ficoll and Cryo-PRO samples underwent all of the previously described processing steps for sequencing before the flow cytometry sorting step, including the red blood cell depletion step for Cryo-PRO samples only. Cell suspensions were then incubated with E. coli pHrodo Bioparticles (Invitrogen), which fluoresce only in the acidic conditions of a phagolysosome. After incubation, cells were fluorescently stained for viability, CD45, CD15, and CD14, and analyzed with flow cytometry.

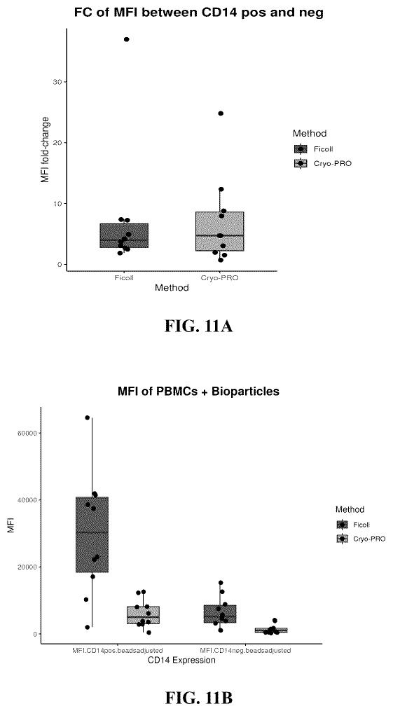

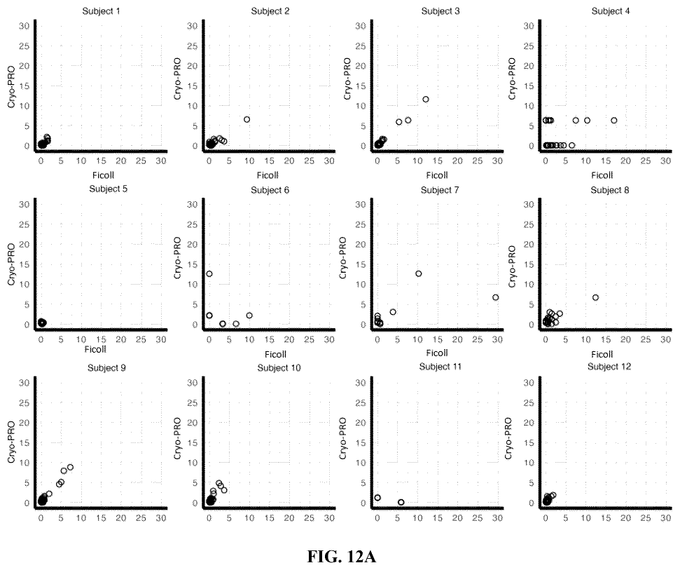

Phagocytic activity was measured as the mean fluorescence intensity (MFI) of the pHrodo dye within live CD45+ CD15− cells, stratified by CD14 expression. CD14+ monocytes are the most abundant phagocytes within PBMCs and were expected to show higher MFI compared to the CD14− fraction, which consists primarily of lymphocytes with low phagocytic activity, but does include CD16+ monocytes and dendritic cells. Across all patients, clear differences were observed in phagocytic signal between CD14+ PBMCs and CD14− PBMCs, despite substantial inter-individual variability. On average, CD14+ cells exhibited a ˜4.5 fold (Ficoll), and ˜3.5 fold (Cryo-PRO) higher MFI than CD14-cells in the presence of the bioparticles (FIG. 11A). The MFI of CD14+ cells from Ficoll was generally higher than CD14+ cells from the corresponding Cryo-PRO sample (FIG. 11B). For Ficoll, the MFI fold-change had a mean of 4.68 and a median of 3.98 with a standard deviation of 10.54. For Ficoll, more specifically, the MFI mean for CD14+ was 29,869.9 RFU, with a median of 30,239.5 RFU; and the MFI mean for CD14− was 6,378.3 RFU, with a median of 5133 RFU. For Cryo-PRO, the MFI fold-change had a mean of 4.09 and a median of 4.72 with a standard deviation of 7.23. For Cryo-PRO, more specifically, the MFI mean for CD14+ was 6064.1 RFU with a median of 4946 RFU; and the MFI mean for CD14− was 1482.3 RFU with a median of 975 RFU. This is possibly due to greater levels of contaminating cells in Cryo-PRO samples such as granulocytes that compete with CD14+ monocytes for bioparticle uptake. Although comparisons of phagocytic activity in CD14+ PBMCs between methods could not be standardized for this reason, the clear increase in MFI in CD14+ cells compared to CD14− cells within Ficoll and Cryo-PRO methods demonstrates that preserved CD14+ PBMCs retained detectable levels of their hallmark functional activity in both methods.

All patents and publications mentioned in the specification are indicative of the levels of skill of those skilled in the art to which the disclosure pertains. All references cited in this disclosure are incorporated by reference to the same extent as if each reference had been incorporated by reference in its entirety individually.

One skilled in the art would readily appreciate that the present disclosure is well adapted to carry out the objects and obtain the ends and advantages mentioned, as well as those inherent therein. The methods and compositions described herein as presently representative of preferred embodiments are exemplary and are not intended as limitations on the scope of the disclosure. Changes therein and other uses will occur to those skilled in the art, which are encompassed within the spirit of the disclosure, are defined by the scope of the claims.

In addition, where features or aspects of the disclosure are described in terms of Markush groups or other grouping of alternatives, those skilled in the art will recognize that the disclosure is also thereby described in terms of any individual member or subgroup of members of the Markush group or other group.

All methods described herein can be performed in any suitable order unless otherwise indicated herein or otherwise clearly contradicted by context. The use of any and all examples, or exemplary language (e.g., “such as”) provided herein, is intended merely to better illuminate the disclosure and does not pose a limitation on the scope of the disclosure unless otherwise claimed. No language in the specification should be construed as indicating any non-claimed element as essential to the practice of the disclosure.

It will be readily apparent to one skilled in the art that varying substitutions and modifications can be made to the present disclosure herein without departing from the scope and spirit of the present disclosure. Thus, such additional embodiments are within the scope of the present disclosure and the following claims. The present disclosure teaches one skilled in the art to test various combinations and/or substitutions of chemical modifications described herein toward generating conjugates possessing improved contrast, diagnostic and/or imaging activity. Therefore, the specific embodiments described herein are not limiting and one skilled in the art can readily appreciate that specific combinations of the modifications described herein can be tested without undue experimentation toward identifying conjugates possessing improved contrast, diagnostic and/or imaging activity.

The inventors expect skilled artisans to employ such variations as appropriate, and the inventors intend for the disclosure to be practiced otherwise than as specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context. Those skilled in the art will recognize, or be able to ascertain using no more than routine experimentation, many equivalents to the specific embodiments of the disclosure described herein. Such equivalents are intended to be encompassed by the following claims.

We claim:

1. A method of cryopreserving a blood sample and isolating peripheral blood mononuclear cells (PBMCs) from the blood sample, comprising:
a) obtaining the blood sample;
b) mixing the blood sample with dimethyl sulfoxide (DMSO) to create a blood sample-DMSO mixture that does not comprise a serum supplement;
c) freezing the blood sample-DMSO mixture within four hours of obtaining the blood sample;
d) thawing the blood sample-DMSO mixture;
e) mixing the thawed blood sample-DMSO mixture with a buffer to create a buffered blood sample-DMSO mixture, wherein the buffer comprises phosphate buffered saline (PBS), ethylenediaminetetraacetic acid (EDTA), and a serum supplement;
f) depleting red blood cells from the buffered blood sample-DMSO mixture using a negative selection; and
g) performing flow cytometry on the depleted and buffered blood sample-DMSO mixture to isolate PBMCs.

2. The method of claim 1, wherein the blood sample-DMSO mixture comprises between about 5% and about 15% DMSO v/v.

3. The method of claim 1, wherein the serum supplement is fetal bovine serum (FBS), newborn calf serum (NCS), horse serum, human serum, platelet lysate, bovine serum albumin (BSA), serum replacement, tryptose phosphate broth (TPB), insulin-transferrin-selenium (ITS), KnockOut™ Serum Replacement (KSR), CryoStor, or any combination thereof.

4. The method of claim 1, wherein the method is performed without a centrifugation step.

5. The method of claim 1, wherein the depleting step comprises immunomagnetic depletion.

6. The method of claim 1, wherein the EDTA molarity is between about 1 mM and about 5 mM.

7. The method of claim 1, wherein freezing comprises decreasing the temperature of the blood sample-DMSO mixture by at least about 1 degree per minute; or wherein thawing comprises incubating the blood sample-DMSO mixture at 37° C. for about 1 minute 15 seconds.

8. The method of claim 1, wherein the blood sample is from a human subject.

9. The method of claim 1, further comprising:
h) assaying the isolated PBMCs using single-cell RNA sequencing (scRNA-seq).

10. The method of claim 9, wherein the scRNA-seq is droplet based scRNA-seq.

11. The method of claim 9, wherein the scRNA-seq is on more than one blood sample.

12. The method of claim 11, wherein the more than one blood sample is from at least two different subjects.

13. The method of claim 11, wherein the more than one blood sample is from the same subject.

14. The method of claim 9, further comprising generating an RNA library from the scRNA-seq.

15. A method of selecting a treatment for sepsis in a subject in need thereof, the method comprising:
identifying a sepsis-specific disease endotype in the subject comprising:
a) obtaining a blood sample;
b) incubating the blood sample from the subject with an aprotic solvent, to create a blood sample-aprotic solvent mixture that does not comprise serum;
c) freezing the blood sample-aprotic solvent mixture within four hours of obtaining the blood sample;
d) thawing the blood sample-aprotic solvent mixture;
e) mixing the thawed blood sample-aprotic solvent mixture with a buffer to create a buffered blood sample-aprotic solvent mixture, wherein the buffer comprises phosphate buffered saline (PBS), ethylenediaminetetraacetic acid (EDTA), and a serum supplement;
f) depleting red blood cells from the buffered blood sample-aprotic solvent mixture using a negative selection;
g) performing flow cytometry on the depleted and buffered blood sample-aprotic solvent mixture to isolate PBMCs;
h) assaying the isolated PBMCs using single-cell RNA sequencing;
i) analyzing the scRNA-seq data, thereby identifying a sepsis-specific disease endotype; and
selecting a treatment for sepsis in the subject based on the sepsis-specific disease endotype identified.

16. The method of claim 15, wherein the sepsis-specific disease endotype is selected from the group consisting of: Molecular Diagnosis and Risk Stratification of Sepsis (MARS) 1, MARS 2, MARS 3, MARS 4, Sepsis Response Signature (SRS) 1, SRS 2, Neutrophilic-Suppressive (NPS), Inflammatory (INF), Innate Host Defence (IHD), Interferon (IFN), and Adaptive (ADA); or the sepsis disease endotype is associated with neutrophil activation and immune suppression; associated with an increased pro-inflammatory response, associated with an increased NF-κB expression; associated with interleukin signaling; associated with increased IFN-α,β,γ; or associated with a variety of pathways including increased adaptive immunity.

17. A kit for cryopreserving and processing whole blood for single-cell RNA sequencing, the kit comprising:
a) dimethyl sulfoxide (DMSO);
b) a buffer comprising phosphate buffered saline (PBS), ethylenediaminetetraacetic acid (EDTA), and a serum supplement;
c) a red blood cell depletion reagent; and
d) instructions for use.

18. The kit of claim 17, wherein the red blood cell depletion reagent comprises immunomagnetic beads.

19. The kit of claim 17, further comprising flow cytometry reagents for isolating peripheral blood mononuclear cells (PBMCs).

20. The kit of claim 17, further comprising single cell RNA sequencing reagents.
